(19) United States
(12) Patent Application Publication
(10) Pub. No.: US 2021/0147497 A1
(43) Pub. Date: May 20, 2021

(54) COMPOSITIONS AND METHODS FOR TREATING OR PREVENTING CATECHOLAMINERGIC POLYMORPHIC VENTRICULAR TACHYCARDIA

(71) Applicant: CHILDREN'S MEDICAL CENTER CORPORATION, BOSTON, MA (US)

(72) Inventors: WILLIAM PU, BOSTON, MA (US); VASSILIOS BEZZERIDES, BOSTON, MA (US); DONGHUI ZHANG, BOSTON, MA (US)

(73) Assignee: CHILDREN'S MEDICAL CENTER CORPORATION, BOSTON, MA (US)

(21) Appl. No.: 16/628,162

(22) PCT Filed: Jul. 6, 2018
(86) PCT No.: PCT/US18/41043

§ 371 Date: Jan. 2, 2020

Related U.S. Application Data
(60) Provisional application No. 62/529,256, filed on Jul. 6, 2017.

(51) Int. Cl. C07K 14/47; G01N 33/50; C12N 15/86; C12N 7/00; A61P 9/06

(52) U.S. Cl. CPC C07K 14/4703; G01N 33/5061; C12N 15/86; C12N 7/00; A61K 38/00; C12N 2750/14143; C12N 2750/14171; G01N 2500/10; A61P 9/06

(57) ABSTRACT

The present invention features AIP peptide and polynucleotide compositions, methods of using such compositions for the treatment of CPVT, as well as a human induced pluripotent stem cell derived cardiomyocyte model, useful in characterizing agents that modulate myocardial conduction and contraction.

### CROSS REFERENCE TO RELATED APPLICATION

This application is an International Application which designated the U.S., and which claims the benefit under 35 U.S.C. § 119(e) of U.S. Provisional Application No. 62/529,256 filed on Jul. 6, 2017, the contents of which are incorporated herein by reference in its entirety.

### STATEMENT OF RIGHTS TO INVENTIONS MADE UNDER FEDERALLY SPONSORED RESEARCH

This invention was made with government support under Grant Nos: NIH U01 HL100401 and UG3 TR002145 awarded by the National Institutes of Health. The government has certain rights in the invention.

### BACKGROUND OF THE INVENTION

Catecholaminergic polymorphic ventricular tachycardia (CPVT) is a condition characterized by an abnormal heart rhythm, which affects as many as one in ten thousand people. Symptoms of CPVT include dizziness or fainting associated with exercise or emotional stress. Episodes of ventricular tachycardia may cause the heart to stop beating effectively (cardiac arrest), leading to sudden death in children and young adults without recognized heart abnormalities. Treatments for CPVT, include exercise restriction, the use of beta blockers, and automatic implantable cardioverter defibrillators. Other treatments are surgical sympathectomy and treatment with flecainide. Unfortunately, these treatments are not effective for all patients and are limited by patient compliance, medication side effects, or the risk of adverse events such as fatal electrical storms caused by implantable defibrillators.

### SUMMARY OF THE INVENTION

Embodiments of the disclosure herein are based, in part, to the discovery that the inhibition of CaMKII activation and subsequent downstream signaling significantly reduces the catecholamine-stimulated latent arrhythmia that is associated with mutations in the calcium ryanodine channel, RYR2. In in vivo experiments, the inventors showed that the peptide inhibitor, AIP, when expressed in vivo in cardiac tissues of CPVT model mice, inhibited arrhythmia in the mice. See Example 2, FIGS. 17 and 18. The inventors also found that the CaMKII-mediated phosphorylation of the serine residue at S2814 in RYR2 is essential for catecholamine-stimulated latent arrhythmic in CPVT mutations. Mutation of the serine to alanine reverses the aberrant Ca²⁺ spark frequency recorded for cardiac cells having CPVT-associated mutations in the RYR2 protein.

Accordingly, as described below, the present invention features Ca²⁺-calmodulin dependent kinase II (CaMKII) inhibitory peptides including autocamtide-2-related inhibitory peptide (AIP) and related peptides, and CaM-KNtide and related polypeptides (such as CN19o), and related polynucleotide compositions, and methods of using such compositions for the treatment of CPVT. The invention further provides CPVT induced pluripotent stem cell cardiomyocytes (iPSC-CMs) and methods of using them to characterize agents for the treatment of CPVT.

In one aspect, this disclosure provides a pharmaceutical composition comprising an effective amount of a vector encoding a CaMKII peptide inhibitor.

In one aspect, provided herein is a pharmaceutical composition comprising an effective amount of a vector encoding a CaMKII peptide inhibitor for use in the treatment of cardiac arrhythmia, for example, such as catecholaminergic polymorphic ventricular tachycardia (CPVT).

In one aspect, provided herein is a pharmaceutical composition comprising an effective amount of a vector encoding a CaMKII peptide inhibitor for use in the manufacture of medicament for the treatment of cardiac arrhythmia, for example, such as CPVT.

In another aspect, provided herein is an expression vector comprising a polynucleotide encoding a CaMKII peptide inhibitor.

In one aspect, provided herein is an expression vector comprising a polynucleotide encoding a CaMKII peptide inhibitor for use in the treatment of cardiac arrhythmia, for example, such as CPVT.

In one aspect, provided herein is an expression vector comprising a polynucleotide encoding a CaMKII peptide inhibitor for use in the manufacture of medicament for the treatment of cardiac arrhythmia, for example, such as CPVT.

In another aspect, provided herein is a cell comprising an expression vector comprising a polynucleotide encoding a CaMKII peptide inhibitor.

In one aspect, provided herein is a cell comprising an expression vector comprising a polynucleotide encoding a CaMKII peptide inhibitor for use in the treatment of cardiac arrhythmia, for example, such as CPVT.

In one aspect, provided herein is a cell comprising an expression vector comprising a polynucleotide encoding a CaMKII peptide inhibitor for use in the manufacture of medicament for the treatment of cardiac arrhythmia, for example, such as CPVT.

In another aspect, provided herein is a method for modulating a cardiac arrhythmia in a subject, the method comprising contacting a cell comprising a cardiac ryanodine channel (RYR2) with a CaMKII inhibitor, CaMKII peptide inhibitor or polynucleotide encoding the CaMKII peptide inhibitor.

In another aspect, provided herein is a method for inhibiting the phosphorylation of a ryanodine channel (RYR2) polypeptide in a subject, the method comprising contacting a cell comprising a cardiac ryanodine channel (RYR2) with a CAMKII inhibitor, CaMKII peptide inhibitor or polynucleotide encoding a CaMKII peptide inhibitor.

In another aspect, provided herein is a method of treating a subject comprising a mutation associated with a cardiac arrhythmia, the method comprising administering to the subject a CaMKII inhibitor, CaMKII peptide inhibitor, analog, or fragment thereof or polynucleotide encoding a CaMKII peptide inhibitor.

In another aspect, provided herein is a method of characterizing a cardiomyocyte, the method comprising monitoring cardiac conduction or contraction, or monitoring cardiac arrhythmia using an induced pluripotent stem cell derived cardiomyocyte expressing a cardiac ryanodine channel (RYR2) comprising a mutation associated with CPVT.

In another aspect, provided herein is a method of compound screening, the method comprising contacting an induced pluripotent stem cell derived cardiomyocyte expressing a cardiac ryanodine channel (RYR2) comprising a mutation associated with CPVT with a candidate agent and measuring cardiac conduction or contraction in the cell.

In one embodiment of any one aspect described, the CaMKII peptide inhibitor is AIP, CN19, CN19o, CN27, CN21, or an analog or fragment thereof.

In one embodiment of any one aspect described or any one of the prior embodiments, the CaMKII peptide inhibitor is operably linked to a promoter suitable for driving expression of the peptide in a mammalian cardiac cell.

In one embodiment of any one aspect described or any one of the prior embodiments, the vector is a pharmaceutical composition comprising an effective amount of an CaMKII peptide inhibitor, analog, or fragment thereof.

In one embodiment of any one aspect described or any one of the prior embodiments, the vector is a retroviral, adenoviral, or adeno-associated viral vector.

In one embodiment of any one aspect described or any one of the prior embodiments, the mutation is in a cardiac ryanodine channel (RYR2).

In one embodiment of any one aspect described or any one of the prior embodiments, the mutation is selected from the group consisting of RYR2R⁴⁶⁵¹¹, RYR2^{R1760}, RYR2^{D385N}, RYR2^{5404R}, and RYR2^{G3946S}.

In one embodiment of any one aspect described or any one of the prior embodiments, the method inhibits a cardiac arrhythmia.

In one embodiment of any one aspect described or any one of the prior embodiments, the method inhibits catecholaminergic polymorphic ventricular tachycardia in the subject.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Autocamtide-2-related inhibitory peptide (AIP)” is meant a peptide or fragment thereof comprising at least about 9-13 amino acids of KKALRRQEAVDAL (SEQ. ID. NO: 1) and having cardiac regulatory activity and/or CAMKII inhibitory activity. In one embodiment, the AIP peptide comprises one or more alterations in the peptide sequence. In one embodiment, the AIP peptide consists essentially of SEQ. ID. NO: 1. In another embodiment, the AIP peptide consists of SEQ. ID. NO: 1 or consists of about 9-13 contiguous amino acids of SEQ. ID. NO: 1. In one embodiment, the AIP peptide consists essentially of SEQ. ID. NO: 1 or consists essentially of about 9-13 contiguous amino acids of SEQ. ID. NO: 1. In other embodiment, the AIP peptide comprises one or more modified amino acids.

By “CAMKII inhibitor” is meant a peptide or small molecule that inhibits the activity of CAMKII. Exemplary inhibitors are known in the art (e.g., AIP, CN19, CN27, CN19o, CN21) and described, for example, by Coultrap et al., PLOS One e25245, Vol 6, Issue 10, 2011 and Pellicena et al., Frontiers in Pharmacology 21:1-20, 2014. Other inhibitors include, the following:

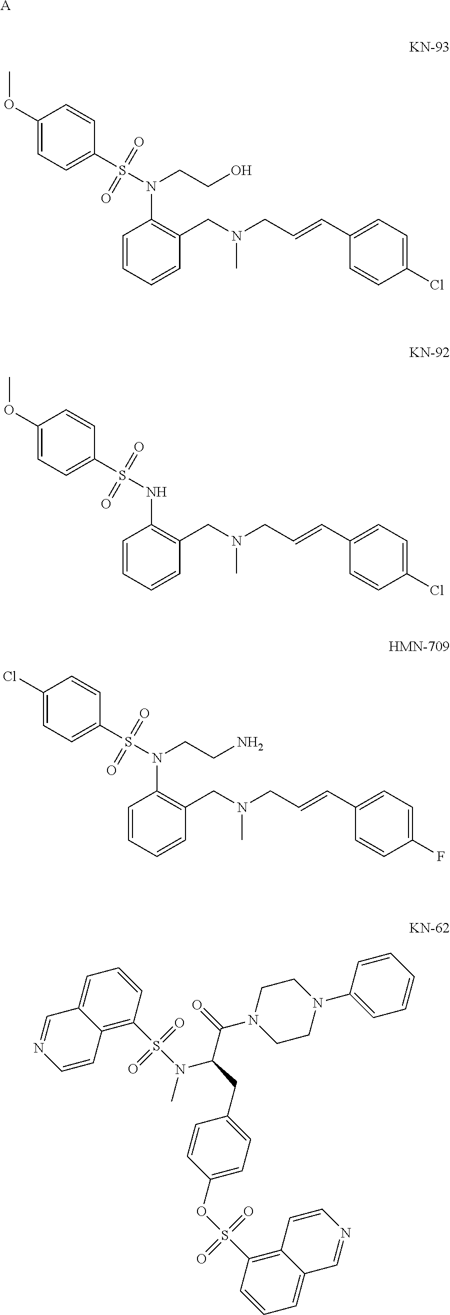

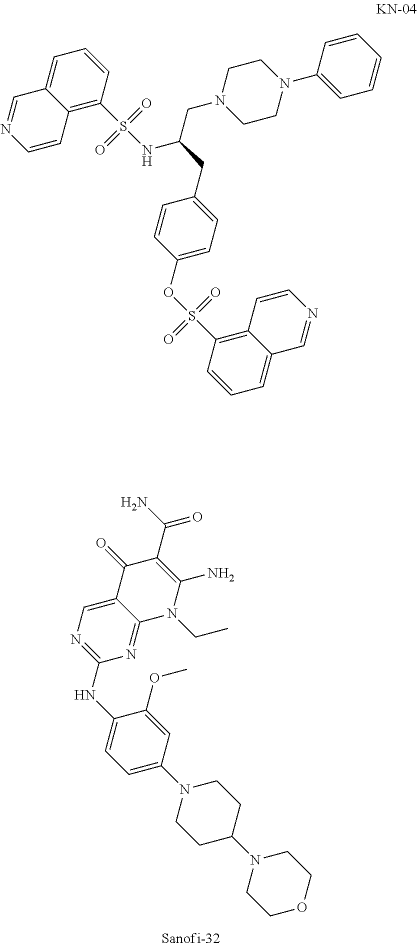 [SMILES: O=S(=O)(N[C@H](Cc1ccc(OS(=O)(=O)c2cccc3cnccc23)cc1)CN1CCN(c2ccccc2)CC1)c1cccc2cnccc12 ; CCn1c(N)c(C(N)=O)c(=O)c2cnc(Nc3ccc(N4CCC(N5CCOCC5)CC4)cc3OC)nc21]

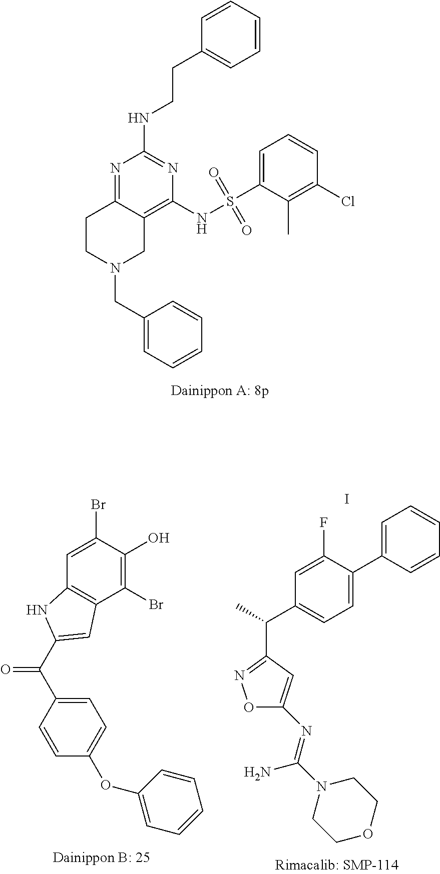

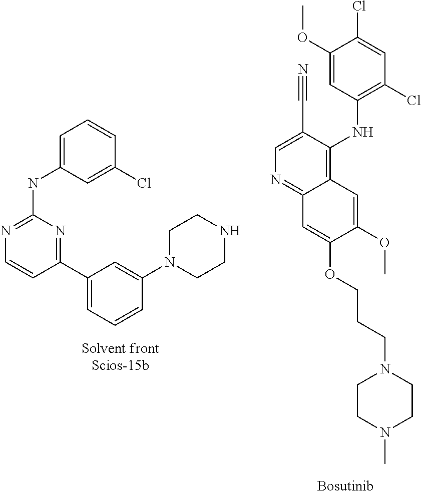 [SMILES: Clc1cccc(Nc2nccc(-c3cccc(N4CCNCC4)c3)n2)c1 ; COc1cc(Nc2c(C#N)cnc3cc(OCCCN4CCN(C)CC4)c(OC)cc23)c(Cl)cc1Cl]

By “AIP polynucleotide” is meant a polynucleotide that encodes an AIP peptide.

By “agent” is meant a peptide, polypeptide, nucleic acid molecule, or small compound.

By “ameliorate” is meant decrease, suppress, attenuate, diminish, arrest, or stabilize the development or progression of a disease.

By “alteration” in an AIP peptide means a change in the amino acid sequence of the AIP peptide.

For example, a polynucleotide analog retains the biological activity of a corresponding naturally-occurring polypeptide, while having certain biochemical modifications that enhance the analog's function relative to a naturally occurring polynucleotide. Such biochemical modifications could increase the analog's nuclease resistance, membrane permeability, or half-life, without altering, for example, functional activity, such as its protein encoding function. An analog may include a modified nucleic acid molecule.

The term “cardiomyocyte” as used herein broadly refers to a muscle cell of the heart. In one embodiment, a mammalian cardiac cell is a cardiomyocyte. In another embodiment, a cardiomyocyte that is differentiated from an induced pluripotent stem cell is a cardiomyocyte.

As used herein, the phrase “cardiovascular condition, disease or disorder” is intended to include all disorders characterized by insufficient, undesired or abnormal cardiac function, e.g. ischemic heart disease, cardiac arrhythmia, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury, genetic mutations, and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

The term “effective amount” as used herein refers to the amount of therapeutic agent of pharmaceutical composition, e.g., to express sufficient amount of the protein to reduce at least one or more symptom(s) of the disease or disorder, and relates to a sufficient amount of pharmacological composition to provide the desired effect. The phrase “therapeutically effective amount” as used herein, e.g., of an AIP peptide as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to, for example, effect a therapeutically or prophylactically significant reduction in a symptom or clinical marker associated with a cardiac dysfunction or disorder when administered to a typical subject who has a cardiovascular condition, disease or disorder.

With reference to the treatment of, for example, a cardiovascular condition or disease in a subject, the term “therapeutically effective amount” refers to the amount that is safe and sufficient to prevent or delay the development or a cardiovascular disease or disorder (e.g., cardiac arrhythmia). The amount can thus cure or cause the arrhythmia to be suppressed, or to cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285;H1797; 2003) and animal models acute myocardial infarction. (Yang Z, Am J Physiol Heart Circ. Physiol 282:H949: 2002; Guo Y, J Mol Cell Cardiol 33;825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals.

Subjects amenable to treatment by the methods as disclosed herein can be identified by any method to diagnose cardiac arrhythmia. Methods of diagnosing these conditions are well known by persons of ordinary skill in the art. By way of non-limiting example, cardiac arrhythmia can be diagnosed by electrocardiogram (ECG or EKG) which is a graphic recordation of cardiac activity, either on paper or a computer monitor.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “reduces” in the context cardiac arrhythmia described herein or in the context of symptoms is meant a reduction of at least 1%, at least 5%, at least 10%, at least 25%, at least 50%, at least 75%, or at least 100% of incidences of arrhythmia or symptoms, or severity of symptoms, including whole integer percentages from 1% to 100%.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween. In one embodiment, a reference AIP peptide is KKALRRQEAVDAL (SEQ. ID. NO: 1).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, N.Y., 2001); Berger and Kimmel (Guide to Molecular Cloning Techniques, 1987, Academic Press, N.Y.); and Sambrook et al., Molecular Cloning: A Laboratory Manual, Cold Spring Harbor Laboratory Press, N.Y..

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “modulate” refers to regulate or adjust to a certain degree.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

A “subject,” as used herein, includes any animal that exhibits a symptom of a monogenic disease, disorder, or condition that can be treated with the gene therapy vectors, cell-based therapeutics, and methods disclosed elsewhere herein. In preferred embodiments, a subject includes any animal that exhibits symptoms of a disease, disorder, or condition that can be treated with the gene therapy vectors, cell-based therapeutics, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (lower or higher amounts than a “normal” or “healthy” subject) of one or more physiological activities that can be modulated by therapy. A subject is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “tissue” refers to a group or layer of similarly specialized cells which together perform certain special functions. The term “tissue-specific” refers to a source or defining characteristic of cells from a specific tissue.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C show the characterization of Ca²⁺ oscillations in isolated iPSC-CM islands. FIG. 1A shows immunofluorescent images of WT, CPVTp, and CPVTe iPSC-CMs, stained for the sarcomeric marker ACTN2. The cell lines had indistinguishable appearance. Bar, 50 μm. FIG. 1B shows that Ca²⁺ sparks recorded from Fluo-4-loaded iPSC-CMs by confocal line scan imaging. Violin plot shows distribution of Ca²⁺ spark frequency. Number by each shape denotes number of cell clusters. FIG. 1C shows the Ca²⁺ oscillations recorded by confocal line scan imaging of isolated iPSC-CM islands. Arrows and arrowheads indicate aberrant early and delayed Ca²⁺ release events, respectively. Number by each shape denotes number of cell clusters. Steel Dwass non-parametric test with multiple testing correction; #, vs WT; †, vs WT+ISO; § vs CPVTp; ¥ vs CPVT. *, P<0.05; **, P<0.01; ***, P<0.001.

FIGS. 2A-2F show the opto-MTF engineered heart tissue for arrhythmia modeling. FIG. 2A shows the schematic of opto-MTF system to optically pace and optically measure tissue-level Ca²⁺ wave propagation and contraction. Cardiomyocyte programmed to express ChR2 are seeded on micro-molded gelatin with flexible cantilevers on one end. Focal illumination using optical fibers excites cells, resulting in Ca²⁺ wave propagation along the MTF and into the cantilevers. Ca²⁺ wave propagation is measured by fluorescent imaging of the Ca²⁺-sensitive dye X-Rhod-1, and mechanical contraction by darkfield imaging of the cantilevers. FIG. 2B shows confocal images of ACTN2-stained opto-MTF. Micro-molded gelatin induces iPSC-CMs to grow with their long axis aligned with the long axis of the MTF. FIG. 2C shows the excitation-contraction coupling in CPVTp opto-MTFs. Representative time lapse images show Ca²⁺ wave propagation and mechanical systole recorded induced by optogenetic point stimulation. FIG. 2D shows the Ca²⁺ traces that were recorded at the points labeled a-d in the right-most image of FIG. 2C. Vertical parallel lines across each trace indicate the optical pacing at the stimulation point. Activation time is the time to the maximal Ca²⁺ signal upstroke velocity. CaTD80 is the duration of the Ca²⁺ transient at 80% decay. FIG. 2E shows the spatial maps of activation time, Ca²⁺ wave speed and direction, and CaTD80 for WT and CPVTp opto-MTFs at 1.5 Hz pacing, demonstrating well-ordered behavior of both tissues. Bar, 1 mm. FIG. 2F shows the comparison of the frequency of after-depolarizations in spontaneously beating cell islands or opto-MTF tissue. Fisher's exact test: ***, P<0.001.

FIGS. 3A-3I show the characterization of CPVT opto-MTFs. FIG. 3A shows the time lapse images of CPVTp opto-MTF Ca²⁺ wavefront propagation and cantilever contraction. Ca²⁺ wavefronts, calculated from the temporal derivative of Ca²⁺ signals, show spiral wave re-entry. Bar, 1 mm. FIG. 3B shows the Ca²⁺ signal and contractile stress traces during re-entry. Representative example of CPVTp opto-MTF paced at 3 Hz. Vertical parallel lines across each trace indicate optical pacing at the stimulation site. FIG. 3C shows the occurrence of re-entry in CPVT and WT opto-MTFs. hi, ≥2 Hz pacing; lo, <2 Hz pacing. High pacing rate and ISO increased re-entry occurrence. Pearson's chi-squared test vs WT with same conditions: †, P<0.05. ††, P<0.01. †††, P<0.001. Bars are labeled with sample numbers. FIGS. 3D-3F show the spatial maps of Ca²⁺ wave activation time, velocity, and CaTD80 in WT or CPVTp opto-MTFs. The same tissue is shown with 1.5 Hz or 3 Hz pacing. 3 Hz pacing increased spatiotemporal heterogeneity. FIGS. 3G-3H show the normalized global speed and CaTD80 (FIG. 3G) and their spatial and temporal dispersion (FIG. 3H) as a function of pacing frequency, under ISO stimulation. Data from tissues with 1:1 coupling were included (n=12 WT, 12 CPVTp, 13 CPVTe from >3 harvests). Smooth lines are quadratic functions fit to the data. Shaded areas represent the 95% confidence interval for the fit. In FIG. 3G, the data was normalized to values from the same opto-MTF at 1.5 Hz pacing without ISO. FIG. 3I shows the volcano plot shows 54 tissue-level parameters of Ca²⁺ propagation in WT vs. CPVT opto-MTFs. Each of the nine markers represents the indicated property measured at three different pacing rates (1, 2, and 3 Hz) with and without ISO. Shaded regions indicate parameters with P<0.05 and more than 2-fold change. Bar in A, D-F=1 mm.

FIGS. 4A-4D show the initiation of re-entry in CPVT opto-MTFs. FIG. 4A shows the CPVTp opto-MTF at 2 Hz pacing with ISO. The activation map and velocity fields were well-ordered. Speed histogram reflects narrow range of values. FIG. 4B shows the Ca²⁺ tracings from points a and b in panel FIG. 4A. FIG. 4C shows the same opto-MTF as in FIG. 4A, paced at 3 Hz with ISO. There is greater heterogeneity in the velocity field and disorganization of the activation map. Localized conduction block and retrograde conduction become evident at pulse #18 and #19. Histograms indicates greater spatial dispersion of speed. FIG. 4D shows the Ca²⁺ tracings at points a and b in panel FIG. 4C. The conduction block and initiation of re-entry was associated with a Ca²⁺ transient abnormality.

FIGS. 5A-5F show that CaMKII phosphorylation of RYR2-S2814 is required to express CPVT arrhythmic phenotype in isolated cell clusters. FIG. 5A shows the iPSC-CMs in isolated cell clusters were treated with ISO and selective CaMKII (C) or PKA (P) inhibitors. Ca²⁺ sparks were imaged by confocal line scanning. FIG. 5B shows the schematic of RYR2 (two subunits of tetramer shown). Three key residues are highlighted: S2808, the target of PKA phosphorylation; S2814, the target of CaMKII phosphorylation; and R4651, mutated in CPVTp. FIGS. 5C-5D show the Ca²⁺ spark frequency in CPVTe was reduced by S2814A but not S2808A mutation. FIG. 5C shows representative traces. FIG. 5D shows the distribution of Ca²⁺ spark frequency. FIGS. 5E-5F show abnormal Ca²⁺ transient frequency. Arrows and arrowheads indicate early and late abnormal Ca²⁺ release events in representative tracings (FIG. 5E). Distribution of the fraction of abnormal Ca²⁺ transients per cell (FIG. 5F). Steel-Dwass nonparametric test with multi-testing correction; †, vs WT with matching ISO treatment; §, vs CPVTe with matching ISO treatment. † or §, P<0.05; †† or §§, P<0.01; ††† or §§§, P<0.001.

FIGS. 6E-6H show that RYR2-S2814A mutation prevents re-entry in CPVT engineered tissues. FIG. 6A are confocal images of opto-MTF constructed using CPVTe-S2814A iPSC-CMs. Myocytes are aligned by micro-molded gelatin substrate. FIG. 6B shows representative CPVTe-S2814A opto-MTF. Ca²⁺ transients and systolic contraction were coupled 1:1 with 3 Hz optical stimuli (blue lines). FIG. 6C shows the occurrence of re-entry in CPVTe-S2814A compared to WT (†) and CPVTe (§) opto-MTFs under the matching conditions. Pearson's chi-squared test: † or §, P<0.05. †† or §§, P<0.01. ††† or §§§, P<0.001. Bars are labeled with samples sizes. FIG. 6D shows the spatial maps of the same CPVTe-S2814A opto-MTF paced at 1.5 Hz or 3.0 Hz, in the presence of ISO. Activation time, Ca²⁺ wave propagation speed, and CaTD80 were well-organized and relatively homogeneous compared to CPVTe (see FIG. 3). FIG. 6E-6F show the global speed and CaTD80 (FIG. 6E) or their spatial or temporal dispersion (FIG. 6F) as a function of pacing frequency in CPVTe-S2814A compared to CPVT and WT. Samples were treated with ISO. Samples were treated with ISO. Only tissues responding 1:1 to every stimulus were included (n=12 WT, 12 CPVTp, 13 CPVTe, and 18 CPVTe-52814A from >3 harvests). Smooth lines are quadratic functions fit to the data; shaded areas show the 95% confidence interval for the fit. Global speed and CaTD80 were normalized to data from the same tissue acquired at 1.5 Hz without ISO. FIG. 6G shows a volcano plot of 54 tissue-level parameters of Ca2+ wave propagation (please see FIG. 3). Unlike CPVT tissue parameters, CPVTe-S2814A tissue parameters were not statistically different from those of WT. FIG. 6H provides a schematic diagram illustrating an experimental strategy for generating adeno-associated virus (AAV) vectors encoding a CaMKII Inhibitory Peptide Autocamtide (AIP). AAV9 was injected into mice intraperitoneally an electrophysiology (EP) study.

FIG. 7 provides a series of panels showing the expression of AAV9-GFP-AIP in the heart (top row) and in micrographs of cardiac tissue.

FIG. 8 provides two graphs showing the percentage of cardiomyocytes infected by AAV9 viruses. The left graph shows cells with low GFP and cells with medium GFP signals. The column to the left of each pair of columns is GFP low and the column to the right is GFP medium. The right graph shows cells with different AIP therein, the columns in each 3 column set from left to right are AIP medium, high, and full. The first set of columns in each panel includes these identifiers.

FIG. 9 provides images of Western blots showing levels of phosphorylated (P) CaMKII vs. CaMKII (total) in whole heart lysates from p10 mice injected with an AIP expressing vector, AAV9-GFP-AIP, or with control vector.

FIG. 10 provides a box plot showing quantification of CaMKII phosphorylation in cells expressing AAV9-GFP-AIP or a control vector.

FIG. 11 provides a schematic diagram depicting a knock in of R176Q in the cardiac ryanodine channel (RYR2) as a model of CPVT.

FIG. 12 is a schematic diagram showing placement of a pacing and recording catheter in mice. The method is fully described in Mathur, N. et al. Circulation: Arrhythmia and Electrophysiology (2009).

FIG. 13 is a schematic diagram illustrating the protocal used to induce and record murine CVPT arrhythmias.

FIG. 14 shows baseline electrocardiograms in wild type and mice having an R176Q mutation in the cardiac ryanodine channel (RYR2).

FIG. 15 is a graph showing heart rate changes in wild-type (WT) and mice having a knock in of R176Q in the cardiac ryanodine channel (RYR2) where the mice are expressing an adenovirus encoding CaMKII Inhibitory Peptide Autocamtide (AIP) or a GFP control. The columns in each 3 column set from left to right are baseline, isoproterenol, and epinephrine. The first set of columns includes these identifiers.

FIG. 16 is a graph quantitating changes in QT interval. The columns in each 3 column set from left to right are baseline, isoproterenol, and epinephrine. The first set of columns includes these identifiers.

FIGS. 17A-17D are electrocardiograms showing baseline and spontaneous arrhythmia in mice having an R176Q mutation in the cardiac ryanodine channel (RYR2), (R176Q mutant mice) injected with GFP-expressing control vectors or injected with AIP-expressing vectors.

FIGS. 18A-18F show that in vivo expression of AIP reduces probability of induced arrhythmia with pacing (FIGS. 18A-18B) and catecholamines (FIGS. 18C-18D). FIG. 18E shows relative transduction level with increases doses of AAV9 viruses. FIG. 18F shows the suppression of induced ventricular arrhythmias with various doses of AIP. **P<0.001, § P=0.7. *P<0.01, †P=0.4. N as indicated. P-values by Chi-squared P-values by Chi-squared.

FIGS. 19A-19C CPVT patient with RYR2-R4651I mutation. FIG. 19A shows the electrocardiography data from an insertable cardiac monitoring system obtained for this patient. The patient developed bidirectional ventricular tachycardia (upper left), which converted into polymorphic ventricular tachycardia (upper right and lower left). The patient spontaneously recovered to a sinus rhythm (lower right). FIG. 19B shows the Sanger sequencing data at the RYR2-R4651 locus for a normal individual iPSCs and for a patient-derived iPSCs. Arrow points to point mutation that causes R4651I substitution. FIG. 19C is a schematic drawing of the RYR2 protein showing the mutation hotspot regions (Regions 1-4) and the location of the R4651I mutation within region 4.

FIGS. 20A-20G demonstrate the characterization and genome editing of CPVT iPSC lines. FIGS. 20A-20D show quality control analyses of the CPVTp iPSC line. CPVTp cells had normal karyotype (FIG. 20A), expression of pluripotency markers (FIG. 20B), typical colony morphology (FIG. 20C), and formed teratomas that produced derivatives from three germ layers, as assessed by H&E staining of histological sections (FIG. 20D). FIG. 20E is a schematic of the protocol used to differentiate iPSC-CMs from iPSCs. FIG. 20F is a FACS plot showing the purity of lactate-selected iPSC-CMs. FIG. 20G is Sanger sequencing results showing effective genome editing to introduce the R4651I heterozygous mutation into PGP1 wild-type iPSCs, creating the cell line named CPVTe.

FIG. 21 is the engineered Opto-MTF recording platform. Optical fibers stimulate focal areas on opto-MTF. Opto-MTF is illuminated under a microscope for simultaneous dark field imaging of mechanical cantilevers using a high spatial resolution camera, and fluorescent imaging of Ca²⁺ wave propagation using a high sensitivity, high speed camera

FIGS. 22A-22L show the fabrication of opto-MTFs seeded with hiPSC-CMs.

FIG. 23 shows the Ccnfocal image of CPVTe opto-MTF. CPVTe opto-MTF was immunostained for sarcomeric Z-disk marker ACTN2 and nuclear marker DAPI. iPSC-CMs were aligned in parallel. Bar=20 μm.

FIGS. 24A-24B show the optical mapping of Ca²⁺ wave propagation in an opto-MTF. FIG. 24A are time lapse images of opto-MTF showing X-Rhod-1 signal (“Ca²⁺ imaging”) and dark field imaging of deformable cantilevers at the terminus of the MTF. FIG. 24B are traces of Ca²⁺ transients and mechanical stress in MTFs. Ca²⁺ X-Rhod-1 signal was recorded at points a-d, labeled in the right-most image of (FIG. 24A). Vertical parallel lines across the traces indicate 488 nm optical pacing signals.

FIGS. 25A-25B show the independence of adjacent MTFs in opto-MTF construct. FIG. 25A shows peak systolic and diastolic contraction of MTFs upon independent optical stimulation on MTF with different pacing frequencies (1.5, 2, 3, and 4 Hz). FIG. 25B shows stress traces of each MTF. Each MTF is stimulated by a separate optical fiber at a different frequency. The mechanical systole of each MTF was independent of the other MTFs, as demonstrated here by the different frequencies of the stress traces. Blue lines indicate optical pacing.

FIGS. 26A-26D show the spatial and temporal dispersion of speed and calcium transient duration in opto-MTFs. Heterogeneity of propagation speed or calcium transient duration at 80% recovery (CaTD80) was calculated for opto-MTFs constructed using the indicated cells: NRVMs, neonatal rat ventricular cardiomyocytes; Cor.4U, iPSC-CMs from Axiogenesis; WT, CPVTe, and CPVTe-S2814A, iPSC-CMs from this study. Name Statistical test: *, P<0.05. **, P<0.01. ***, P<0.001.

FIGS. 27A-27D show the spontaneous Ca²⁺ waves in opto-MTFs. Opto-MTFs constructed from CPVTp (FIG. 27A), CPVTe (FIG. 27B), or WT iPSC-CMs (FIG. 27C), allowed to beat spontaneously. Ca²⁺ waves were optically recorded by X-Rhod-1 fluorescence intensity. In FIGS. 27A-27C, the left panels are activation maps, and right traces are Ca²⁺ signal at indicated points on the MTF. Note lack of aberrant Ca²⁺ transients. Spontaneous Ca²⁺ waves originated from the edges of the MTFs. FIG. 27D shows the Ca²⁺ signal at individual pixels of the indicated tissues were analyzed for Ca²⁺ transient abnormalities consistent with EADs or DADs. None were observed in any of the spontaneously beating opto-MTFs. The spontaneous beating frequency of the opto-MTFs was comparable between iPSC-CM types. All represents the union of all CPVTp and CPVTe tissues recorded.

FIG. 28 demonstrates the occurrence of re-entry in WT, CPVTp, and CPVTe opto-MTFs. Occurrence of re-entry in opto-MTFs assembled from WT, CPVTp, and CPVTe opto-MTFs, stimulated with ISO or low (<2 Hz) or high (≥2 Hz) pacing. \, comparison to comparable treatment group for WT; § Comparison to comparable treatment group for CPVTp. Fisher test: † or §, P<0.05; \\ or §§, P<0.01; \\\, or §§§, P<0.001.

FIGS. 29A-29D show the reentry in CPVTe opto-MTF. FIGS. 29A-29B. Pacing at 2 Hz. Ca²⁺ waves are well-ordered. Ca²⁺ traces from points labeled in left panel of A are shown in B. FIGS. 29C-29D. Pacing of the same tissue at 3 Hz. Ca²⁺ waves are chaotic, and multiple areas of reentry form. Ca²⁺ traces from points labeled in left panels of FIG. 29C are shown in FIG. 29D. Note that the most distal point d has 3:2 or 2:1 coupling with the pacing stimulus. Activation maps and Ca²⁺ traces were calculated by processing Ca²⁺ imaging data in movies obtained.

FIGS. 30A-30B show the vulnerability of WT, CPVTp, and CPVTe opto-MTFs to re-entry. FIG. 30A shows the Ca²⁺ wave propagation speed and CaTD80 of ISO-treated tissues at indicated pacing rates. FIG. 30B shows the spatial and temporal dispersion of Ca²⁺ wave propagation speed and CaTD80 in ISO-treated tissues at indicated pacing rates.

FIG. 31 shows the statistical analysis of opto-MTF properties. Nine parameters were analyzed with and without ISO treatment at 1, 2, and 3 Hz pacing frequencies. These 54 comparisons were made between WT and CPVT (union of CPVTp and CPVTe) and between WT and CPVTe-S2814A.

FIGS. 32A-32D show the initiation of re-entry in CPVTe opto-MTF. FIGS. 32A-32B show the organized Ca²⁺ waves at 2 Hz pacing. Traces in FIG. 32B were recorded from points labeled in FIG. 32A. FIGS. 32C-32D. Development of re-entry at 2.5 Hz pacing. Traces in FIG. 32D were recorded from points labeled in FIG. 32C. Note the development of Ca²⁺ transient abnormality following pulse 2, accompanied by re-entry initiation at pulse 3.

FIG. 33 shows the inhibition of CaMKII activity by cell permeable inhibitory peptide. iPSCCMs were treated with the cell permeable CaMKII peptide inhibitor AIP (250 nM). Cells were stimulated for 60 minutes with 1 μM ISO prior to analyzing cell extracts by immunoblotting. In wild-type (PGP1) cells, CaMKII T286 phosphorylation was blocked by AIP, while total CaMKII was unchanged. In CPVTp cells, there was basal activation of CaMKII. This was blocked by AIP.

FIGS. 34A-34D show the genome editing of S2808 and S2814 sites of RYR2. FIG. 34A is a schematic of the genome editing strategy used to obtain homozygous S2808A or S2814A mutations in either PGP1 (WT) or PGP1-RYR2R4651I/+(CPVTe) iPSCs. FIG. 34B shows representative Sanger sequencing to confirm genome editing. FIG. 34C shows iPSC-CM differentiation of genome edited cell lines. FIG. 34D shows S2814A mutant cell lines did not exhibit S2814 phosphorylation on ISO stimulation.

FIG. 35A shows the CPVT patients have normal resting electrocardiograms but severe, potentially life-threatening arrhythmias with exercise. VT, ventricular tachycardia. VF, ventricular fibrillation. Traces are idealized sketches shown for illustration purposes.

FIG. 35B shows the CPVT pathophysiology. Left, cartoon of cardiomyocyte Ca²⁺-induced Ca²⁺ release. 1. Action potential opens L-type Ca²⁺ channel (LTCC); 2. Ca²⁺ induces opening of RYR2 and release of Ca²⁺ from the sarcoplasmic reticulum (SR); 3. Elevated intracellular Ca²⁺ induces myofilament contraction; 4. Ca²⁺ is cleared from the cytosol by SERCA and NCX. Right, CPV mutations in RYR2 increase diastolic Ca²⁺ leak.

FIG. 36 shows a schematic for treatment of adolescent animals with AAV9 and workflow for testing of single cells and with ventricular pacing.

FIGS. 37A-37B show the effects of AAV9-GFP-AIP on single isolated cardiomyocytes from treated animals. FIG. 37A demonstrates confocal line tracings of Ca²⁺ indicator (Rhod-2) after external pacing for 1 minute. Spontaneous Ca²⁺ is recorder and quantified (FIG. 37B). N=33 (GFP), N=25 (AIP). **P<0.01.

FIGS. 38A-38C show the suppression of induced ventricular arrhythmias in R176Q mutant mice treated with either GFP or AIP by AAV9 by retro-orbital injection. FIG. 38A shows representative tracing of induced ventricular arrhythmia (top panel) or no arrhythmia (bottom panel) in either GFP or AIP treated animals respectively. FIGS. 38B-38C show the percent of animals with ventricular arrhythmias (FIG. 38B) or duration of ventricular arrhythmias induced by pacing (FIG. 38C). N=6 (GFP), N=6 (AIP), *P<0.01.

FIGS. 39A-39B show suppression of abnormal Ca²⁺ signaling with modified RNAs to peptide inhibitors. Adult cardiomyocytes from RYR2-R176Q mice were transfected with modified RNA (modRNAs) for peptide inhibitors AIP and CN190 or fused to RYR2 binding protein FKBP12.6 and co-expressing mCherry. After culturing for 12 hours, individual cardiomyocytes were loaded with a Ca²⁺ indicator (Fluo-4) and paced for 1 minute prior to recording of post-pacing Ca²⁺ events. FIG. 39A shows representative confocal line tracings of adult cardiomyocytes and expression of mCherry (left). FIG. 39B shows quantification of all tested inhibitors compared to mCherry only. Statistically significant reduction in abnormal post-pacing Ca²⁺ events in cardiomyocytes expressing CN190 and near significant in cells expressing AIP as compared to mCherry only. N=15 (mCherry), N=5 (AIP), N=5 (FKBP12.6-AIP), N=5 (CN19o), N=7 (FKBP12.6-CN19o). P-value for AIP=0.07, *P<0.05, § P>0.5.

FIG. 40 shows relative expression of novel AAV capsids across multiple tissues. After injection of 2×10¹⁰ vg/g of each AAV virus by subcutaneous injection at post-natal day 3, tissues were harvested at post-natal day 28 and processed for total RNA. FIG. 40 shows relative GFP mRNA levels normalized to expression of tata-binding protein (TBP). Self-complementary (SC) Anc82 demonstrates increased expression in muscle and heart as compared to AAV9.

FIG. 41 shows AIP inhibition of aberrant calcium transients in two additional patient-derived iPSC-CMs containing RYR2 mutations in hotspot regions 1 and 3 (R1 and R3). CPVT-R1 and CPVT-R3 genotypes were S404R and G3946S respectively. AIP was effective in reducing abnormal calcium transients in these additional two CPVT genotypes. Number of individual cells as indicated, P<0.01 by Chi-Squared.

FIG. 42 shows AIP inhibition of aberrant calcium sparks in Cas9-engineered iPSC-CMs (CPVTe2) that are otherwise isogenic to the WT line. The engineered mutation is RYR2-D385N, which is found in CPVT patients. AIP reduced calcium spark frequency back to rates comparable to those seen in WT.

### DETAILED DESCRIPTION OF THE INVENTION

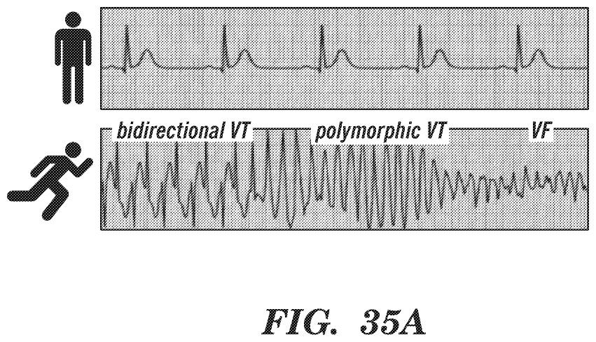

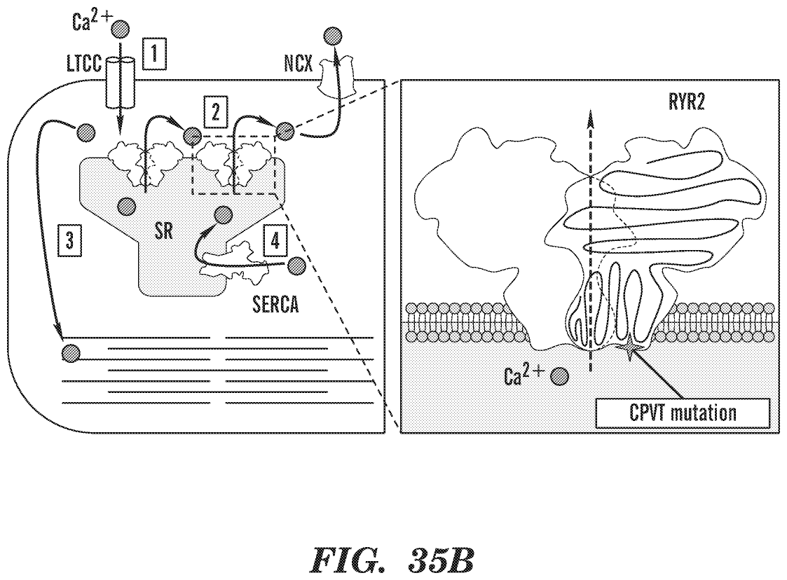

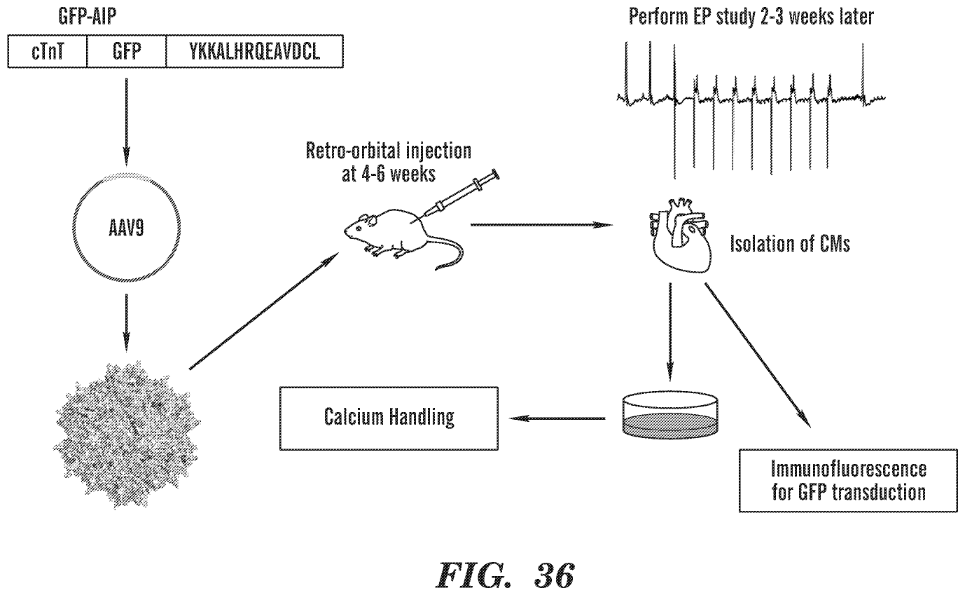

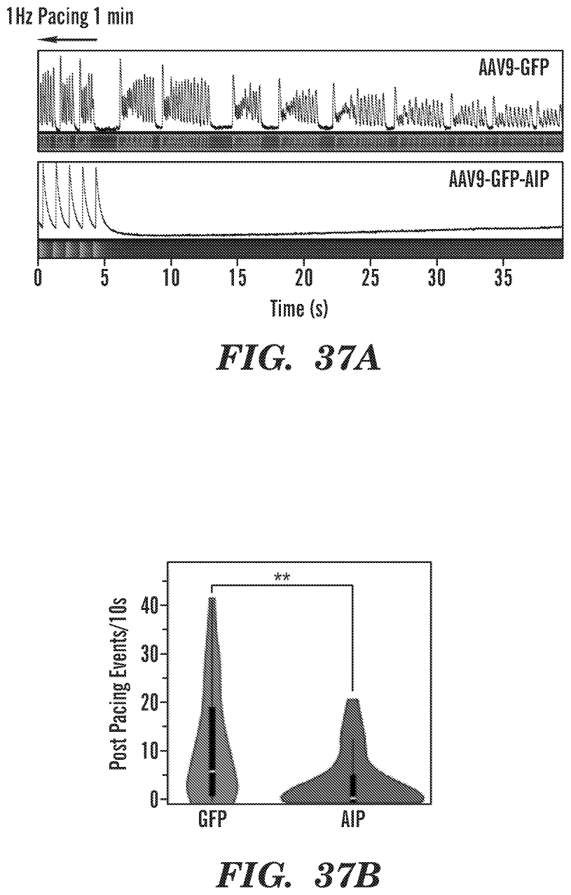

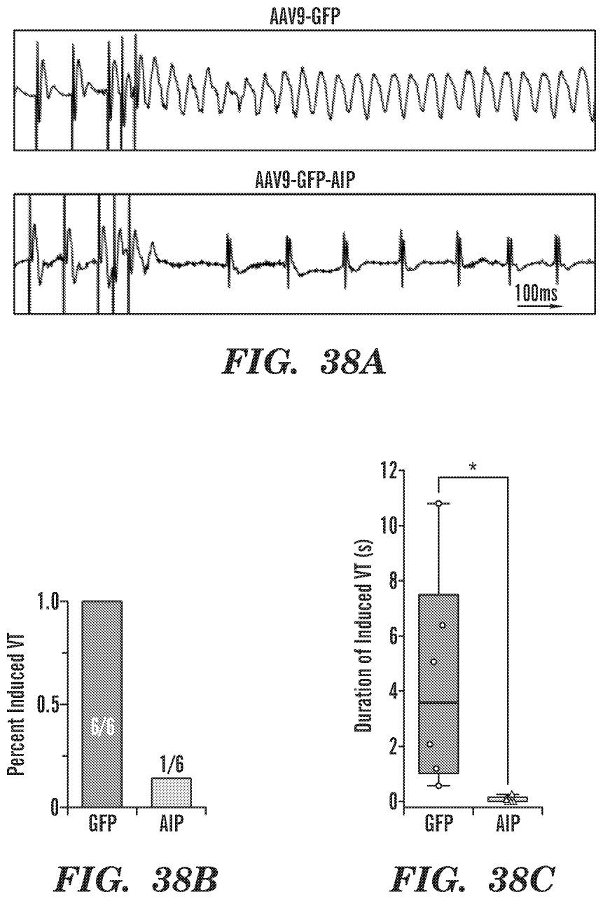

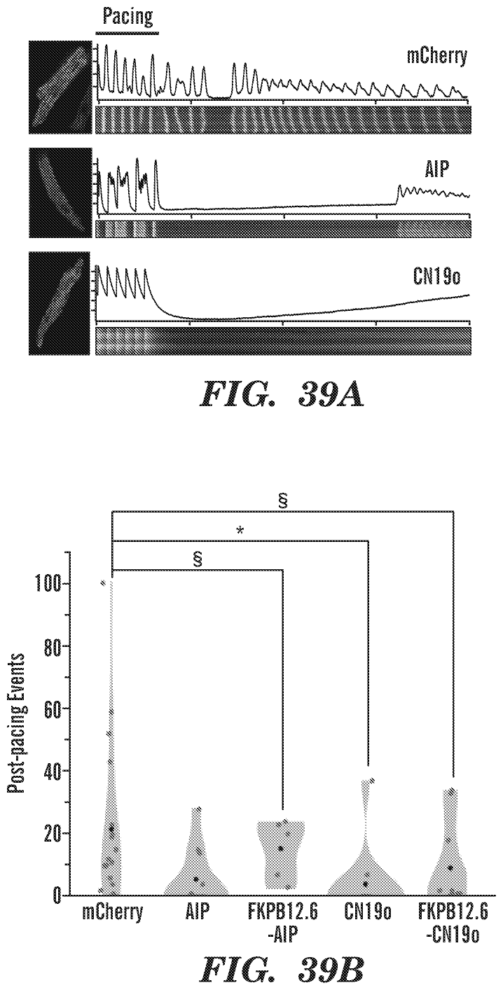

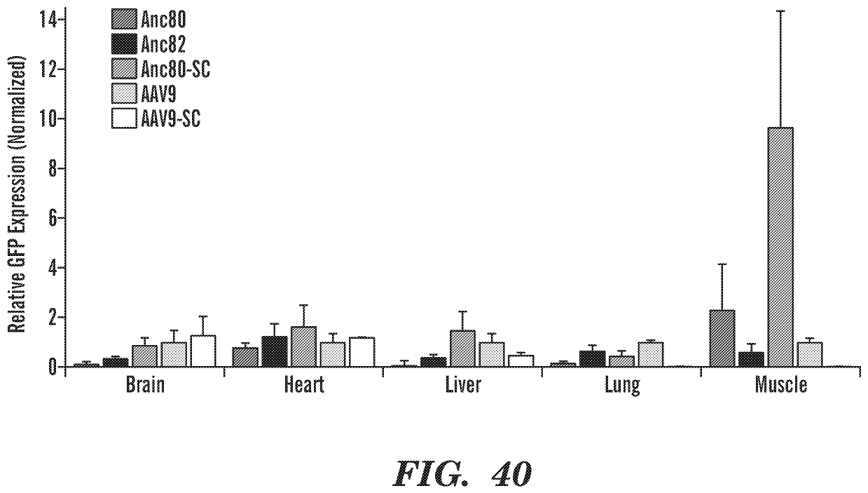

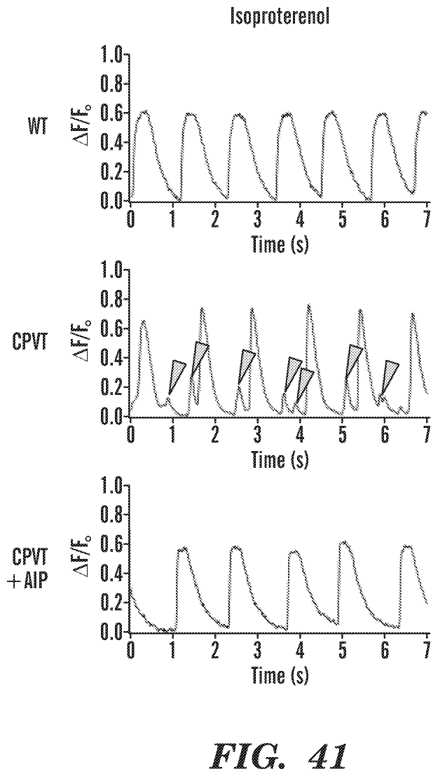

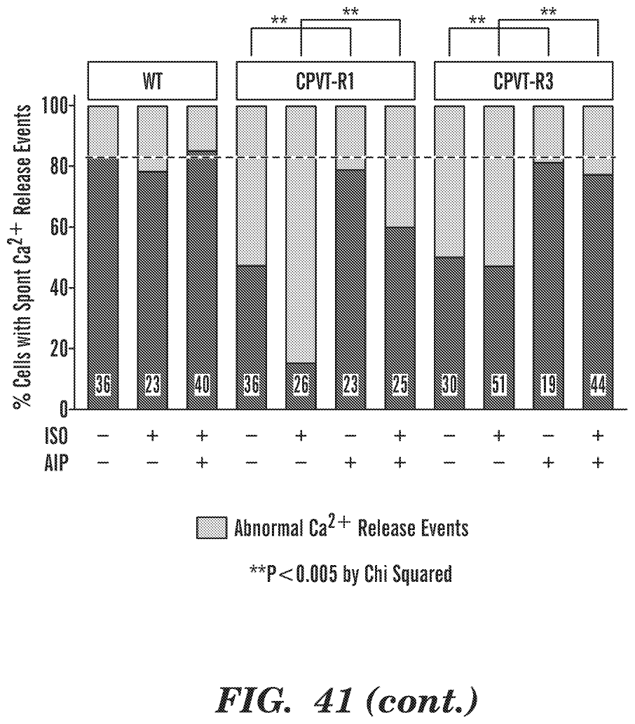

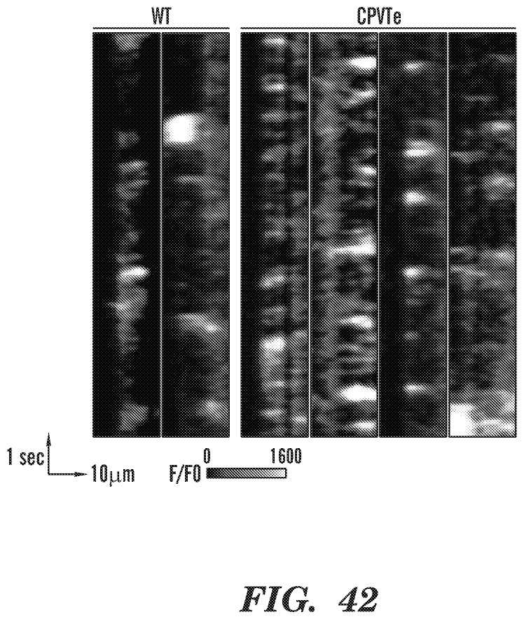

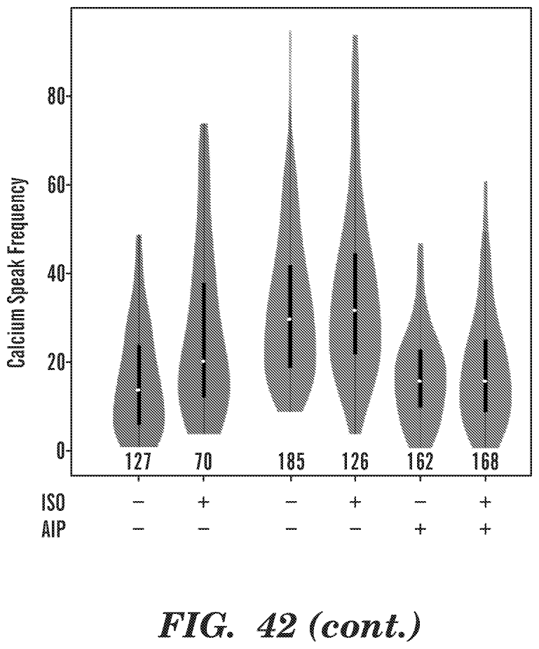

Catecholaminergic polymorphic ventricular tachycardia (CPVT) is an inherited arrhythmia predominantly caused by autosomal dominant mutation of the gene encoding the cardiac ryanodine receptor (RYR2), the main intracellular calcium release channel of cardiomyocytes. Typically, CPVT patients are asymptomatic at rest but develop potentially lethal ventricular tachycardia during exercise or emotional distress (FIG. 35A). In wild type cardiomyocytes, when the cardiac action potential opens the voltage sensitive L-type Ca²⁺ channel located in the plasma membrane, the resulting local influx of Ca²⁺ triggers release of Ca²⁺ from the sarcoplasmic reticulum via RYR2 (FIG. 35B). The resulting increase in cytoplasmic Ca²⁺ leads to sarcomere contraction. As the cell enters diastole, RYR2 closes and cytosolic Ca²⁺ is pumped back into the sarcoplasmic reticulum. In cells carrying mutations associated with CPVT, RYR2 releases more into the cytoplasm, resulting in elevated diastolic Ca²⁺ that drives exchange of sodium and calcium through the plasma membrane via the sodium calcium exchanger (NCX1), leading to after-depolarizations that may trigger additional action potentials. The molecular mechanism by which catecholamine stimulation unmasks the arrhythmic nature of CPVT mutations is not known. The mechanisms by which RYR2 mutation yields the clinical phenotype of ventricular tachycardia is also uncertain.

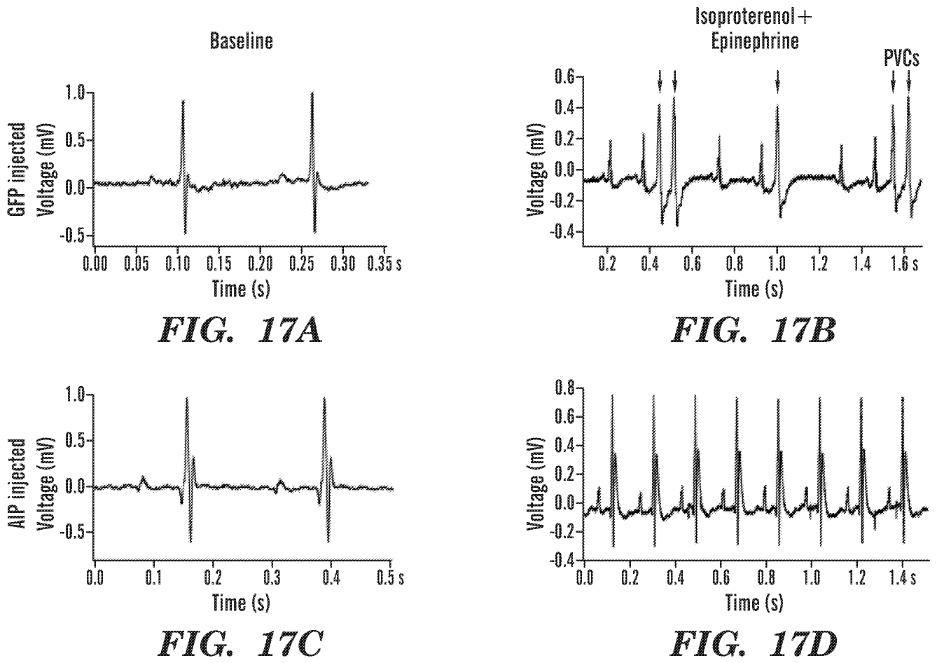

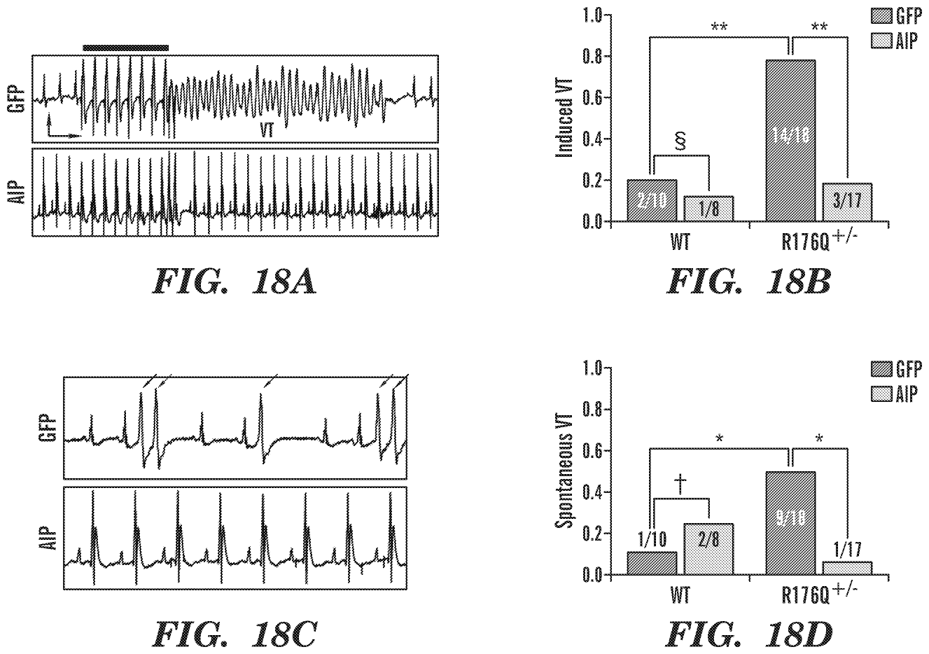

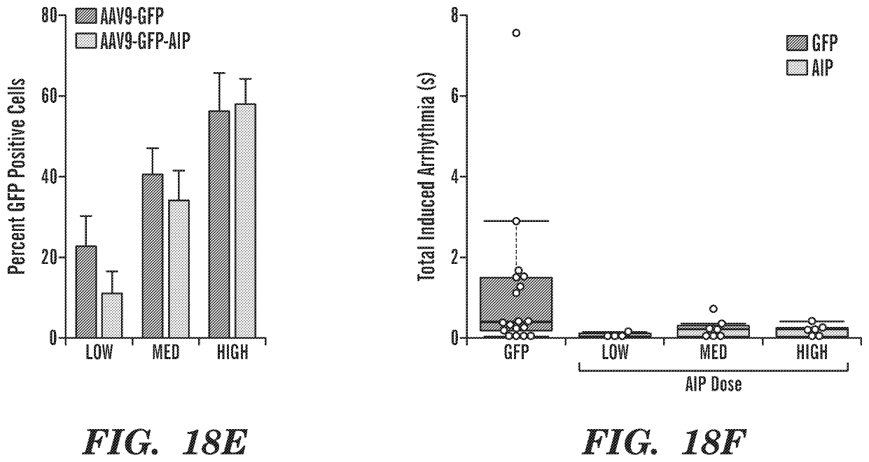

The inventors discovered that the inhibition of CaMKII activation and subsequent downstream signaling significantly reduces the catecholamine-stimulated latent arrhythmia that is associated with mutations in the calcium ryanodine channel, RYR2. In in vivo experiments, the inventors showed that the peptide inhibitor, AIP, when expressed in vivo in cardiac tissues of CPVT model mice, inhibited arrhythmia in the CPVT model mice. See Example 2, FIGS. 17 and 18. The inventors also found that the CaMKII-mediated phosphorylation of the serine residue at S2814 in RYR2 is essential for catecholamine-stimulated latent arrhythmic in CPVT mutations. Mutation of the serine to alanine reverses the aberrant Ca²⁺ spark frequency recorded for cardiac cells having CPVT-associated mutations in the RYR2 protein.

Accordingly, the invention features compositions featuring CAMKII inhibitors, such as an AIP peptide, analog, or fragment thereof, polynucleotides encoding such peptides, therapeutic compositions comprising AIP peptides and polynucleotides, and methods of using such compositions for the treatment of subjects having a mutation in a cardiac ryanodine channel (RYR2) that predisposes them to CPVT. These peptide inhibitors may be delivered using adeno-associated viral (AAV) vectors, or other vectors including adenovirus, and lentivirus. The compositions and methods may also be used for treatment of other forms of cardiac disease. Whereas isolated CPVT iPSC-derived cardiomyocytes (iPSC-CMs) were prone to aberrant calcium transients, these were uncommon in unstimulated CPVT tissues. However, CPVT tissues stimulated by catecholamines and rapid pacing were vulnerable to action potential re-entry, recapitulating the hallmark exercise-dependence of the clinical disease. Using Cas9 genome editing, a single catecholamine-driven phosphorylation event, RYR2-S2814 phosphorylation by Ca²⁺-calmodulin-dependent protein kinase II (CaMKII), was identified as required to unmask pro-arrhythmia in engineered CPVT myocardial sheets. These studies illuminate the molecular and cellular pathogenesis of CPVT, revealing a critical role of CaMKII-dependent re-entry in the tissue-scale mechanism of this disease. Importantly, the invention provides an in vitro arrhythmia model comprising iPSC-CMs in an engineered, optogenetic myocardial tissue model.

In one aspect, provided herein is a pharmaceutical composition comprising an effective amount of a vector encoding a CaMKII peptide inhibitor and a pharmaceutically acceptable carrier.

In one aspect, provided herein is a pharmaceutical composition comprising an effective amount of a vector encoding a CaMKII peptide inhibitor and a pharmaceutically acceptable carrier for use in the treatment of cardiac arrhythmia, for example, such as CPVT.

In one aspect, provided herein is a pharmaceutical composition comprising an effective amount of a vector encoding a CaMKII peptide inhibitor and a pharmaceutically acceptable carrier for use in the manufacture of medicament for the treatment of cardiac arrhythmia, for example, such as CPVT.

In another aspect, provided herein is an expression vector comprising a polynucleotide encoding a CaMKII peptide inhibitor.

In one aspect, provided herein is an expression vector comprising a polynucleotide encoding a CaMKII peptide inhibitor for use in the treatment of cardiac arrhythmia, for example, such as CPVT.

In one aspect, provided herein is an expression vector comprising a polynucleotide encoding a CaMKII peptide inhibitor for use in the manufacture of medicament for the treatment of cardiac arrhythmia, for example, such as CPVT.

In another aspect, provided herein is a cell comprising an expression vector comprising a polynucleotide encoding a CaMKII peptide inhibitor.

In one aspect, provided herein is a cell comprising an expression vector comprising a polynucleotide encoding a CaMKII peptide inhibitor for use in the treatment of cardiac arrhythmia, for example, such as CPVT.

In one aspect, provided herein is a cell comprising an expression vector comprising a polynucleotide encoding a CaMKII peptide inhibitor for use in the manufacture of medicament for the treatment of cardiac arrhythmia, for example, such as CPVT.

The described a pharmaceutical composition, expression vector, and cells comprising an expression vector are all useful for the treatment of cardiac arrhythmia in a subject.

In another aspect, provided herein is a method for modulating a cardiac arrhythmia in a subject, the method comprising contacting a cell comprising a cardiac ryanodine channel (RYR2) with a CaMKII inhibitor, CaMKII peptide inhibitor or polynucleotide encoding the CaMKII peptide inhibitor.

In another aspect, provided herein is a method for inhibiting the phosphorylation of a ryanodine channel (RYR2) polypeptide in a subject, the method comprising contacting a cell comprising a cardiac ryanodine channel (RYR2) with a CAMKII inhibitor, CaMKII peptide inhibitor or polynucleotide encoding a CaMKII peptide inhibitor.

In another aspect, provided herein is a method of treating a subject comprising a mutation associated with a cardiac arrhythmia, the method comprising administering to the subject a CaMKII inhibitor, CaMKII peptide inhibitor, analog, or fragment thereof or polynucleotide encoding a CaMKII peptide inhibitor.

In another aspect, provided herein is a method of treating a subject having a cardiac arrhythmia, the method comprising administering to the subject a pharmaceutical composition comprising an effective amount of a vector encoding a CaMKII peptide inhibitor and a pharmaceutically acceptable carrier. In one embodiment, the cardiac arrhythmia is CPVT. In one embodiment, the pharmaceutical composition is administered intravenously or by intracardiac injection.

In another aspect, provided herein is a method of characterizing a cardiomyocyte, the method comprising monitoring cardiac conduction or contraction using an induced pluripotent stem cell derived cardiomyocyte (iPSC-CM) expressing a cardiac ryanodine channel (RYR2) comprising a mutation associated with CPVT.

In another aspect, provided herein is a method of compound screening, the method comprising contacting an induced pluripotent stem cell derived cardiomyocyte expressing a cardiac ryanodine channel (RYR2) comprising a mutation associated with CPVT with a candidate agent and measuring cardiac conduction or contraction in the cell. In other embodiments, the method comprises measuring Ca²⁺ spark frequency and Ca²⁺ re-entry and other parameters described in the Example section.

In one embodiment of any one aspect described, the CaMKII peptide inhibitor is AIP, CN19, CN19o, CN27, CN21, or an analog or fragment thereof.

In one embodiment of any one aspect described or any one prior embodiment described, the CaMKII peptide inhibitor is operably linked to a promoter suitable for driving expression of the peptide in a mammalian cardiac cell. Promoters for cardiac muscle cell-specific expression are known in the art, for examples, the cardiac troponin T promoter, the α-myosin heavy chain (α-MHC) promoter, the myosin light chain-2v (MLC-2v) promoter or the cardiac NCX1 promoter.

In one embodiment of any one aspect of the method described, the contacted cell is a cardiomyocyte.

In one embodiment of any one aspect of the method described or any one prior embodiment described, the contacted cardiomyocyte has a mutation in a cardiac ryanodine channel (RYR2) therein.

In one embodiment of any one aspect of the method described or any one prior embodiment described, the contacted cardiomyocyte has more than one mutation in a RYR2 channel therein.

In one embodiment of any one aspect described or any one prior embodiment described, the vector is used in a pharmaceutical composition comprising an effective amount of an CaMKII peptide inhibitor, analog, or fragment thereof.

In one embodiment of any one aspect described or any one prior embodiment described, the vector is a retroviral, adenoviral, or adeno-associated viral vector.

In one embodiment of any one aspect described or any one prior embodiment described, the cardiac arrhythmia is a ventricular tachycardia.

In one embodiment of any one aspect described or any one prior embodiment described, the ventricular tachycardia is exercise-induced or stress-induced.

In one embodiment of any one aspect described or any one prior embodiment described, the ventricular tachycardia is CPVT.

In one embodiment of any one aspect described or any one prior embodiment described, the cardiac arrhythmia involves or is associated with a genetic mutation.

In one embodiment of any one aspect described or any one prior embodiment described, the genetic mutation associated with the cardiac arrhythmia is found in a RYR2 channel in the cardiomyocytes.

In one embodiment of any one aspect described or any one prior embodiment described, the genetic mutation in RYR2 occurs in region 1 (amino acid residues 77-466), region 2 (amino acid residues 2246-2534), region 3 (amino acid residues 3778-4201) or region 4 (amino acid residues 4497-4959) of the RYR2 polypeptide.

In one embodiment of any one aspect described or any one prior embodiment described, the genetic mutation in RYR2 is an amino acid arginine to isoleucine substitution at the amino acid position 4651 in region 4 of the RYR2 polypeptide (R4651I) (RYR2^{R4651I}).

In one embodiment of any one aspect described or any one prior embodiment described, the genetic mutation in RYR2 is an amino acid arginine to glutamine substitution at the amino acid position 176 in region 1 of the RYR2 polypeptide (R176Q) (RYR2 ^{R176Q}).

In one embodiment of any one aspect described or any one prior embodiment described, the genetic mutation in RYR2 is an amino acid aspartic acid to asparagine substitution at the amino acid position 385 of the RYR2 polypeptide (D385N) (RYR2^{D385N}).

In one embodiment of any one aspect described or any one prior embodiment described, the genetic mutation in RYR2 is an amino acid serine to arginine substitution at the amino acid position 404 of the RYR2 polypeptide (S404R) (RYR2^{S404R}).

In one embodiment of any one aspect described, the genetic mutation in RYR2 is an amino acid glycine to serine substitution at the amino acid position 3946 of the RYR2 polypeptide (G3946S) (RYR2^{G3946S}).

In one embodiment of any one aspect of the method described or any one prior embodiment described, the method inhibits a cardiac arrhythmia in the subject.

In one embodiment of any one aspect of the method described or any one prior embodiment described, the method reduces the incidences of cardiac arrhythmia in the subject. For example, the frequency of cardiac arrhythmia over a period of time in the subject.

In one embodiment of any one aspect of the method described or any one prior embodiment described, the method reduces the incidences of cardiac arrhythmia in the subject during exercise stimulation or emotional stress.

In one embodiment of any one aspect of the method described or any one prior embodiment described, the method inhibits catecholaminergic polymorphic ventricular tachycardia (CPVT) in the subject.

In one embodiment of any one aspect of the method described or any one prior embodiment described, the method reduces CPVT in the subject.

In one embodiment of any one aspect of the treatment or modulation method described or any one prior embodiment described, the method further comprises selecting a subject having a cardiac arrhythmia or CPVT.

In one embodiment of any one aspect of the treatment or modulation method described or any one prior embodiment described, the method further comprises selecting a subject having a mutation associated with a cardiac arrhythmia or CPVT.

In one embodiment of any one aspect of the treatment or modulation method described or any one prior embodiment described, the method further comprises selecting a subject having a mutation associated with a cardiac arrhythmia, wherein the mutation is found in a calcium ryanodine channel (RYR2) in the cardiomyocytes. In one embodiment, the genetic mutation in RYR2 occurs in region 1 (amino acid residues 77-466), region 2 (amino acid residues 2246-2534), region 3 (amino acid residues 3778-4201) or region 4 (amino acid residues 4497-4959) of the RYR2 polypeptide. In another embodiment, the genetic mutation is selected from the group consisting of RYR2^{R4651I}, RYR2^{R176Q}, RYR2^{D385N}, RYR2^{S404R}, and RYR2^{G3946S}.

In one embodiment of any one aspect of the screening method described or any one prior embodiment described, the iPSC-CM is derived from a subject having a mutation associated with a cardiac arrhythmia. In another embodiment, the subject has more than one mutation associated with a cardiac arrhythmia, such as RYR2^{R4651I} RYR2^{R176Q}, RYR2^{D385N}, RYR2^{S404R}, and RYR2^{G3946S} in the RYR2 channel protein.

In one embodiment of any one aspect of the screening method described or any one prior embodiment described, the iPSC-CM has one or more mutation in a cardiac ryanodine channel (RYR2) therein. For examples, having both RYR2^{R4651I} and RYR2^{R176Q} mutations, both RYR2^{D385N} and YUR2^{S404R} mutations, or both RYR2^{S404R} and RYR2^{G3946S} mutations. In some embodiments, all possible combinations of multiple mutations occurring at RYR2^{R4651I}, RYR2^{R176Q}, RYR2^{D385N}, RYR2^{S404R}, and RYR2^{G3946S} in the RYR2 channel protein are included.

In one embodiment of any one aspect of the screening method described or any one prior embodiment described, the iPSC-CM has one or more mutation in RYR2 occurs in region 1 (amino acid residues 77-466), region 2 (amino acid residues 2246-2534), region 3 (amino acid residues 3778-4201) or region 4 (amino acid residues 4497-4959) of the RYR2 polypeptide.

In one embodiment of any one aspect of the screening method described or any one prior embodiment described, the iPSC-CM has a mutation that results in an amino acid arginine to isoleucine substitution at the amino acid position 4651 in region 4 of the RYR2 polypeptide (R4651I) (RYR2^{R4651I}).

In one embodiment of any one aspect of the screening method described or any one prior embodiment described, the iPSC-CM has a mutation results in an amino acid arginine to glutamine substitution at the amino acid position 176 in region 1 of the RYR2 polypeptide (R176Q) (RYR2^{R176Q}).

In one embodiment of any one aspect of the screening method described or any one prior embodiment described, the iPSC-CM has a mutation results in an amino acid aspartic acid to asparagine substitution at the amino acid position 385 of the RYR2 polypeptide (D385N) (RYR2^{D385N})

In one embodiment of any one aspect of the screening method described or any one prior embodiment described, the iPSC-CM has a mutation results in an amino acid serine to arginine substitution at the amino acid position 404 of the RYR2 polypeptide (S404R) (RYR2^{S404R}).

In one embodiment of any one aspect of the screening method described or any one prior embodiment described, the iPSC-CM has a mutation results in an amino acid glycine to serine substitution at the amino acid position 3946 of the RYR2 polypeptide (G3946S) (RYR2^{G3946S}).

In one aspect, provided herein is an induced pluripotent stem cell derived cardiomyocyte (iPSC-CM) expressing a cardiac ryanodine channel (RYR2) comprising a mutation associated with CPVT. For example, such as RYR2^{R4651I} , RYR2^{R176Q}, RYR2^{D385N}, RYR2^{S404R}, and RYR2^{R3946S}.

In one aspect, provided herein is an induced pluripotent stem cell derived cardiomyocyte (iPSC-CM) expressing a cardiac ryanodine channel (RYR2) comprising a mutation therein. In another aspect, provided herein is a composition comprising iPSC-CMs expressing a cardiac ryanodine channel (RYR2) comprising a mutation therein.

In another aspect, provided herein is a composition comprising iPSC-CMs expressing a cardiac ryanodine channel (RYR2) comprising a mutation associated with CPVT. For example, such as RYR2^{R4651I}, RYR2^{R176Q}, RYR2^{D385N}, RYR2^{S404R}, and RYR2^{G3946S}.

In one embodiment of any one aspect of the iPSC-CM described or composition comprising the iPSC-CM described or any one prior embodiment described, the iPSC-CM has a mutation in RYR2 occurs in region 1 (amino acid residues 77-466), region 2 (amino acid residues 2246-2534), region 3 (amino acid residues 3778-4201) or region 4 (amino acid residues 4497-4959) of the RYR2 polypeptide.

In one embodiment of any one aspect of the iPSC-CM described or composition comprising the iPSC-CM described or any one prior embodiment described, the iPSC-CM has more than one mutation in RYR2 channel.

In one embodiment of any one aspect of the iPSC-CM described or composition comprising the iPSC-CM described or any one prior embodiment described, the iPSC-CM has a mutation that results in an amino acid arginine to isoleucine substitution at the amino acid position 4651 in region 4 of the RYR2 polypeptide (R4651I) (RYR2^{R4651I}).

In one embodiment of any one aspect of the iPSC-CM described or composition comprising the iPSC-CM described or any one prior embodiment described, the iPSC-CM has a mutation results in an amino acid arginine to glutamine substitution at the amino acid position 176 in region 1 of the RYR2 polypeptide (R176Q) (RYR2^{R176Q}).

In one embodiment of any one aspect of the iPSC-CM described or composition comprising the iPSC-CM described or any one prior embodiment described, the iPSC-CM has a mutation results in an amino acid aspartic acid to asparagine substitution at the amino acid position 385 of the RYR2 polypeptide (D385N) (RYR2^{D385N}).

In one embodiment of any one aspect of the iPSC-CM described or composition comprising the iPSC-CM described or any one prior embodiment described, the iPSC-CM has a mutation results in an amino acid serine to arginine substitution at the amino acid position 404 of the RYR2 polypeptide (S404R) (RYR2^{S4041}).

In one embodiment of any one aspect of the iPSC-CM described or composition comprising the iPSC-CM described or any one prior embodiment described, the iPSC-CM has a mutation results in an amino acid glycine to serine substitution at the amino acid position 3946 of the RYR2 polypeptide (G3946S) (RYR2^{G3946S}).

In one embodiment of any one aspect of the iPSC-CM described or composition comprising the iPSC-CM described or any one prior embodiment described, the iPSC-CM has a mutation in RYR2 at S2814. For example, a S2814A mutation.

In one embodiment of any one aspect of the iPSC-CM described or composition comprising the iPSC-CM described or any one prior embodiment described, the iPSC-CM has a mutation in RYR2 at S2808. For example, a S2808A mutation.

In one embodiment of any one aspect of the iPSC-CM described or composition comprising the iPSC-CM described or any one prior embodiment described, the iPSC-CM has a first mutation in RYR2 at S2808 or S2814, and a second mutation in RYR2 that occurs in region 1 (amino acid residues 77-466), region 2 (amino acid residues 2246-2534), region 3 (amino acid residues 3778-4201) or region 4 (amino acid residues 4497-4959) of the RYR2 polypeptide. For example, a first mutation in RYR2 at S2808 or S2814, and a second mutation at R4651 or R176.

In one embodiment of any one aspect of the iPSC-CM described or composition comprising the iPSC-CM described or any one prior embodiments described, the mutation is an amino acid substitution. For example, a serine to alanine substitution, or an arginine to glutamine substitution, or an arginine to isoleucine substitution.

In one embodiment of any one aspect of the composition comprising the iPSC-CM described or any one prior embodiments described, the composition further comprises a pharmaceutically acceptable carrier.

Catecholaminergic Polymorphic Ventricular Tachycardia (CPVT)

Catecholaminergic polymorphic ventricular tachycardia (CPVT) is an inherited arrhythmia predominantly caused by autosomal dominant mutation of the gene encoding the cardiac ryanodine receptor 2 (RYR2), the main intracellular calcium release channel of cardiomyocytes. Typically, CPVT patients are asymptomatic at rest but develop potentially lethal ventricular tachycardia during exercise or emotional distress. The cardiac action potential opens the voltage sensitive L-type Ca²⁺ channel located in the plasma membrane. The resulting local influx of Ca²⁺ opens RYR2, positioned on the sarcoplasmic reticulum, releasing Ca²⁺ into the cytosol where it triggers sarcomere contraction. When the cardiac action potential ends and the cell enters diastole, RYR2 closes and cytosolic Ca²⁺ is pumped back into the sarcoplasmic reticulum by the sarcoplasmic reticulum Ca²⁺-ATPase.

CPVT mutations increase diastolic Ca²⁺ release from the sarcoplasmic reticulum into the cytoplasm by RYR2. In individual cardiomyocytes, elevated diastolic Ca²⁺ induces reverse sodium-calcium exchange through NCX1 at the plasma membrane, resulting in after-depolarizations that potentially can trigger additional action potentials. The molecular mechanism by which catechol stimulation unmasks the arrhythmic nature of CPVT mutations is not known, although catechol-induced activation of Ca²⁺-calmodulin-dependent protein kinase II (CaMKII) has been implicated. The mechanisms by which RYR2 mutation yields the clinical phenotype of ventricular tachycardia is also uncertain, although one theory is that cardiomyocyte triggered activity produces ventricular tachycardia.

The advent of induced pluripotent stem cell (iPSC) technology and efficient methods to differentiate iPSCs to cardiomyocytes (iPSC-CMs) have created exciting opportunities to study inherited arrhythmias. iPSC-CMs have been generated from patients with CPVT as well as other inherited arrhythmias and have been shown to capture key features of these diseases, including abnormal action potential duration and drug responses. However, current studies have been limited to isolated cells or cell clusters, leaving a large gap to modeling clinical arrhythmias, which are the emergent properties of cells assembled into myocardial tissue.

### AIP and Analogs

Also included in the invention are adenoviral or adeno-associated viral vectors encoding AIP polypeptides or fragments thereof that are modified in ways that enhance or do not inhibit their ability to modulate cardiac rhythm. In one embodiment, the invention provides methods for optimizing an AIP amino acid sequence or nucleic acid sequence by producing an alteration. Such changes may include certain mutations, deletions, insertions, post-translational modifications, and tandem replication. In one preferred embodiment, the AIP amino acid sequence is modified to enhance protease resistance, particularly metalloprotease resistance. Accordingly, the invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 10, 13, 15 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta or gamma amino acids.

In addition to full-length polypeptides, the invention also includes fragments of any one of the polypeptides of the invention. As used herein, the term “a fragment” means at least 5, 6, 7, 8, 9, 10, 11, 12, or 13 amino acids in length. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein AIP analogs having a chemical structure designed to mimic AIP functional activity (e.g., cardiac regulatory activity) can be administered according to methods of the invention. AIP analogs may exceed the physiological activity of native AIP. Methods of analog design are well known in the art, and synthesis of analogs can be carried out according to such methods by modifying the chemical structures such that the resultant analogs exhibit the immunomodulatory activity of a native AIP. These chemical modifications include, but are not limited to, substituting alternative R groups and varying the degree of saturation at specific carbon atoms of the native AIP. Preferably, the AIP analogs are relatively resistant to in vivo degradation, resulting in a more prolonged therapeutic effect upon administration. Assays for measuring functional activity include, but are not limited to, those described in the Examples below.

This invention also contemplates methods to increase the specificity and potency of CaMKII inhibition to its action on RYR2. For instance, the inhibitory peptide may be localized to RYR2 by expression of a fusion protein containing and RYR2 binding module and a CaMKII inhibitor sequence. The RYR2 binding module might consist of FKBP12.6 or a derivative of FKBP12.6.

### Polynucleotide Therapy

Polynucleotide therapy featuring a polynucleotide encoding an AIP peptide, analog, variant, or fragment thereof is another therapeutic approach for treating a cardiac arrhythmia (e.g., CPVT). Expression of such proteins in a cardiac cell is expected to modulate function of the cardiac cell, tissue, or organ, for example, by inhibiting phosphorylation of RYR2, inhibiting CAMKII activity, or otherwise regulating cardiac rhythm. Such nucleic acid molecules can be delivered to cells of a subject having a cardiac arrhythmia. The nucleic acid molecules must be delivered to the cells of a subject in a form in which they can be taken up so that therapeutically effective levels of an AIP peptide or fragment thereof can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an AIP peptide, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In one embodiment, a viral vector is used to administer a polynucleotide encoding an AIP peptide to a cardiac tissue.

Transducing viral vectors have tissue tropisms that permit selective transduction of one cell type compared to another. For instance, while CAMKII inhibition in cardiomyocytes will be therapeutic for CPVT or other forms of heart disease, its inhibition in other tissues, such as the brain, may not be desirable. In some embodiments, vectors that target cardiomyocytes with high specificity compared to other cell types are used. This would allow specific cardiac targeting of the expression of the CAMKII inhibitor peptide molecule. This is because CAMKII inhibition in other non-cardiac cell can be deleterious. Among potential adeno-associated virus candidates are AAV9, AAV6, AAV2i8, Anc80, and Anc82. Adeno-associated virus transduction efficiency is enhanced when the genome is “self-complimentary.” In some embodiments, self-complementary adeno-associated virus is used to increase the cardiac transduction by the gene therapy vector.

Non-viral approaches can also be employed for the introduction of therapeutic to a cardiac cell of a patient requiring inhibition of CPVT. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), the CMV-chicken b-actin hybrid promoter (“CAG”), or metallothionein promoters, and regulated by any appropriate mammalian regulatory element. For treatment of CPVT, it is desirable to selectively express the CAMKII inhibitor in cardiomyocytes and to minimize expression in other cell types. In some embodiments, cardiomyocyte-selective promoters are used for the expression of the CAMKII inhibitor peptide. The promoters or enhancers used can include, without limitation, those that are characterized as tissue- or cell-specific enhancers. For example, the cardiac troponin T promoter, the a-myosin heavy chain (a-MHC) promoter, the myosin light chain-2v (MLC-2v) promoter or the cardiac NCX1 promoter can be used to direct expression in cardiomyocytes. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic CaMKII inhibitor, such as a recombinant AIP peptide, variant, or fragment thereof, either directly to the site of a potential or actual disease-affected tissue or systemically (for example, by any conventional recombinant protein administration technique). The dosage of the administered peptide depends on a number of factors, including the size and health of the individual patient. For any particular subject, the specific dosage regimes should be adjusted over time according to the individual need and the professional judgment of the person administering or supervising the administration of the compositions.

### Screening Assays

The invention provides methods for modifying a cardiac rhythm by administering a CAMKII inhibitor, AIP or an analog thereof, or a polynucleotide encoding AIP. While the Examples described herein specifically discuss the use of an AAV vector encoding an AIP peptide, one skilled in the art understands that the methods of the invention are not so limited. Virtually any agent that inhibits the phosphorylation of a cardiac ryanodine channel (RYR2) by CAMKII may be employed in the methods of the invention. Exemplary CAMKII inhibitors are known in the art and described herein.

Methods of the invention are useful for the high-throughput low-cost screening of candidate agents that inhibit CPVT or that advantageously regulate a cardiac rhythm. Such agents can be identified using, for example, human iPSC-derived cardiomyocytes that express optogenetic actuators or sensors. A candidate agent that specifically inhibits CPVT, inhibits CaMKII phosphorylation of RYR2 is then isolated and tested for activity in an in vitro assay or in vivo assay for its ability to inhibit CPVT, desirably modulate a cardiac rhythm or other cardiac function. One skilled in the art appreciates that the effects of a candidate agent on a cell, tissue or organ is typically compared to a corresponding control cell, tissue or organ not contacted with the candidate agent. Thus, the screening methods include comparing the properties of the contacted cell to the properties of an untreated control cell.

Agents that mimic the effects of AIP, i.e., agents that inhibit CPVT, inhibit phosphorylation of RYR2 by CaMKII or otherwise regulate a cardiac rhythm may be used, for example, as therapeutics to regulate a cardiac rhythm. Each of the polynucleotide sequences provided herein may also be used in the discovery and development of such therapeutic compounds. The encoded AIP peptides and analogs thereof, upon expression, can be used to prevent CPVT in a subject.

### Test Compounds and Extracts

In general, CaMKII inhibitors, AIP peptide analogs and mimetics are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of cardiac arrhythmias. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Patent No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have cardiac rhythm regulatory activity or CAMKII inhibitory activity further fractionation of the positive lead extract is necessary to isolate molecular constituents responsible for the observed effect. Thus, the goal of the extraction, fractionation, and purification process is the careful characterization and identification of a chemical entity within the crude extract having the desired activity. Methods of fractionation and purification of such heterogenous extracts are known in the art. If desired, compounds shown to be useful as therapeutics are chemically modified according to methods known in the art.

### Therapeutic Methods

Agents identified as a CaMKII inhibitor, having AIP mimetic activity (e.g., CaMKII inhibitory activity, cardiac rhythm regulatory activity) and/or polynucleotides encoding an AIP or AIP analog are useful for preventing or ameliorating CPVT or another cardiac arrhythmia. Diseases and disorders characterized by cardiac arrhythmia may be treated using the methods and compositions of the invention.

In one therapeutic approach, an agent identified as described herein is administered to the site of a potential or actual disease-affected tissue or is administered systemically. The dosage of the administered agent depends on a number of factors, including the size and health of the individual patient. For any particular subject, the specific dosage regimes should be adjusted over time according to the individual need and the professional judgement of the person administering or supervising the administration of the compositions.

### Pharmaceutical Therapeutics

The invention provides a simple means for identifying compositions (including polynucleotides, peptides, small molecule inhibitors, and AIP mimetics) having CaMKII inhibitory activity and/or cardiac rhythm regulatory activity. Accordingly, a chemical entity discovered to have medicinal value using the methods described herein is useful as a drug or as information for structural modification of existing compounds, e.g., by rational drug design. Such methods are useful for screening agents having an effect on a variety of conditions characterized by a cardiac arrhythmia.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. For AAV gene therapy, administration may be intravenous or intracoronary. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the cardiac arrhythmia. Generally, amounts will be in the range of those used for other agents used in the treatment of other diseases requiring regulation of cardiac function, although in certain instances lower amounts will be needed because of the increased specificity of the compound. A compound is administered at a dosage having CAMKII inhibitory activity or cardiac rhythm regulatory activity as determined by a method known to one skilled in the art, or using any that assay that measures the expression or the biological activity of a CAMKII polypeptide.

### Formulation of Pharmaceutical Compositions

The administration of a compound for the treatment of cardiac arrhythmia may be by any suitable means that results in a concentration of the therapeutic that, combined with other components, is effective in ameliorating, reducing, or stabilizing a cardiac arrhythmia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, N.Y.).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a cardiac arrhythmia by using carriers or chemical derivatives to deliver the therapeutic agent to a particular cell type (e.g., cardiac cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, intracoronary or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a cardiac arrhythmia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active a cardiac active therapeutic substance). The coating may be applied on the solid dosage form in a similar manner as that described in Encyclopedia of Pharmaceutical Technology, supra.

In one embodiment, two or more cardiac therapeutics may be mixed together in the tablet, or may be partitioned. In one example, the first active cardiac therapeutic is contained on the inside of the tablet, and the second active therapeutic is on the outside, such that a substantial portion of the second therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active cardiac therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, a cardiac therapeutic described herein (e.g., CAMKII inhibitor, AIP peptide or polynucleotide) may be administered in combination with any other standard therapy useful for regulating cardiac function; such methods are known to the skilled artisan and described in Remington's Pharmaceutical Sciences by E. W. Martin.

### Genome Editing of Mutant RYR2

Because subjects comprising a RYR2 mutation are predisposed to CPVT, it would be desirable to specifically repair the defective gene encoding the RYR2 polypeptide. Therapeutic gene editing is a major focus of biomedical research, embracing the interface between basic and clinical science. A large number of different recessive hereditary human disease syndromes are caused by inheritance of biallelic inactivating point mutations of disease genes. The development of novel “gene editing” tools provides the ability to manipulate the DNA sequence of a cell at a specific chromosomal locus, without introducing mutations at other sites of the genome. This technology effectively enables the researcher to manipulate the genome of a subject's cells in vitro or in vivo, to effect a reversion of a deleterious genotype (e.g., the gene encoding RYR2^{R4651I}). Altneratively, since the inventors have discovered that phosphorylation of RYR2-S2814 by CAMKII unmasks CPVT mutations, and that the RYR2-S2814A mutation is protective, therapeutic gene editing may involve introduction of an S2814A mutation into patient cardiomyocytes to make them less vulnerable to arrhythmia.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule is introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. Importantly, if the donor DNA molecule differs slightly in sequence from the chromosomal sequence, HR-mediated DSB repair will introduce the donor sequence into the chromosome, resulting in gene conversion/gene correction of the chromosomal locus. In the context of therapeutic gene targeting, the altered sequence chosen would be an active or functional fragment (e.g., wild type, normal) of the disease gene of interest. By targeting the nuclease to a genomic site that contains the disease-causing point mutation, the concept is to use DSB formation to stimulate HR and to thereby replace the mutant disease sequence with wild-type sequence (gene correction). The advantage of the HR pathway is that it has the potential to generate seamlessly a wild type copy of the gene in place of the previous mutant allele.

Current genome editing tools use the induction of double strand breaks (DSBs) to enhance gene manipulation of cells. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for ˜18 bp sequences in the genome.

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15;339(6121):823-6). Unlike other gene therapy methods, which add a functional, or partially functional, copy of a gene to a patient's cells but retain the original dysfunctional copy of the gene, this system can remove the defect. Genetic correction using engineered nucleases has been demonstrated in tissue culture cells and rodent models of rare diseases.

CRISPR has been used in a wide range of organisms including bakers yeast (Saccharomyces cerevisiae), zebra fish, nematodes (Caenorhabditis elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

See also U.S. Patent Publication 2014/0068797, which is incorporated by reference in its entirety.

Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17;337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

In one approach, one or more cells of a subject are altered to express a wild-type form of RYR2R^{4651I} using a CRISPR-Cas system. Cas9 can be used to target a RYR2^{R4651I} comprising a mutation. Upon target recognition, Cas9 induces double strand breaks in the RYR2^{R4651I} target gene. Homology-directed repair (HDR) at the double-strand break site can allow insertion of a desired wild-type RYR2^{R4651I} sequence.

The following US patents and patent publications are incorporated herein by reference: U.S. Pa. No. 8,697,359, 20140170753, 20140179006, 20140179770, 20140186843, 20140186958, 20140189896, 20140227787, 20140242664, 20140248702, 20140256046, 20140273230, 20140273233, 20140273234, 20140295556, 20140295557, 20140310830, 20140356956, 20140356959, 20140357530, 20150020223, 20150031132, 20150031133, 20150031134, 20150044191, 20150044192, 20150045546, 20150050699, 20150056705, 20150071898, 20150071899, 20150071903, 20150079681, 20150159172, 20150165054, 20150166980, and 20150184139.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Isogenic CPVT (PGP1-RYR2^{R4651I})

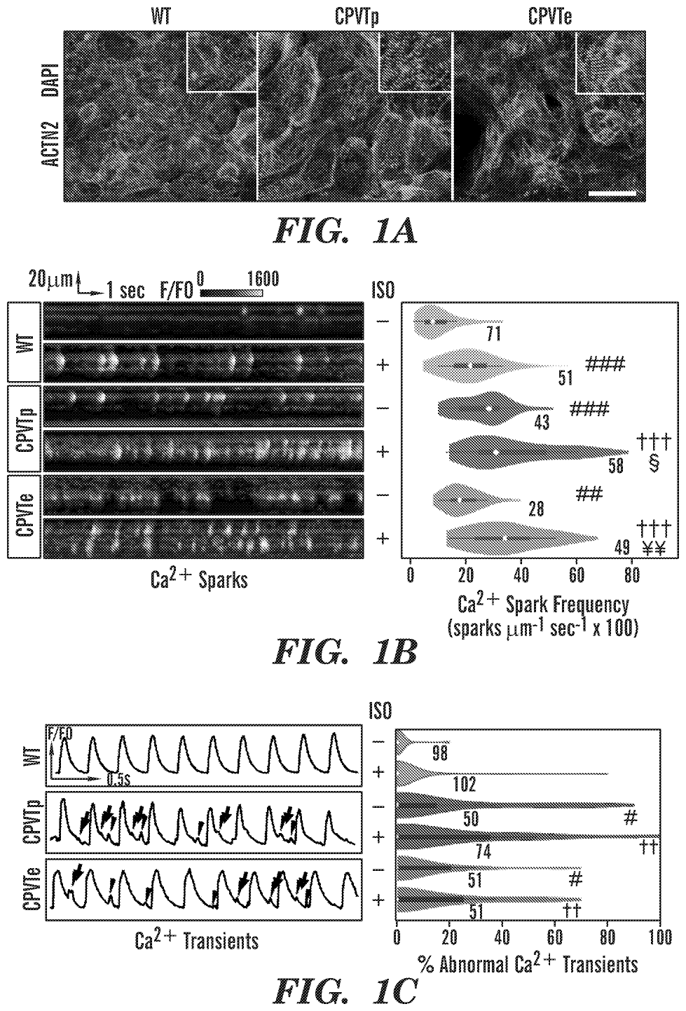

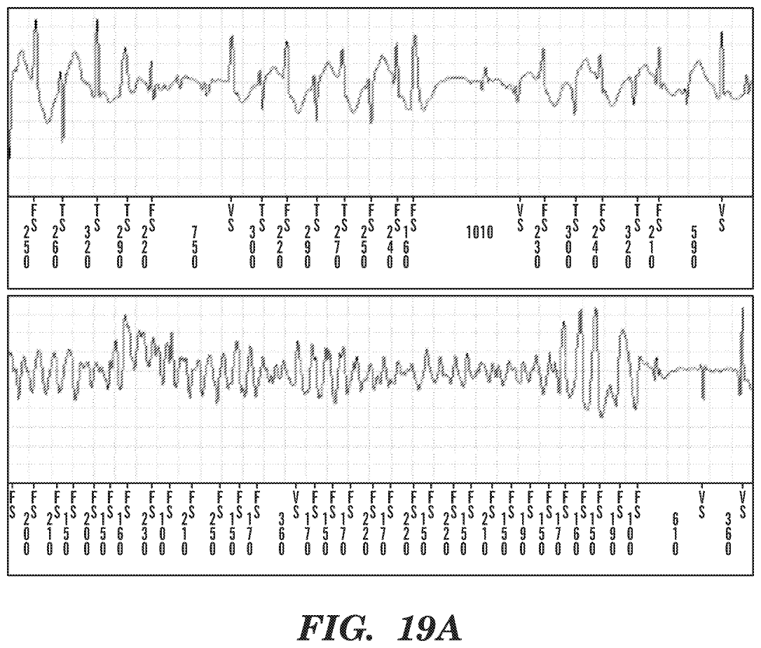

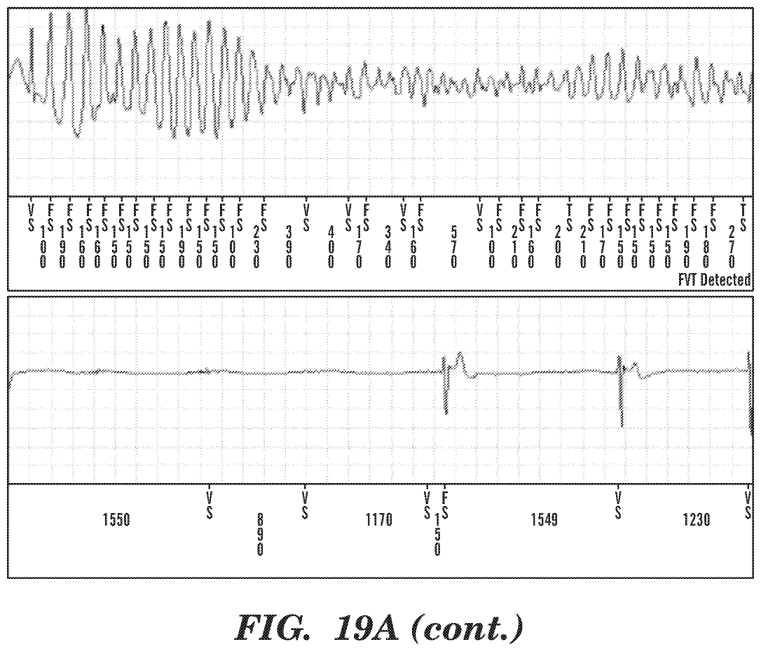

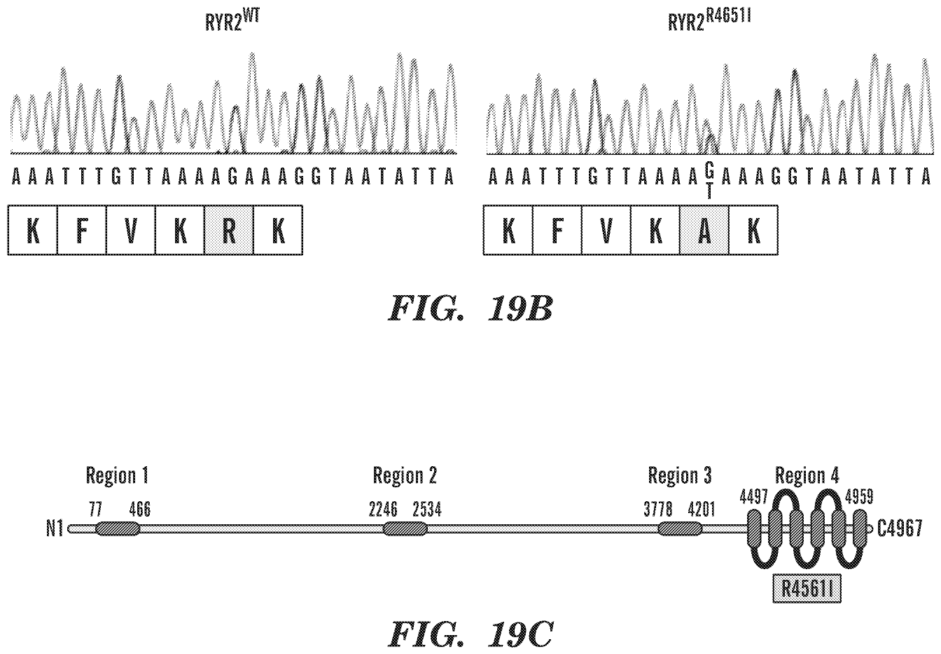

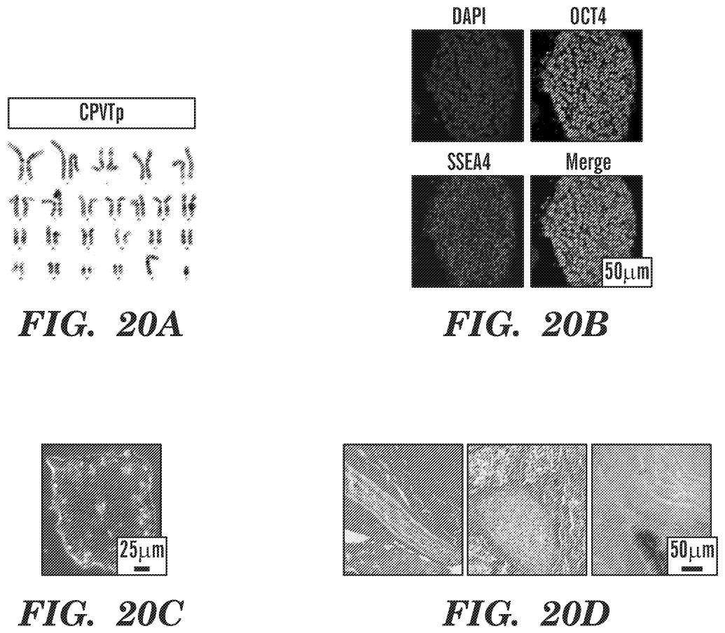

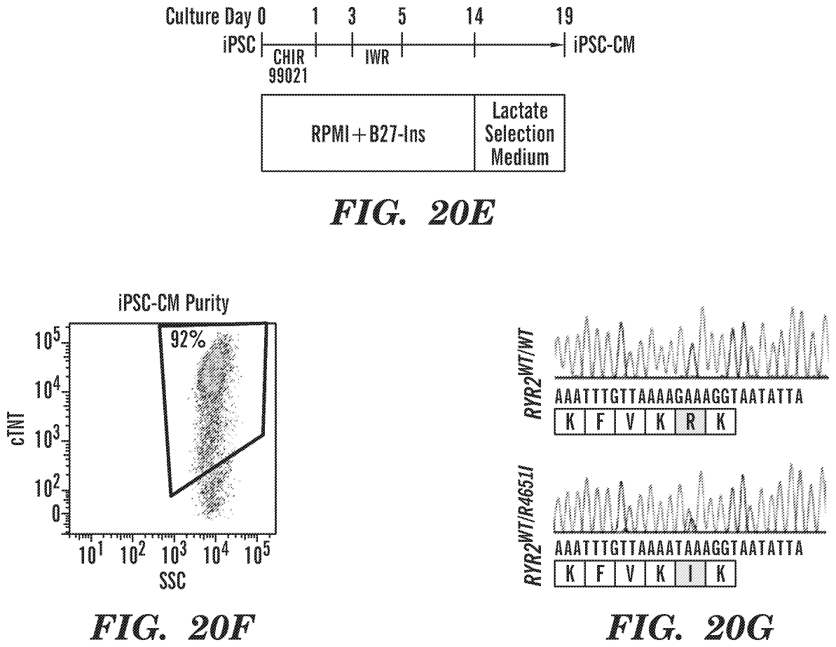

Skin fibroblasts were obtained from a CPVT patient. This patient had a normal resting electrocardiogram, but exercise-induced polymorphic ventricular tachycardia. Genotyping revealed that the patient had a point mutation in RYR2 that caused substitution of isoleucine for arginine at position 4651 (R4651I; FIGS. 19A-19C). Clinical genotyping did not implicate other candidate inherited arrhythmia genes. The fibroblasts were reprogrammed into iPSCs (line CPVTp, where p indicates patient-derived; FIGS. 20A-20D), which robustly differentiated into iPSC-CMs with comparable efficiency to the wild-type iPSC line PGP1 (FIGS. 1A, 20E-20F). Cas9 genome editing was used to introduce the patient mutation into PGP1, yielding isogenic CPVT (PGP1-RYR2^{R4651I}, abbreviated CPVTe, where e denotes engineered) and control (PGP1, abbreviated WT) lines (FIG. 20G).

Ca²⁺ handling of wild-type (WT), CPVTp, and CPVTe iPSC-CMs was analyzed by loading spontaneously beating, isolated cell islands with the Ca²⁺ -sensitive dye Fluo-4 and confocal line scan imaging. Compared to WT, both patient-derived (CPVTp) and genome-edited isogenic iPSC-CMs (CPVTe) had more frequent spontaneous Ca²⁺ release events at individual Ca²⁺ release units, known as Ca²⁺ sparks, and this was further exacerbated by isoproterenol (ISO), a beta-sympathomimetic (FIG. 1B). RYR2 function was examined by recording calcium transients. At baseline, CPVTp and CPVTe had dramatically increased after-depolarization frequency (FIG. 1C). With isoproterenol stimulation, after-depolarization frequency remained markedly elevated in CPVTp and CPVTe compared to WT.

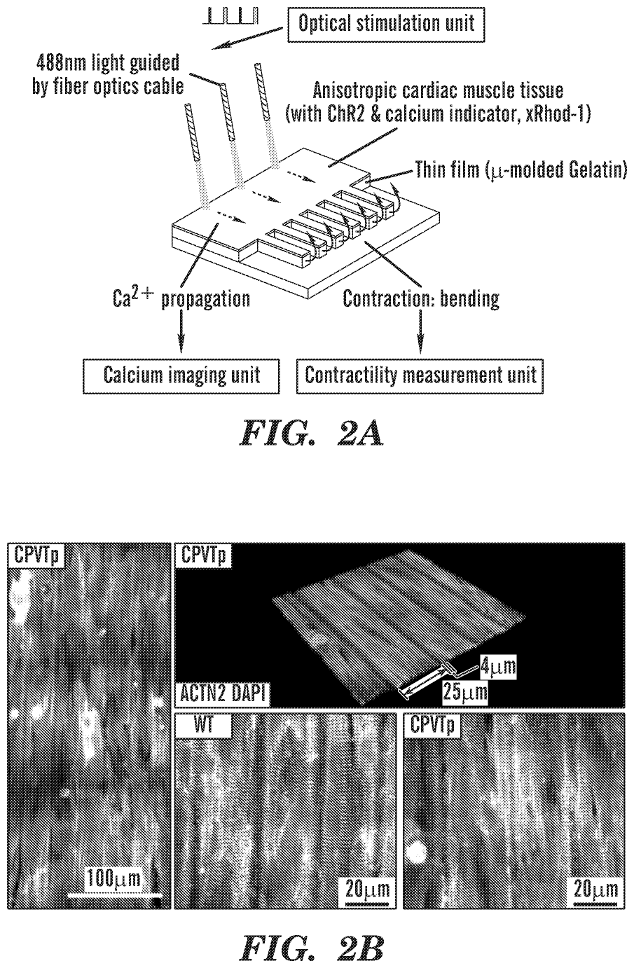

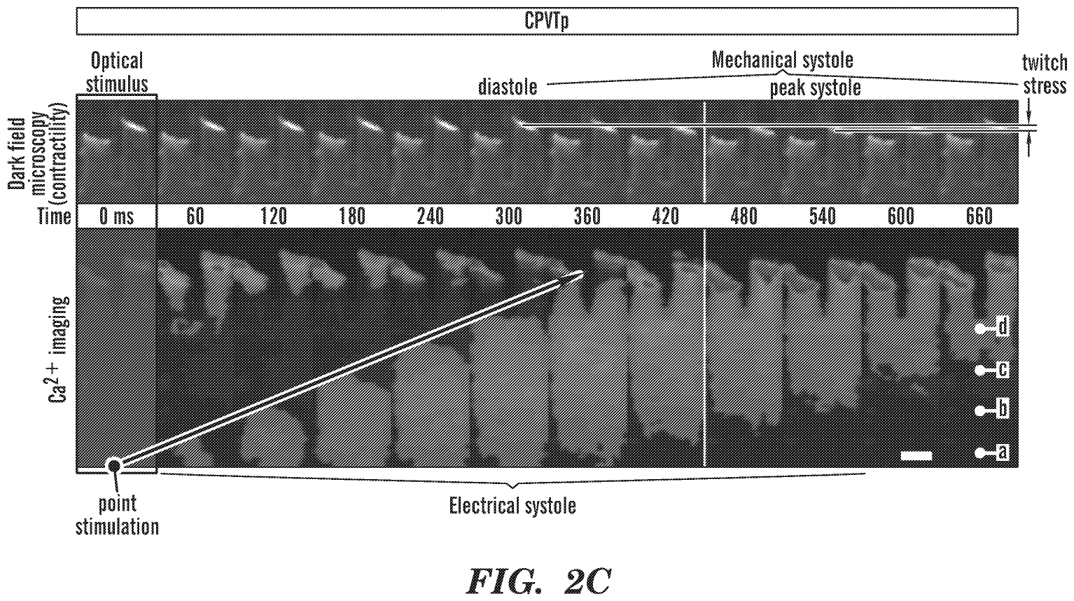

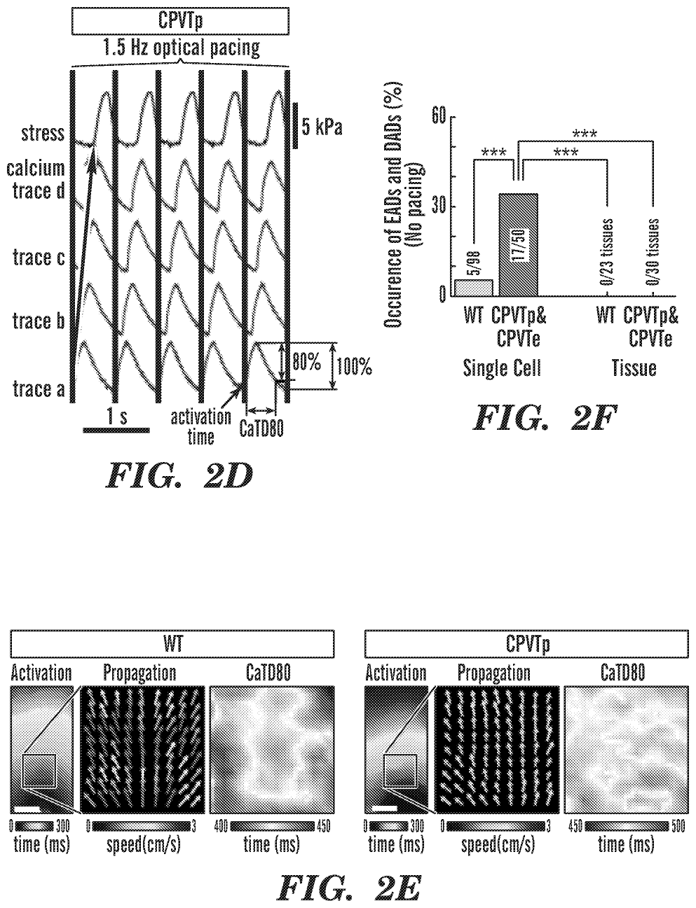

Since clinical arrhythmias emerge from the collective behavior of cardiomyocytes assembled into tissues, to better model inherited arrhythmias, muscular thin films were integrated (MTF), optogenetics, and optical mapping to yield “opto-MTFs”, a platform that permits simultaneous assessment of myocardial conduction and contraction (FIG. 2A).

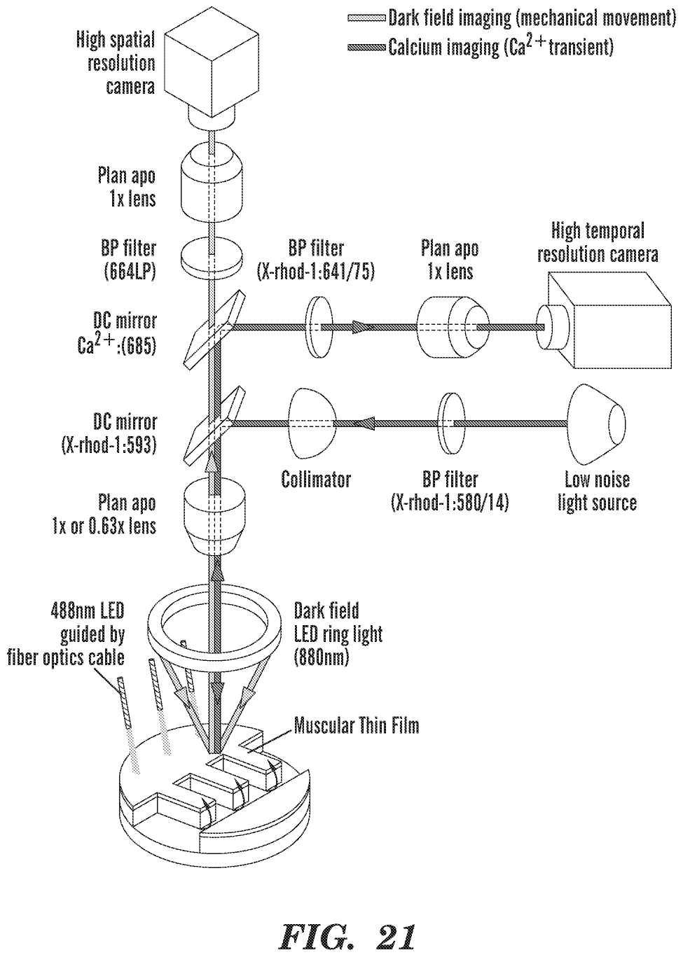

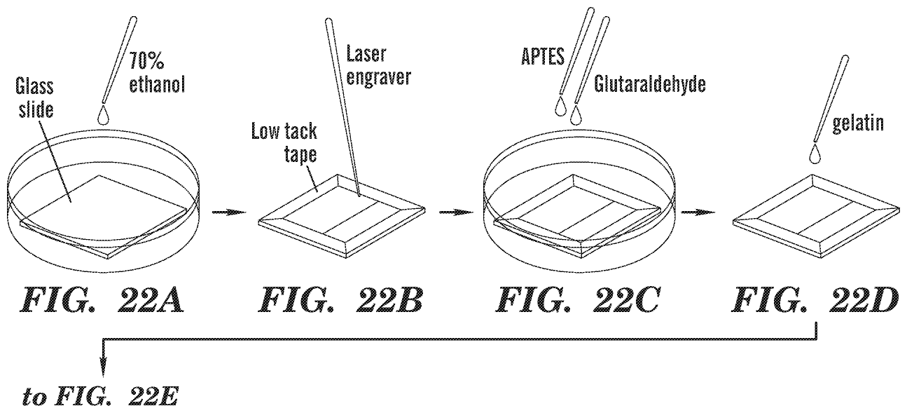

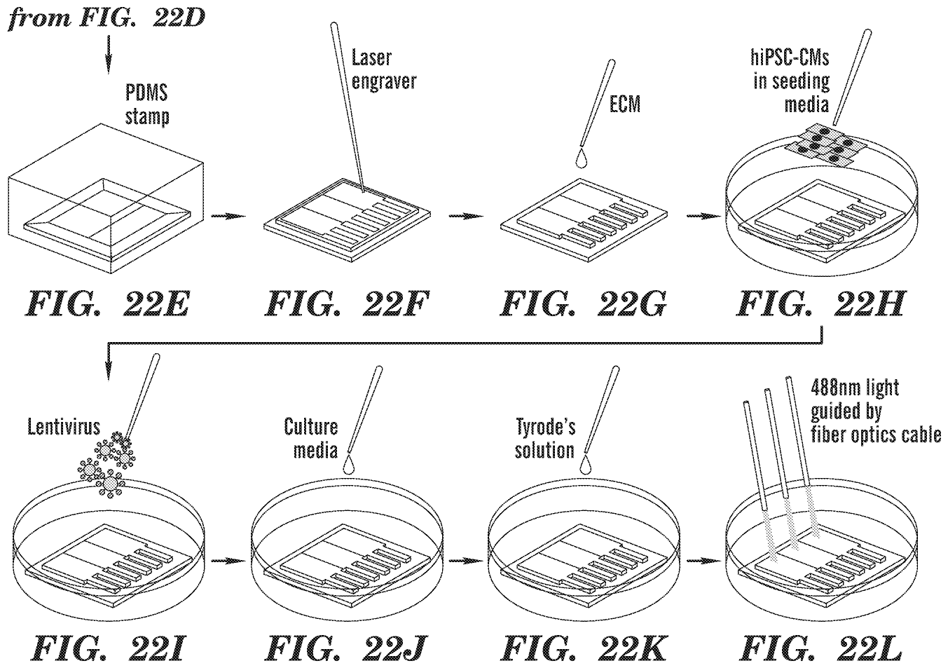

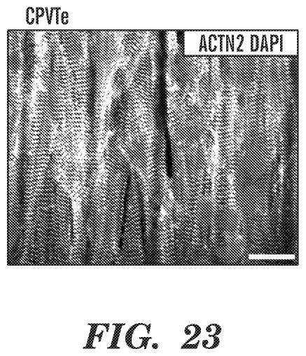

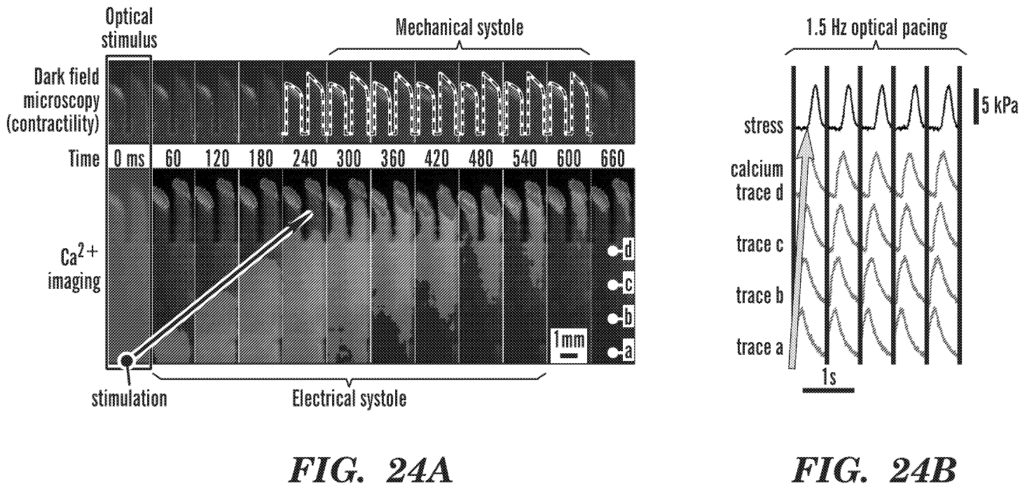

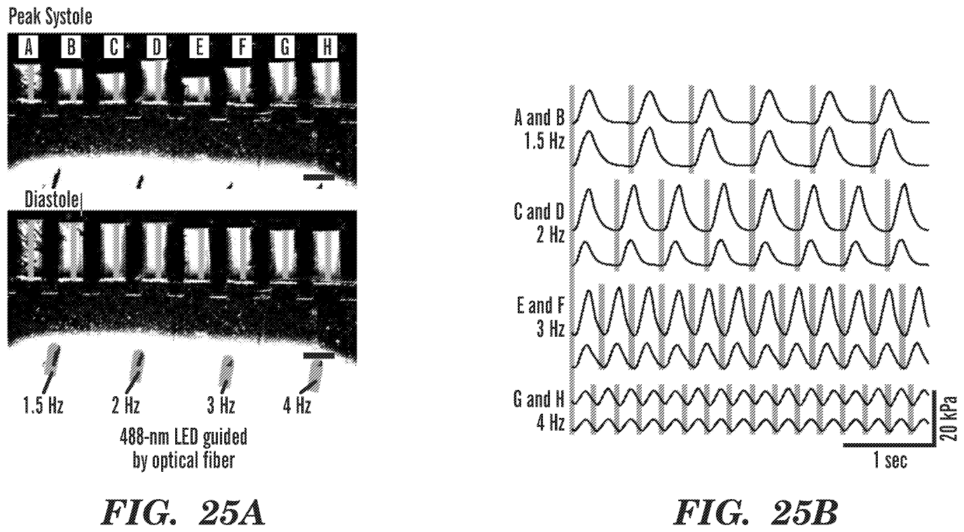

Lentivirus was used to program cardiomyocytes to express channel rhodopsin (ChR2), a light-gated channel, as described. In pilot experiments, ChR2 expression in cardiomyocytes enabled optical pacing using blue light without measurably affecting their electrical activity (FIG. 21). Light-responsive, ChR2-expressing cardiomyocytes were seeded on micro-molded gelatin chips (FIG. 22H), so that they assembled with the parallel alignment characteristic of native myocardium (FIGS. 2A-2B). Blue LED light illumination (470 nm, 10 msec pulses) directed through an optical fiber illuminated a ˜0.79 mm² region, containing ˜500 cells, at one end of the 3×10 mm MTFs. This was sufficient to elicit action potentials waves that conducted across the MTFs along the long axis of muscle fibers (FIG. 2A). Upon reaching two film cantilevers located at the other end of the MTF, action potential waves induced iPSC-CM contraction, displacing the cantilever (FIGS. 2A and 2C). Using fluorescent optical mapping with the Ca²⁺-sensitive dye X-Rhod-1 in combination with dark field microscopy, calcium transients and contraction were simultaneously recorded. Calcium wave propagation followed by deflection of the cantilevers was clearly observed for MTFs assembled using control iPSC-CMs, demonstrating effective excitation-contraction coupling (FIGS. 2C and 24). Spatiotemporal characteristics of the MTFs, such as activation mapping, calcium transient duration, and conduction velocity were measured from the optical mapping data (FIG. 2D). Adjacent MTFs were independent of each other, and the optical stimulation system permitted each MTF to be separately controlled at different frequencies.

Having established the opto-MTF platform, it was used to characterize the tissue-scale properties of CPVT engineered heart tissues. The Ca²⁺ transient duration and conduction velocity of CPVT opto-MTFs did not differ significantly from controls (FIGS. 2E-2G and 24A). Whereas CPVT iPSC-CMs exhibited frequent after-depolarizations even at baseline (FIG. 1C), assembly into opto-MTF tissue largely abolished this aberrant activity (FIG. 2F). The CPVT tissue sheets thus better recapitulated the baseline phenotype of patients, who have few arrhythmias in the absence of exercise or emotional stress.

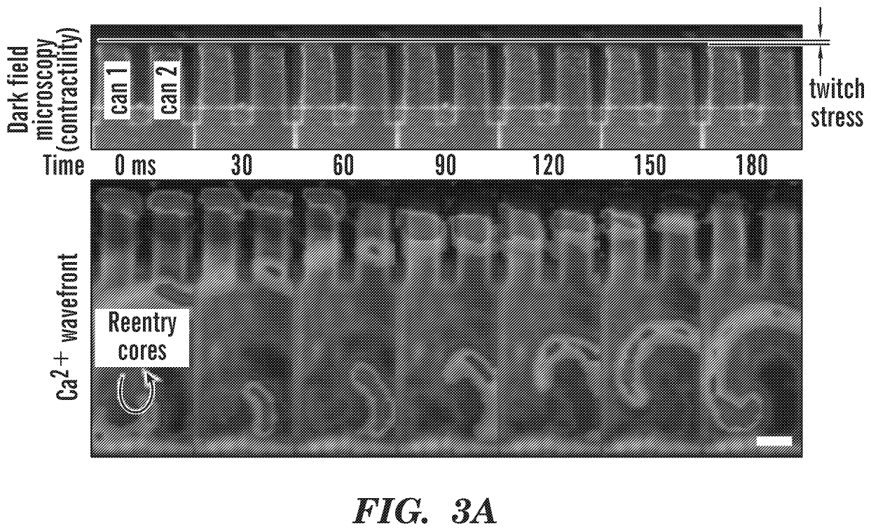

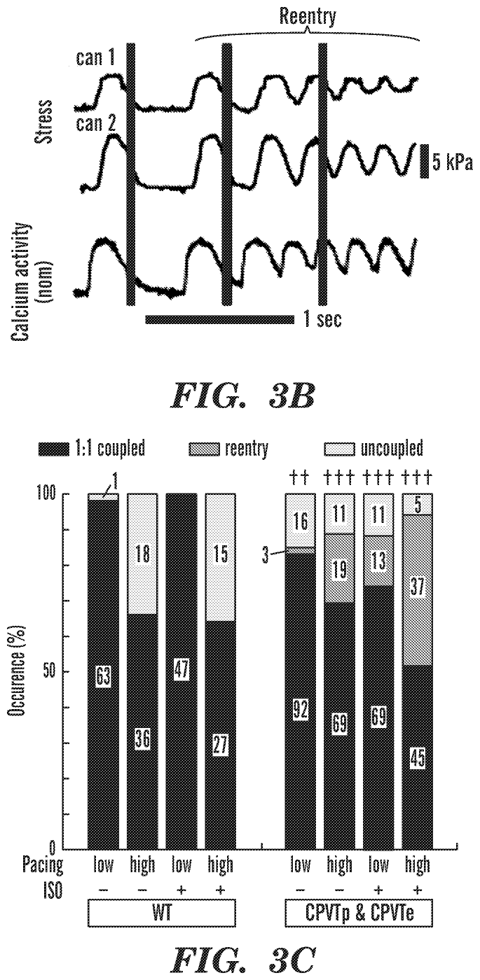

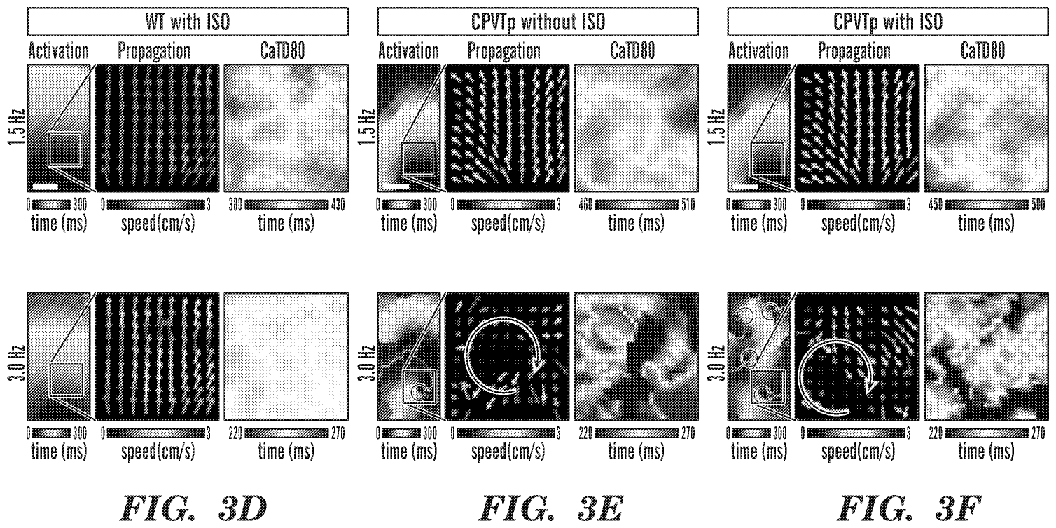

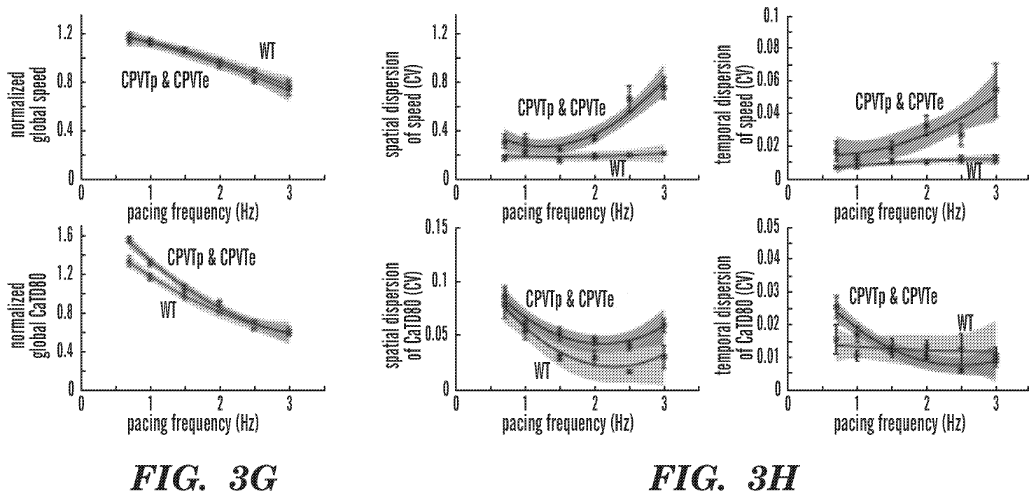

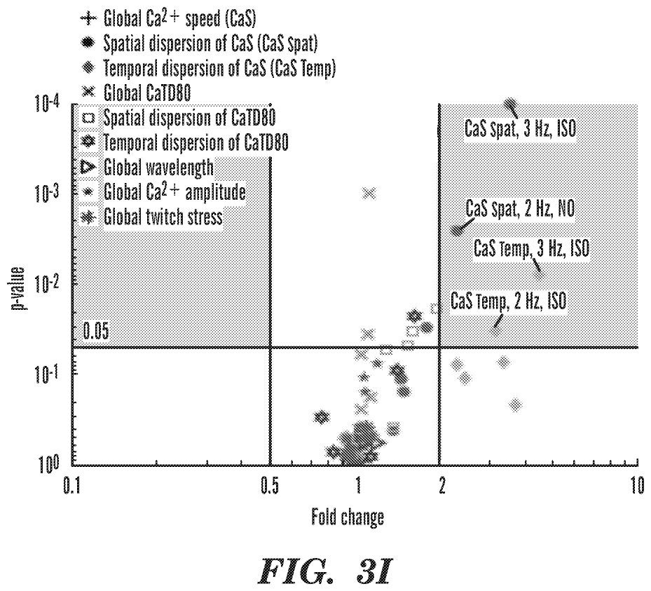

Patients with CPVT develop ventricular tachycardia during exercise or emotional stress. To simulate key features of these provocative conditions in vitro, opto-MTFs were stimulated with increasing optical pacing frequency (1-3 Hz) or β-adrenergic stimulation (0-10 μM ISO). Remarkably, treatment of CPVT, but not control opto-MTFs with either pacing or ISO induced a subset to sustain spiral wave re-entry, the tissue-level equivalent of ventricular tachycardia (FIGS. 3A-3C). The combination of both high pacing rate and ISO most potently evoked re-entry. During re-entry, the paired opto-MTF cantilevers moved asynchronously (FIG. 3D), mimicking the uncoordinated cardiac contraction that impairs cardiac output in clinical ventricular tachycardia. These data show that assembly of CPVT iPSC-CMs into opto-MTF models key features of the disease at a tissue level. Furthermore, the data implicate re-entry as an arrhythmia mechanism in CPVT.

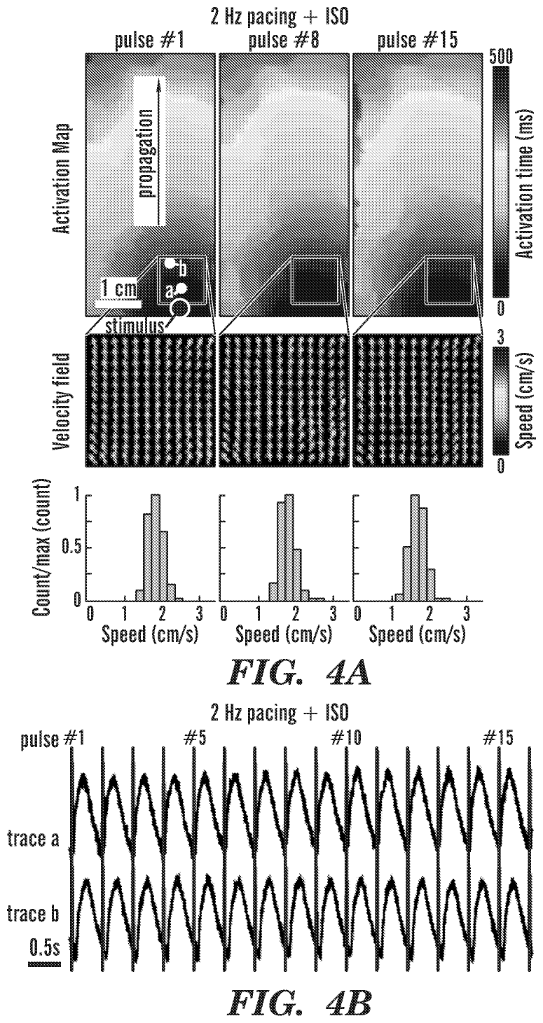

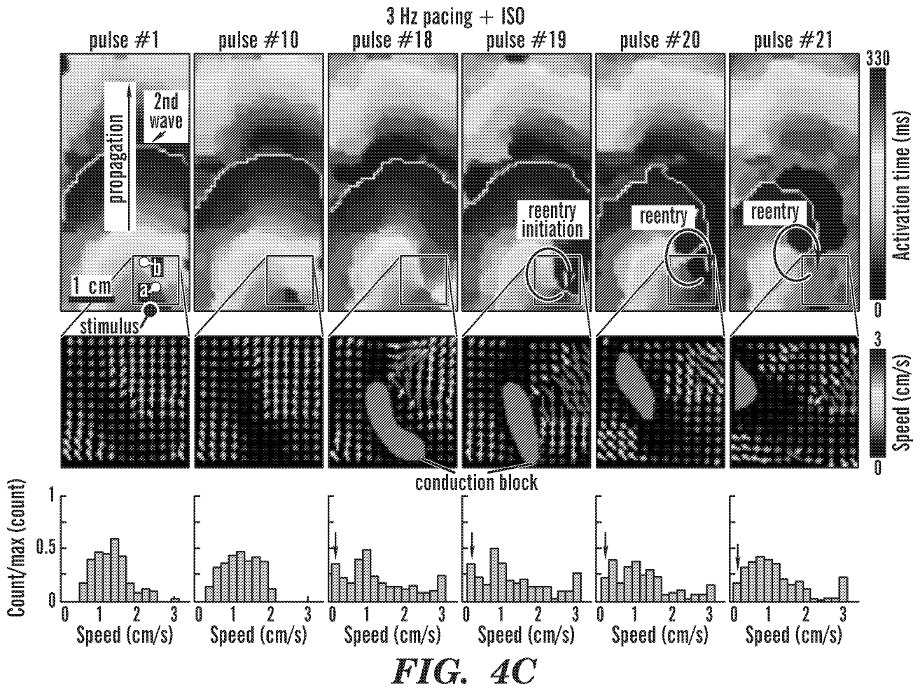

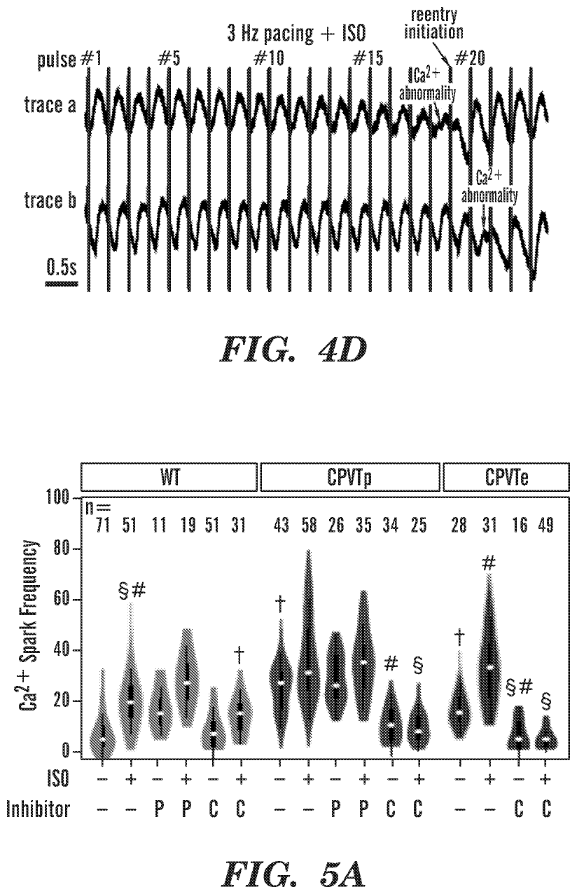

Heterogeneity of tissue excitability increases tissue vulnerability to re-entry. To investigate the cellular mechanisms that make CPVT tissues vulnerable to re-entry, the optical mapping data was analyzed to determine the effect of pacing rate and isoproterenol on dispersion of conduction velocity (CV) and Ca²⁺ transient duration (CaTD) across space and time. Only recordings with 1:1 capture and without re-entry were used for these analyses. With increasing pacing and ISO, CPVT tissues developed greater spatial and temporal dispersion of CV and CaTD than control tissues (FIG. 4A, 4B, and 24). These data suggest that RYR2 mutation increases heterogeneity of tissue excitability, creating a vulnerable substrate for development of re-entry.

Events that initiated re-entry in the vulnerable CPVT substrate were analyzed. Three recordings were identified that captured the initiation of re-entry (FIG. 4C). In each instance, an after-depolarization (FIG. 4C, arrow) occurred at the time of rotor initiation. The DAD was not sufficient to trigger, regional depolarization, but did cause regional conduction block and unidirectional impulse conduction, resulting in rotor formation. This role of sub-threshold DADs to initiate re-entry was predicted by a prior computational modeling study.

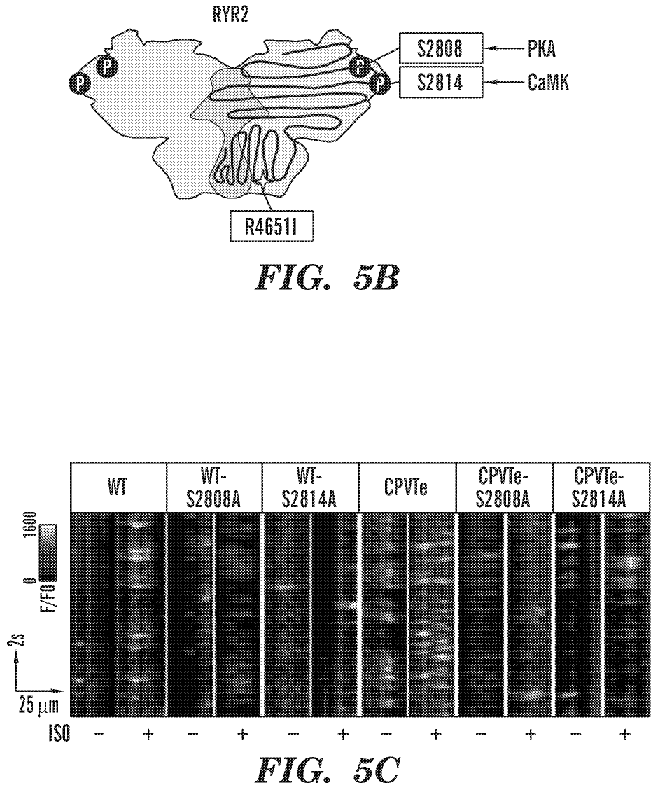

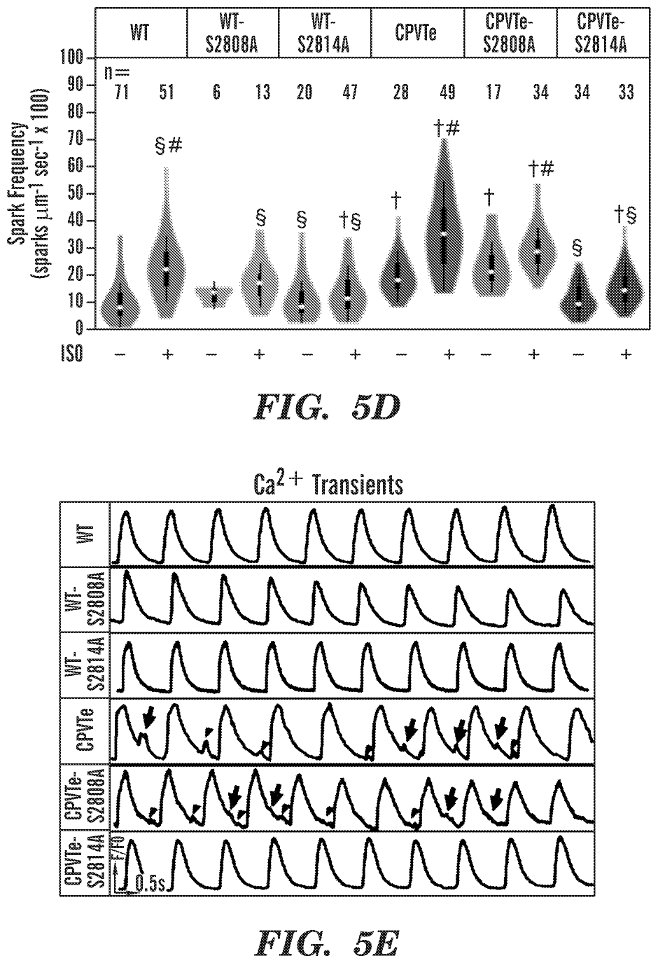

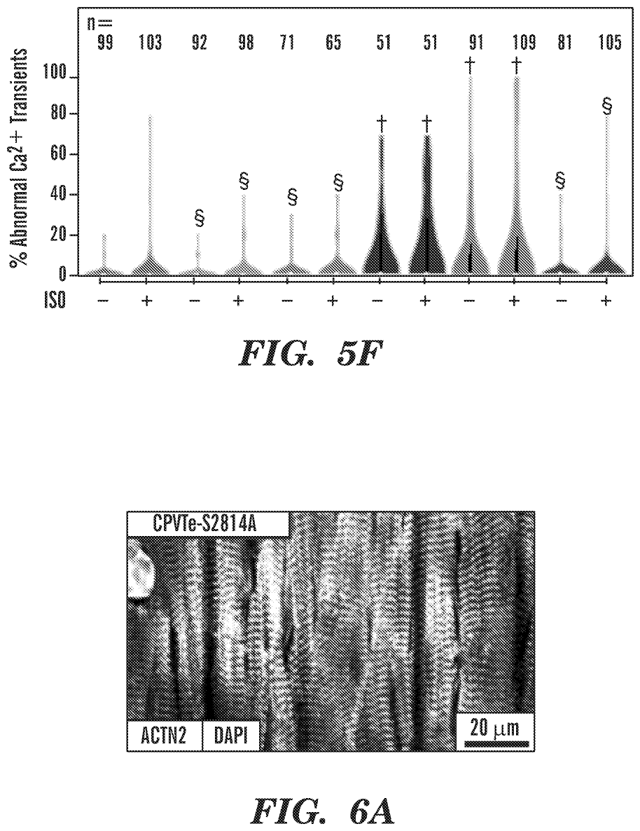

Although catecholamine stimulation is well known to provoke arrhythmia in CPVT, the molecular targets through which β-adrenergic stimulation unmasks the latent arrhythmic potential of RYR2 mutations were not known. β-adrenergic stimulation activates numerous signaling pathways, including Ca²⁺-calmodulin-dependent kinase II (CaMKII) and protein kinase A (PKA). Inhibition of PKA using a potent, cell-permeable peptide did not significantly reduce Ca²⁺ spark frequency in both patient-derived (CPVTp) and genetically engineered, isogenic (CPVTe) iPSC-CMs (FIG. 5A). In contrast, CaMKII inhibition with cell-permeable autocamtide inhibitory peptide (AIP), a highly selective and potent CaMKII inhibitor potently reduced Ca⁺ spark frequency in CPVT iPSC-CMs (FIGS. 5A and 25).

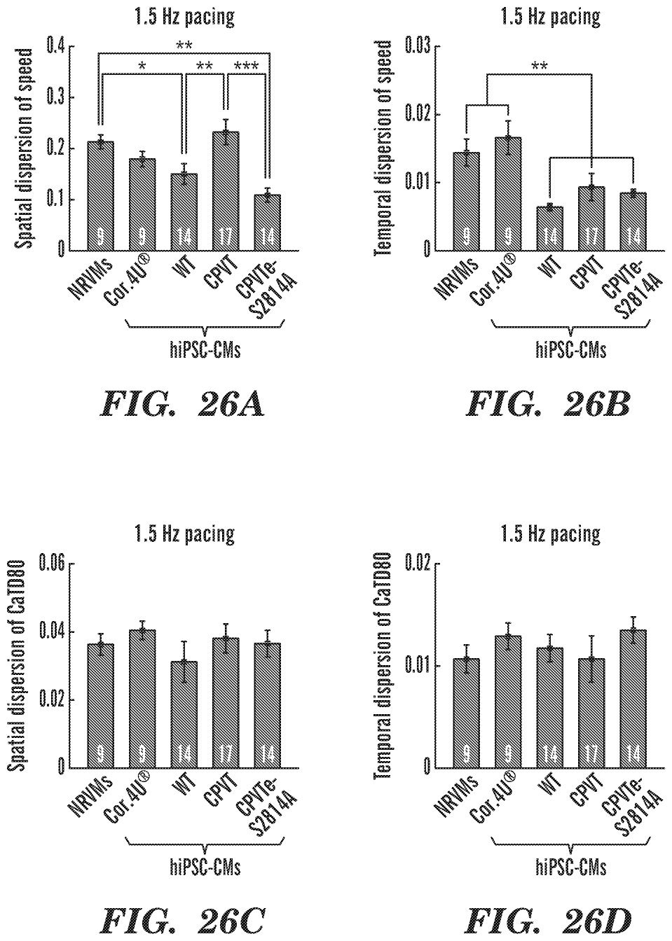

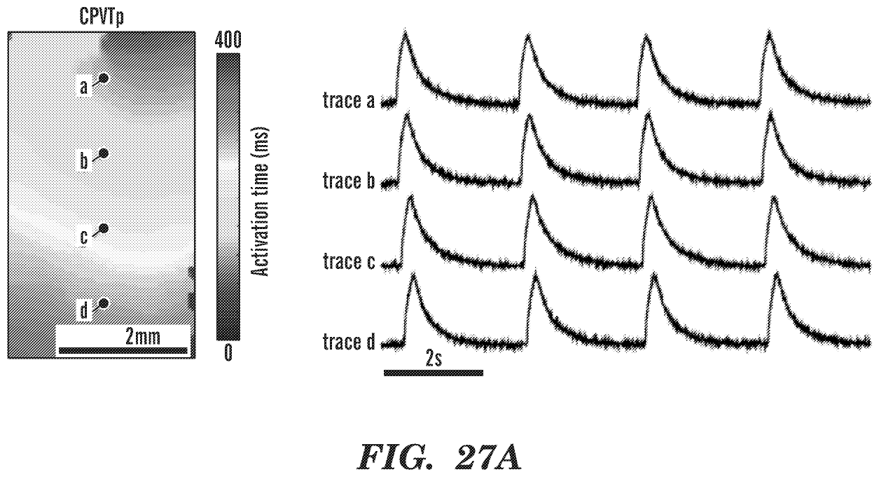

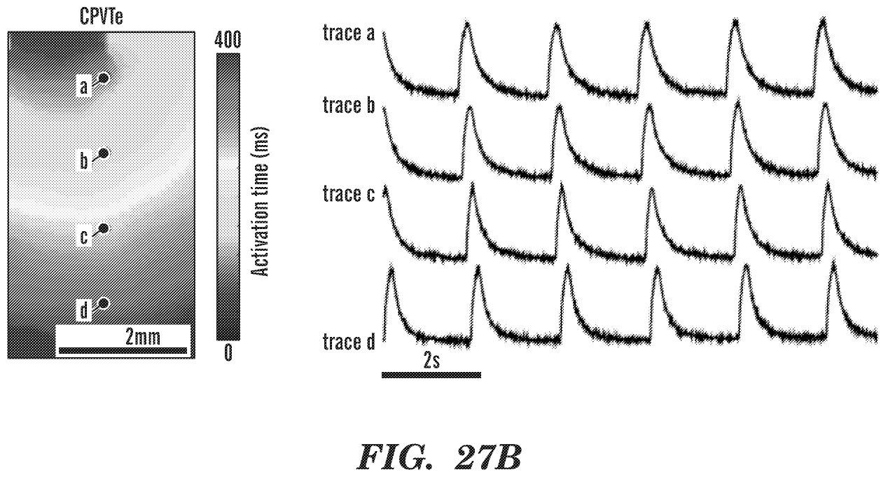

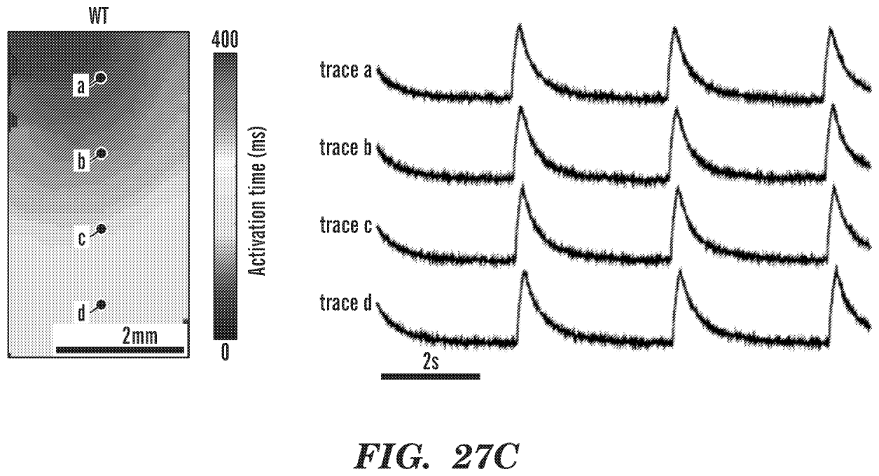

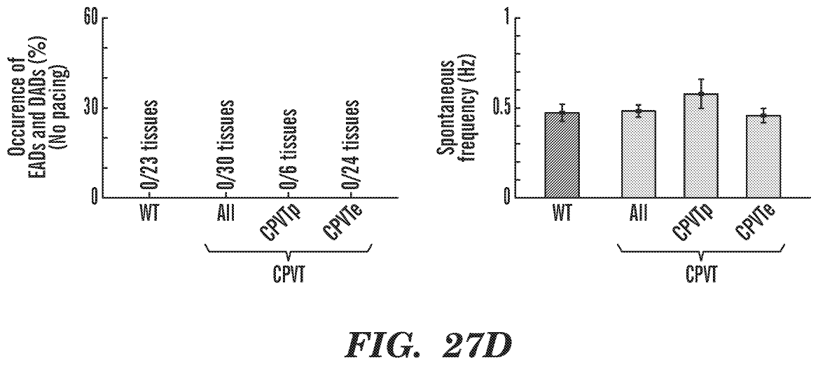

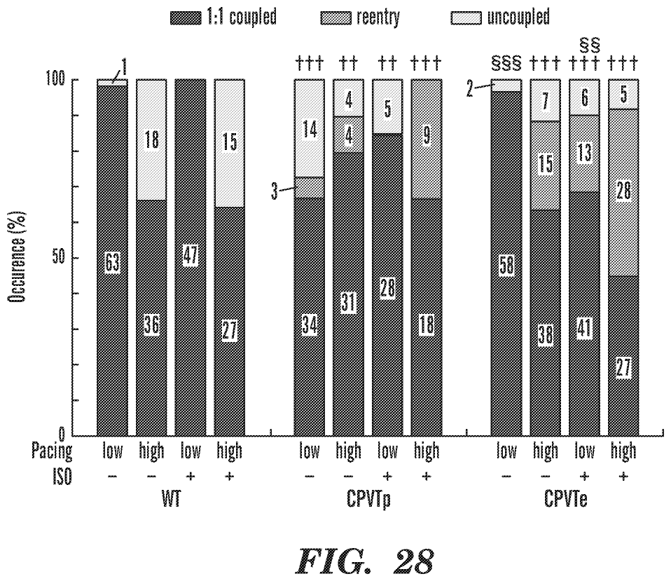

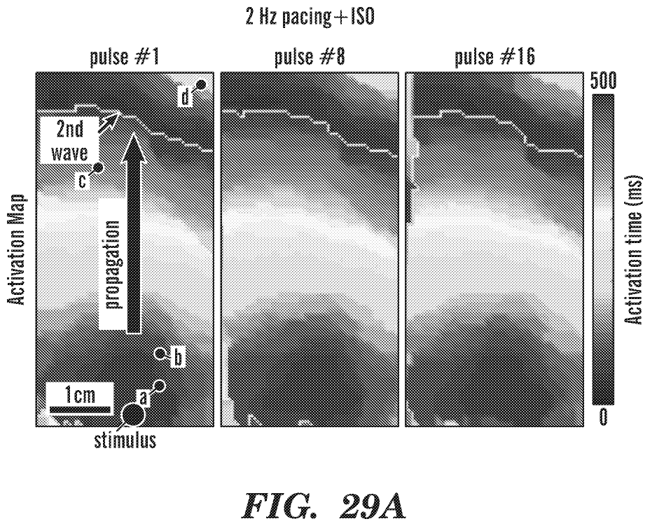

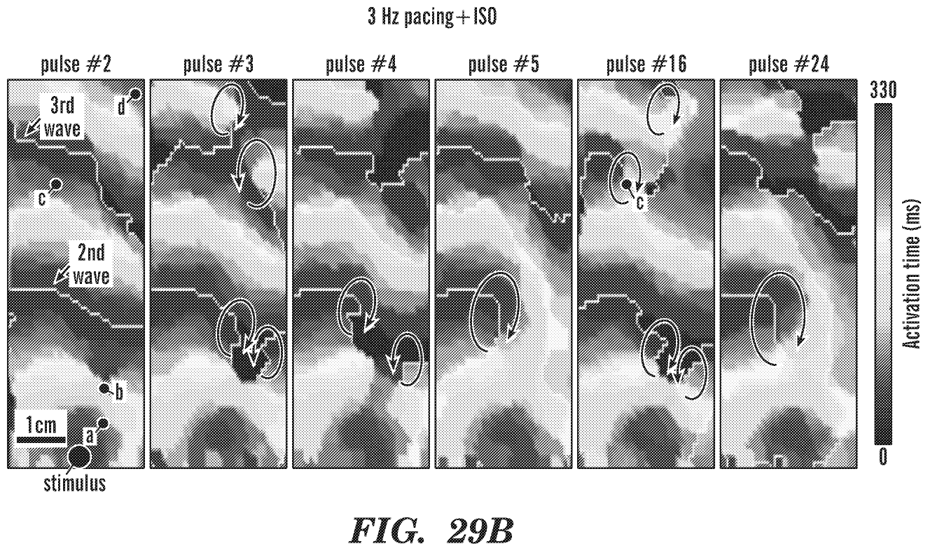

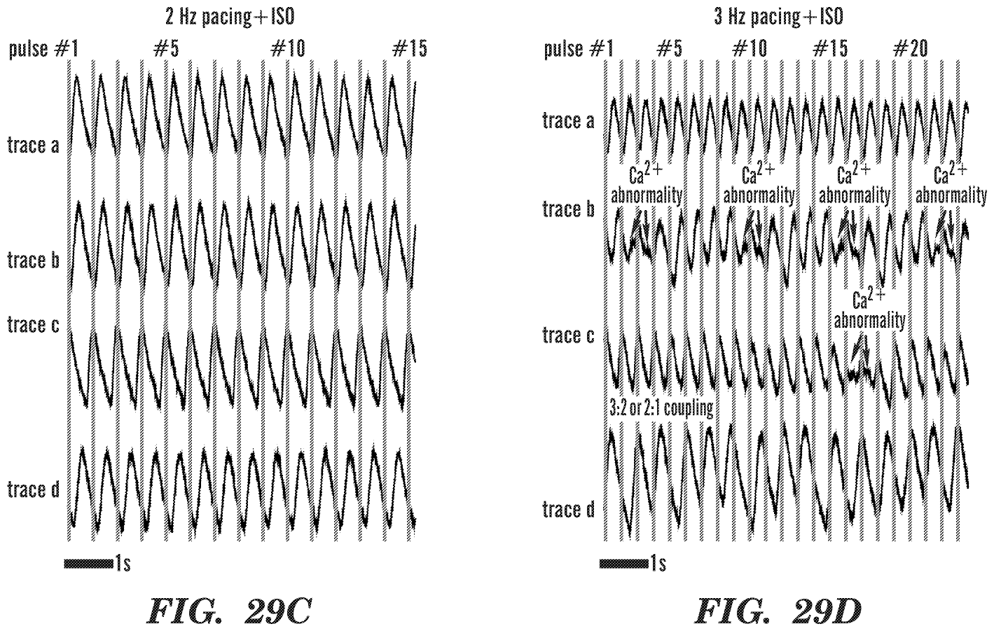

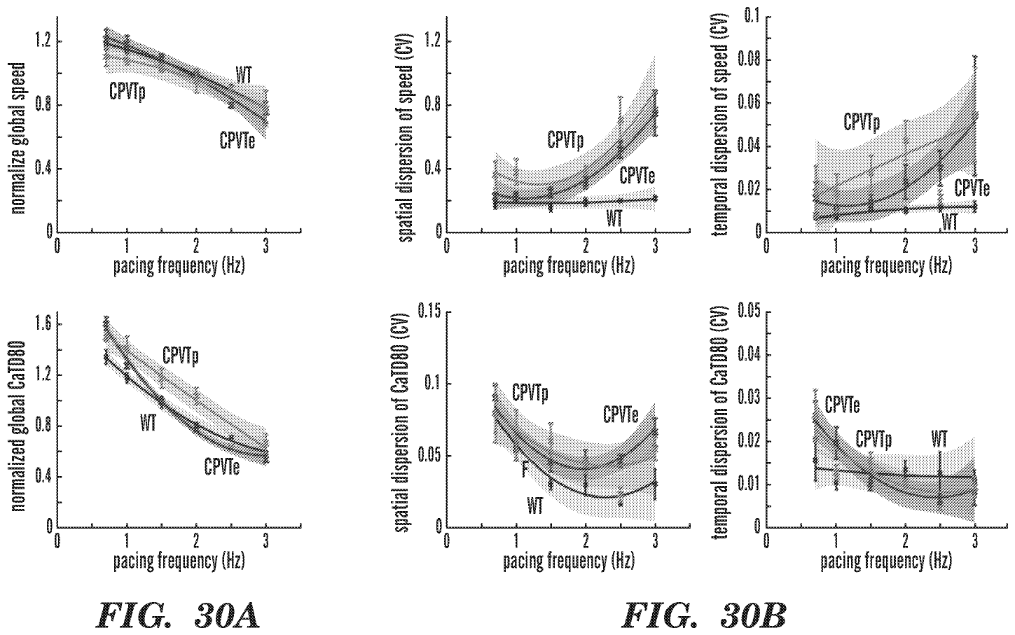

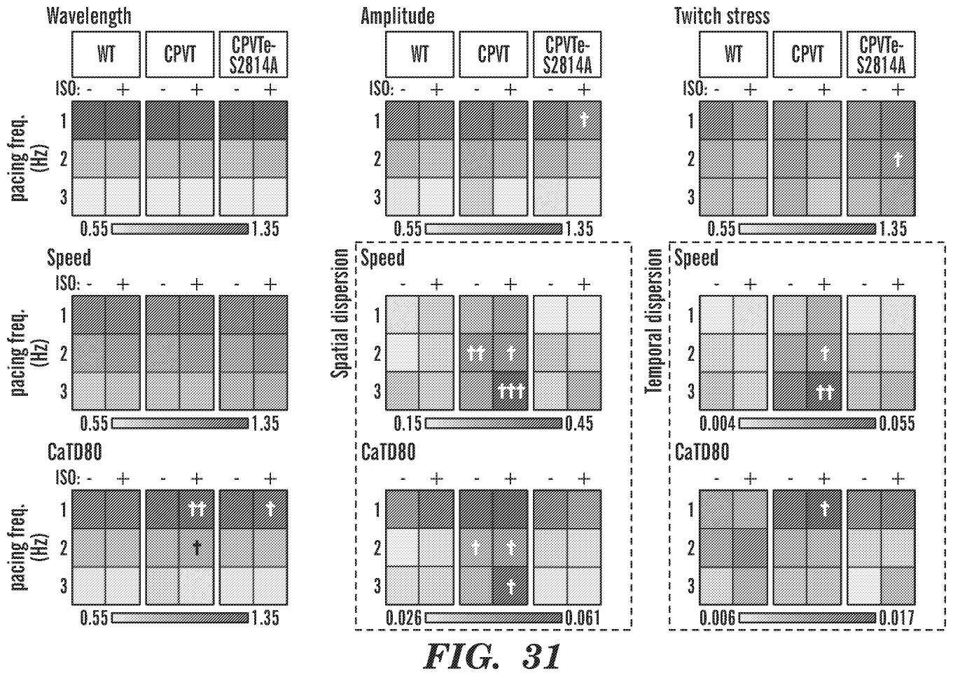

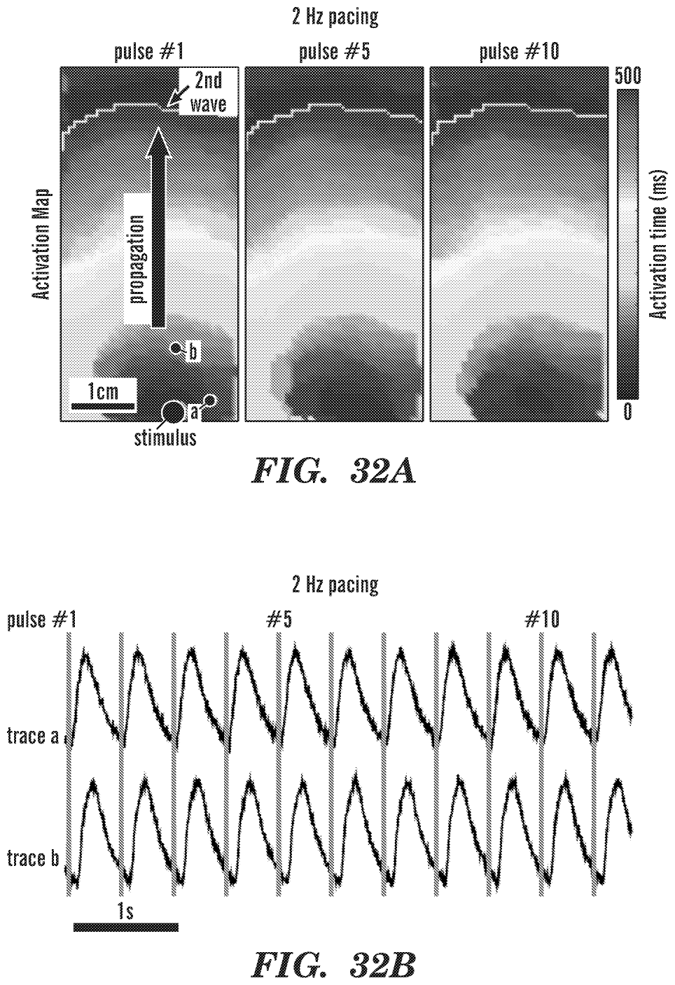

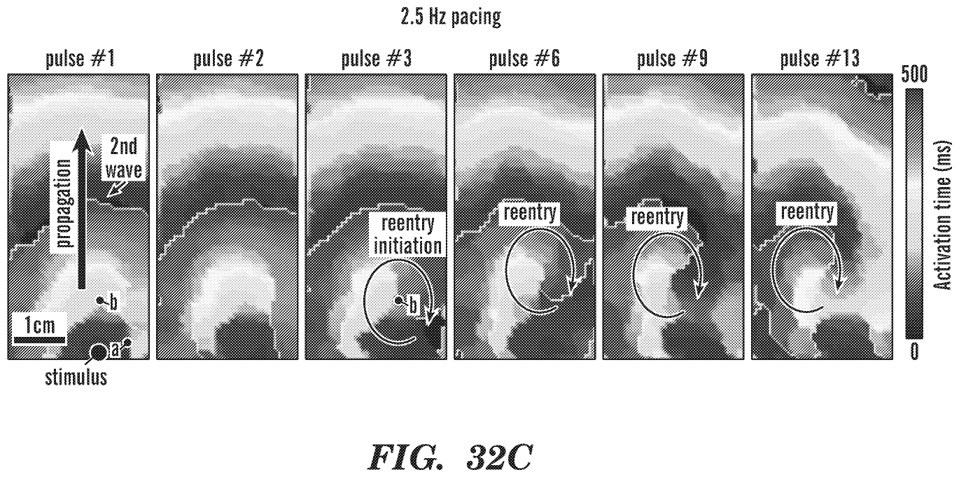

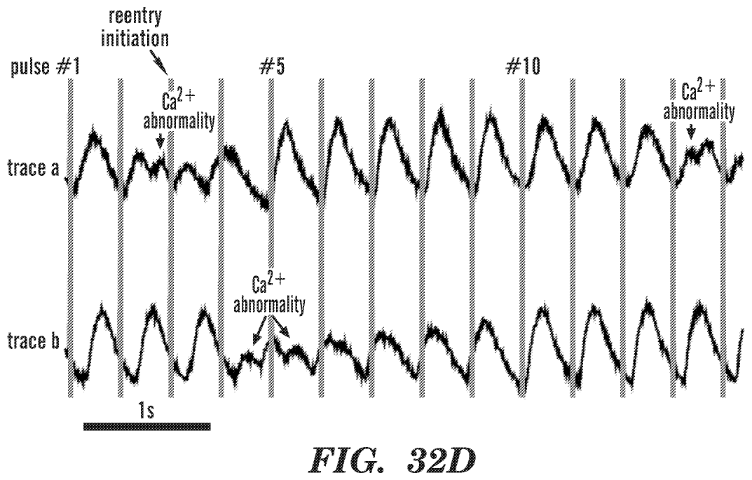

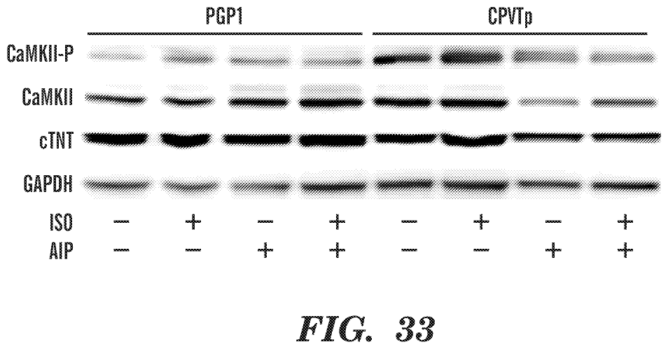

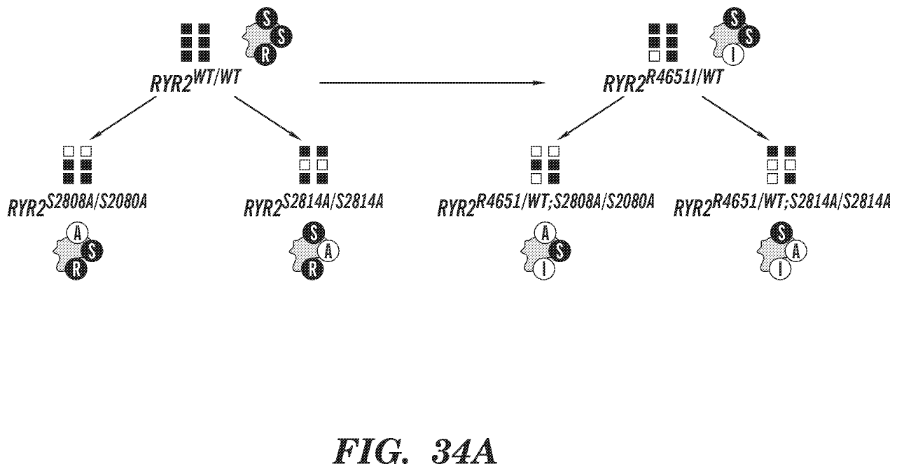

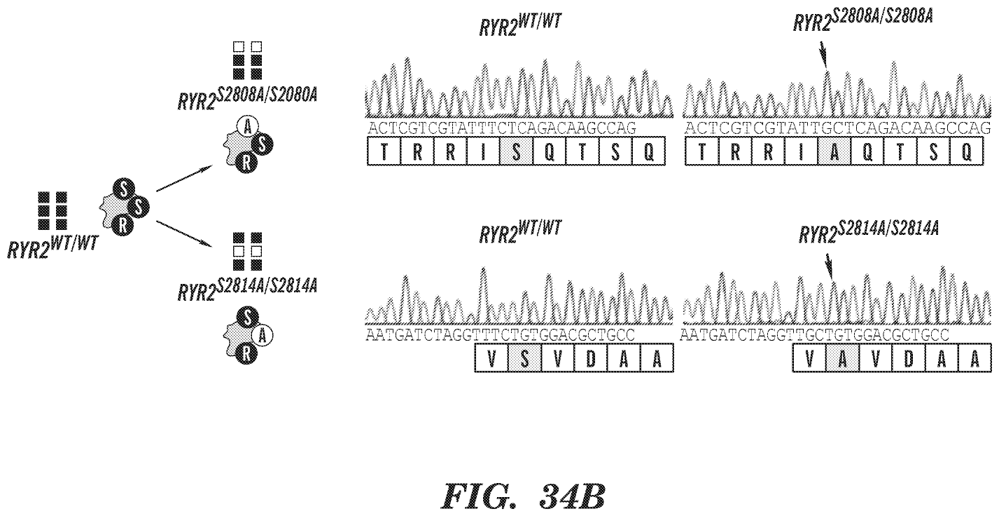

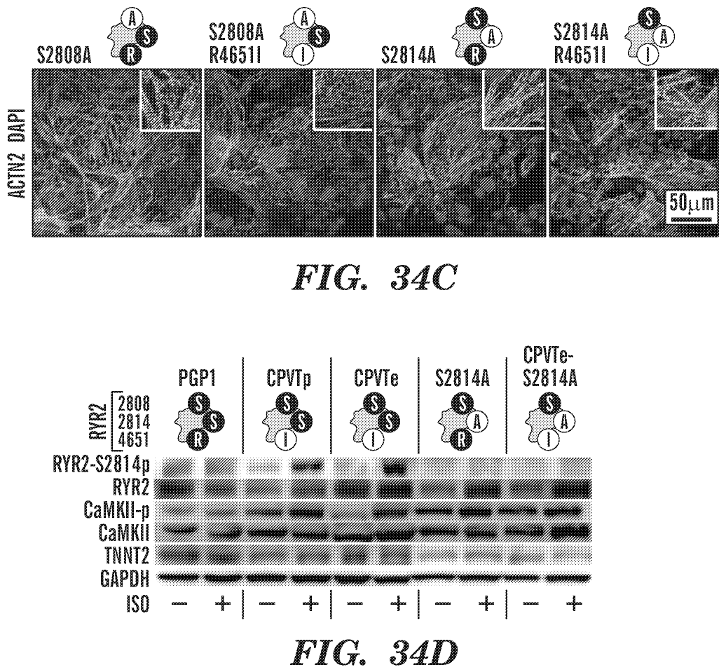

CaMKII targets multiple proteins that directly or indirectly impact Ca²⁺-handling. One important CaMKII target is serine 2814 (S2814) on RYR2 itself (FIG. 5B). RYR2-S2814 phosphorylation by CaMKII enhances diastolic RYR2 Ca²⁺ leak and is generally pro-arrhythmic. To test the hypothesis that CaMKII-mediated phosphorylation of RYR2-S2814 is essential for expression of CPVT mutations, Cas9 genome editing was used to replace S2814 with alanine (S2814A; FIG. 26) in both RYR2 alleles, in both RYR2 wild-type and RYR2^{R4651I/+} backgrounds. These mutant alleles are termed WT-S2814A and CPVTe-S2814A, respectively. RYR2 is also phosphorylated on 52808 by PKA, and in parallel genome editing was also used to generate the analogous RYR2-S2808A homozygous mutant lines, named WT-S2808A and CPVTe-S2808A (FIG. 26). In keeping with the effect of CaMKII inhibitory peptide, CPVTe-S2814A iPSC-CMs exhibited Ca²⁺ spark frequency that was lower than CPVTe and either comparable to WT, either at baseline or with isoproterenol stimulation (FIGS. 5C, 5D, and 26).

In contrast, CPVTe-S2808A iPSC-CMs had similar Ca²⁺ spark frequency compared to CPVTe (FIGS. 5C and 5D), consistent with the lack of effect of pharmacological PKA inhibition (FIG. 5A). Similar results were obtained by measuring the frequency of Ca²⁺ transients disrupted by after-depolarizations (FIGS. 5E and 5F). These data indicate CaMKII phosphorylation of RYR2-S2814 is required to unmask the pro-arrhythmic potential of the CPVT R4651I mutation.

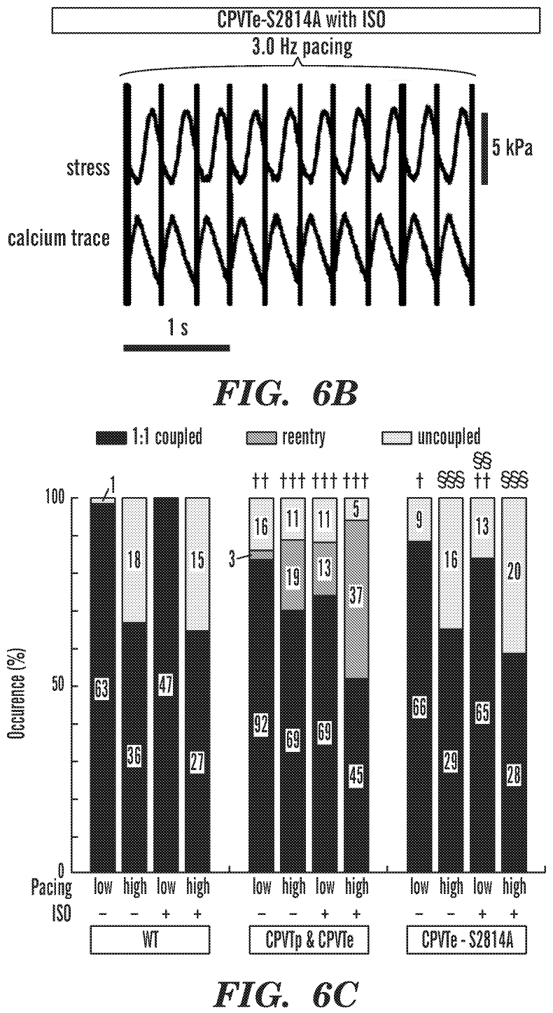

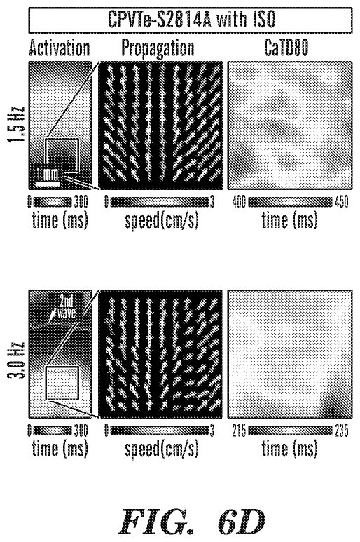

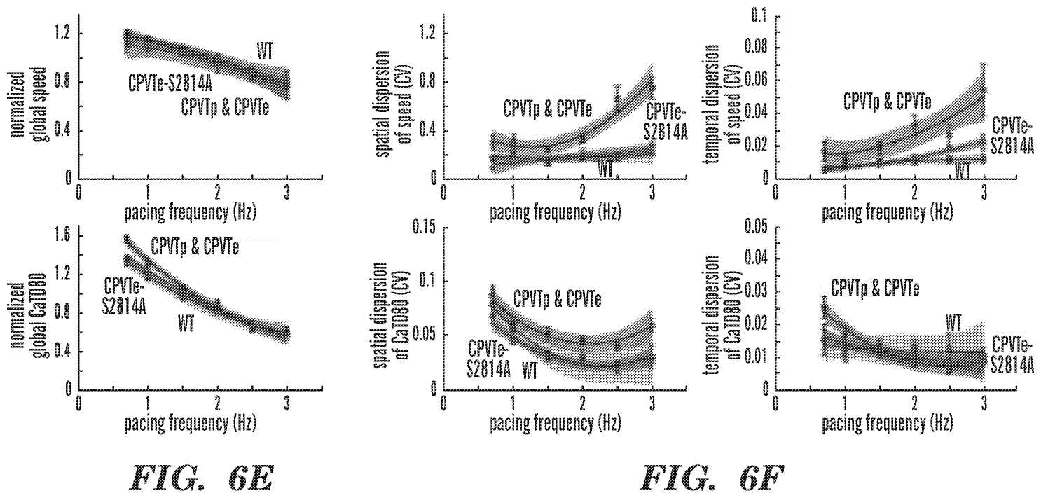

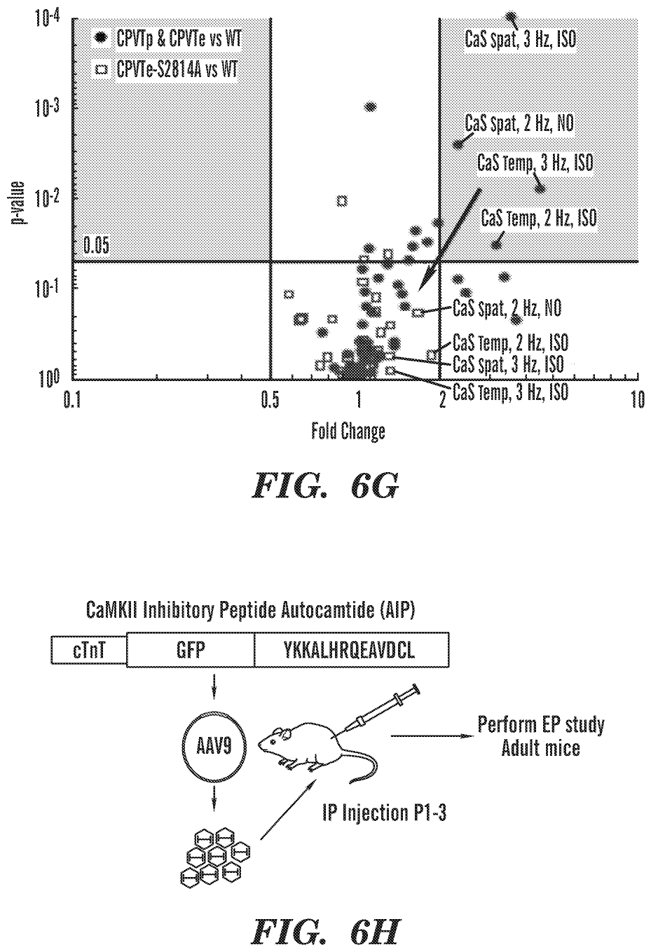

To model the effect of CaMKII inhibition on CPVT tissue, CPVTe or isogenic control opto-MTFs were treated with the selective inhibitor AIP. In both CPVTp and CPVTe opto-MTFs, AIP attenuated the frequency of spiral wave re-entry (data not shown). Next, opto-MTFs were fabricated from CPVTe-S2814A iPSC-CMs, which did not exhibit aberrant Ca²⁺ release in assays on cell islands. Rapid pacing and ISO did not induce re-entry in these tissues (FIGS. 6B-6D). Measurement of CV and CaTD dispersion in these tissues showed that abolishing 52814 phosphorylation prevented pacing- and ISO-induced increases in CPVT tissue heterogeneity (FIGS. 6D-6G). These data show that preventing RYR2-S2814 phosphorylation is sufficient to block tissue-level re-entry in CPVT.

A human tissue model of CPVT was created and used to elucidate the molecular and cellular pathogenesis of this disease. At a molecular level, CaMKII phosphorylation of RYR2-S2814 is required for full expression of the arrhythmic potential of the R4651I CPVT mutation. This phosphorylation event may be a cardiac selective therapeutic target for treatment of CPVT. At a tissue level, these studies indicate that re-entry is an important arrhythmia mechanism in CPVT. With rapid pacing and ISO stimulation, CPVT opto-MTFs developed greater tissue heterogeneity, resulting in a substrate vulnerable to re-entry. On this vulnerable substrate, sub-threshold after-depolarizations caused by the CPVT mutation initiate spiral wave re-entry.

### Example 2: AIP Inhibits Arrhythmia in a Murine Model of CPVT

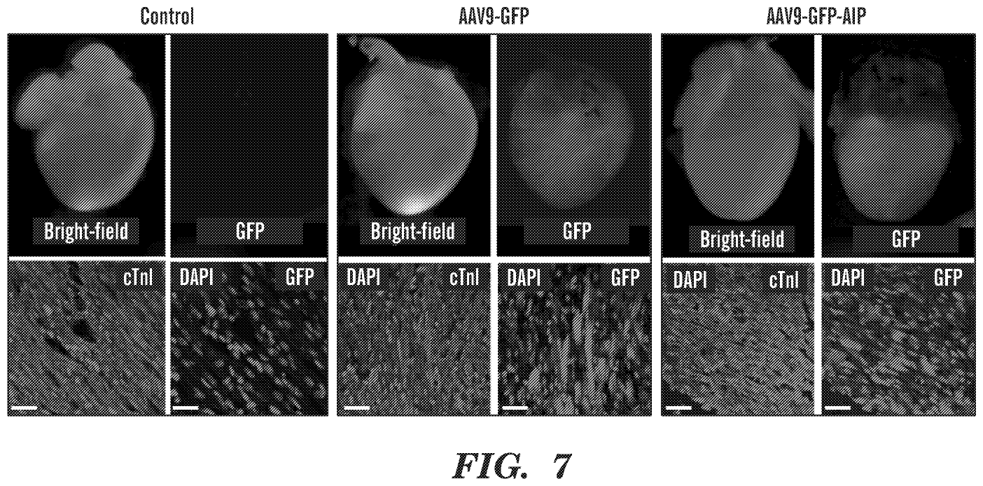

As reported herein above, AIP selectively inhibited CPVT in an opto-MTF model expressing the R4561I mutation. To determine efficacy in vivo, an adenoviral vector encoding a CaMKII Inhibitory Peptide Autocamtide (AIP) linked to GFP was generated. This adenoviral vector was injected into mice intraperitoneally (FIG. 6H). As shown in FIG. 7, AIP GFP expression was observed in murine cardiac tissues. Micrographs of cardiac tissue show the localization of AIP-GFP expression. About sixteen percent of troponin positive cells expressed low levels of GFP, while the vast majority of troponin positive cells expressed GFP at higher levels (FIG. 8, left panel). AIP expression in troponin positive cells was also quantitated (FIG. 8, right panel). With the majority of cells expressing AIP linked to GFP at a medium or high level.

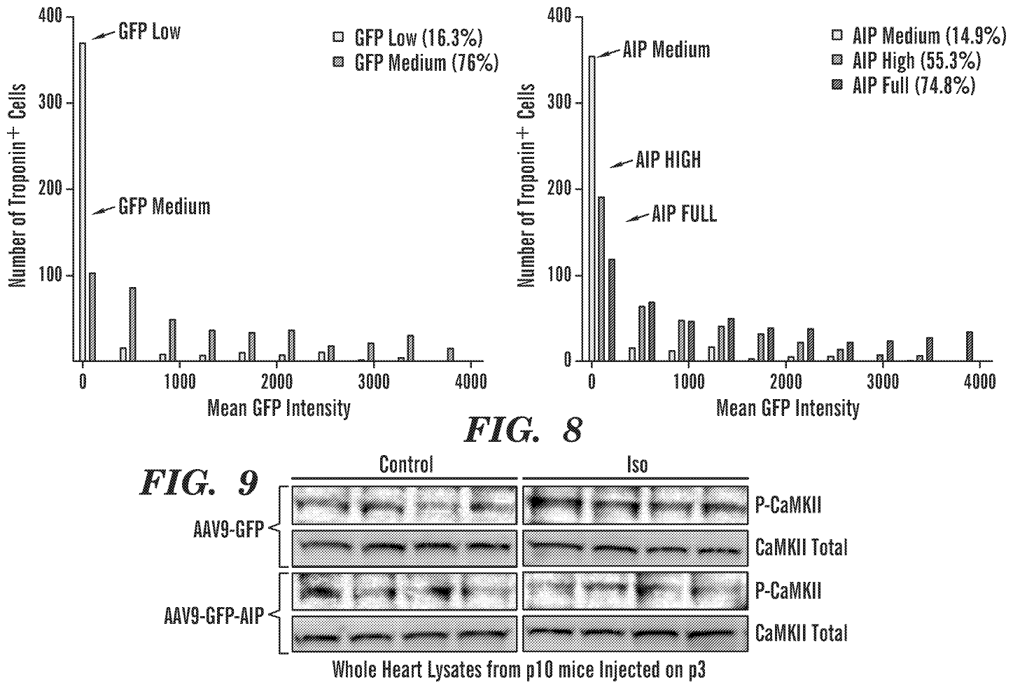

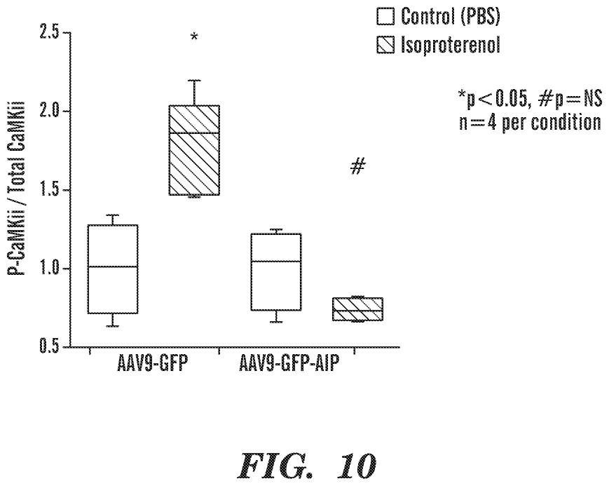

Expression of AIP was sufficient to inhibit phosphorylation by CaMKII in response to isoproterenol stimulation (FIGS. 9 and 10). Isoproterenol stimulation simulates key features of exercise induced CPVT. Levels of phosphorylated CaMKII in whole heart lysate was reduced in mice stimulated with isoproterenol that had been injected with the AIP expressing adenoviral vector, AAV9-GFP-AIP, relative to the level of phosphorylated CaMKII present in control lysates derived from mice injected with a control vector (FIGS. 9 and 10).

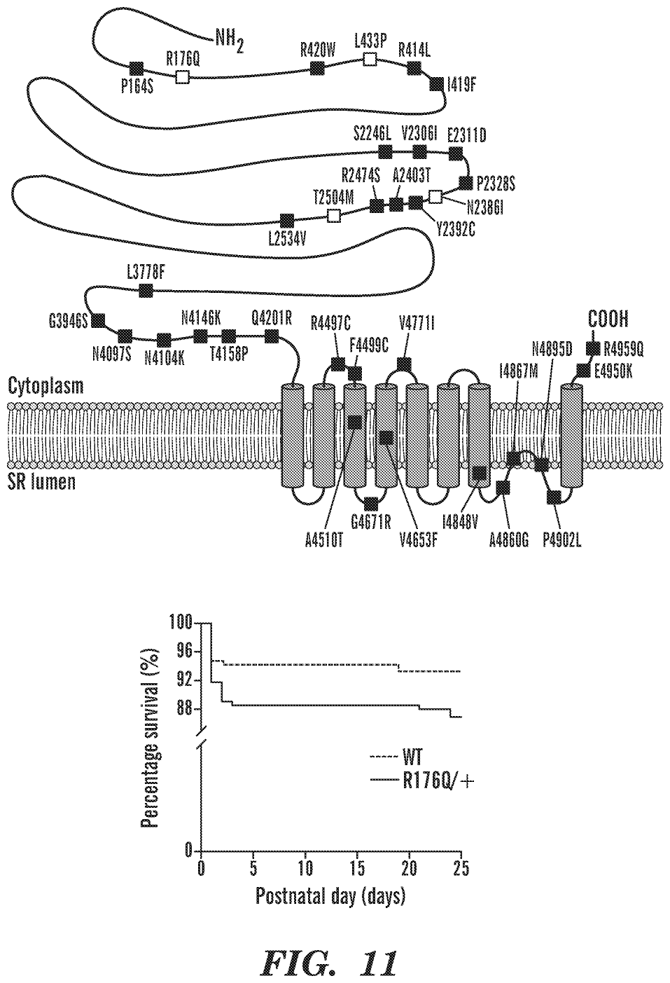

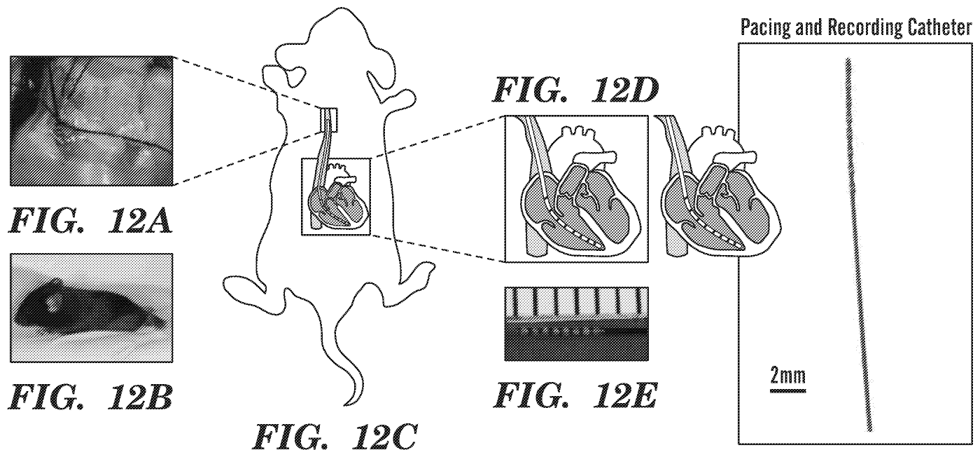

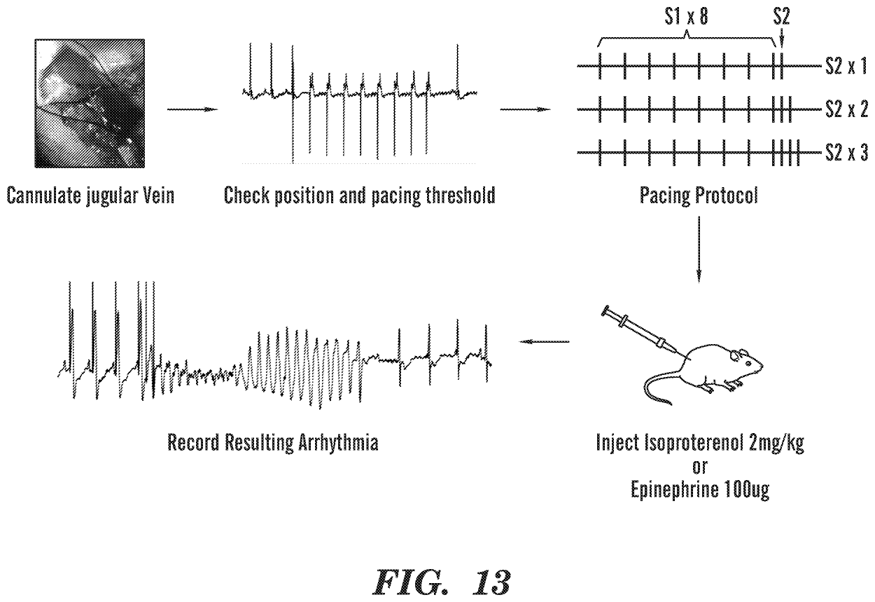

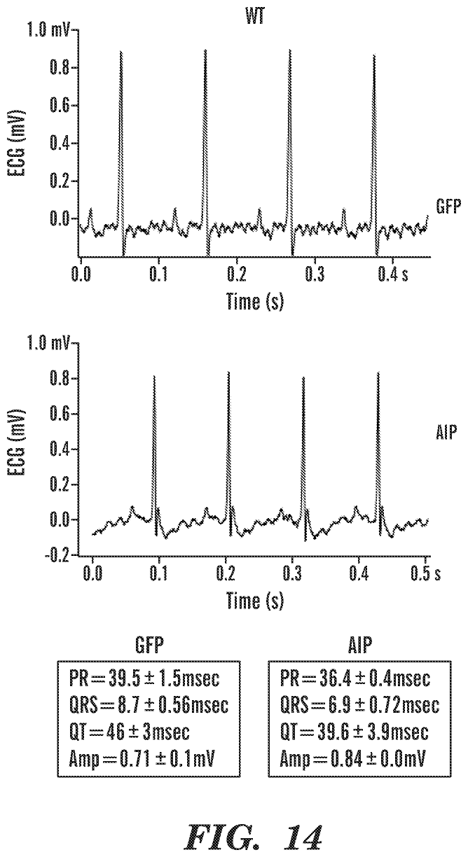

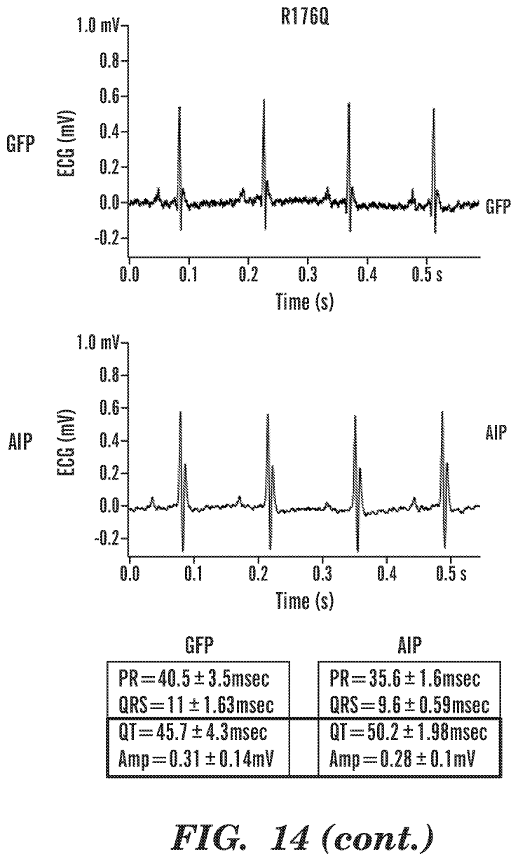

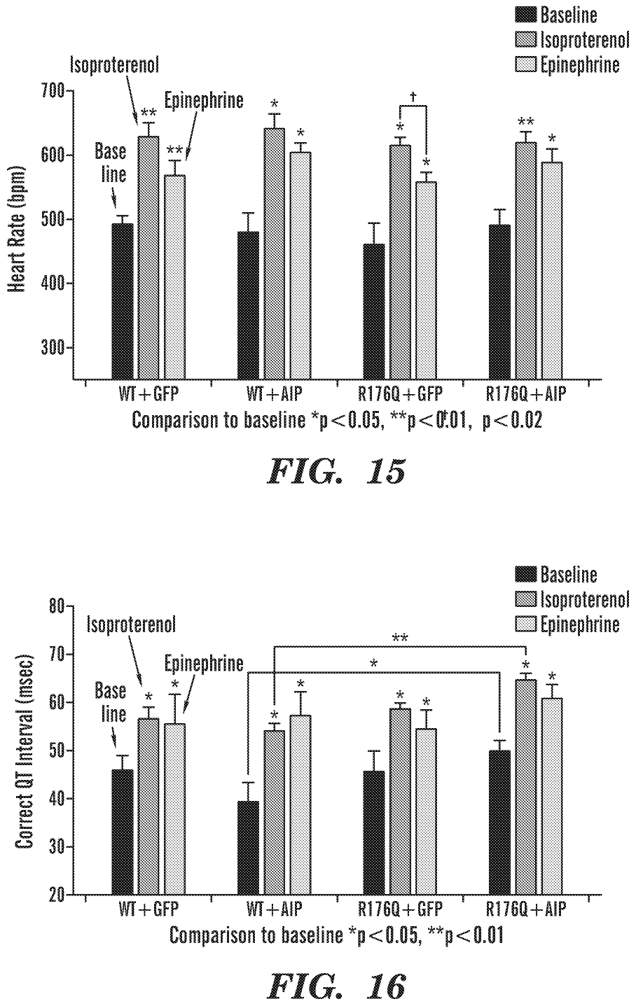

The role of phosphorylation in activating the cardiac ryanodine channel was further explored by generating a knock in of R176Q in the cardiac ryanodine channel (RYR2) (FIG. 11) and then characterizing the electrophysiology of mice carrying the R176Q mutation (FIGS. 12 and 13). Baseline electrocardiograms (ECGs) of wild type and mice having an R176Q mutation in the cardiac ryanodine channel (RYR2) are shown in FIG. 14. To mimic the effects of exercise induced CPVT, a pacing protocol and isoproterenol or epinephrine was used. Interestingly, the R176Q carrying mice that were treated with isoproterenol or epinephrine carrying the R176Q mutation showed changes in heart rate and baseline QT intervals relative to wild-type control mice (FIGS. 15 and 16). Changes in baseline and spontaneous arrhythmia in R176Q mice are shown in FIGS. 17A-17E. Induced arrhythmia was observed in R176Q mice (FIG. 18). These arrhythmias were not observed in R176Q mice that received a viral vector encoding AIP.

The results described herein above were obtained using the following methods and materials.

Human Fibroblast Cells Isolation and Reprogramming—Fresh skin biopsies from patients were cut into small pieces (less than 1 mm³) and incubated with collagenase 1 (1 mg/ml in DMEM) at 37° C. for 8 hours. The digested tissue from each patient was placed on tissue culture a dish, covered with a glass coverslip, and cultured in DMEM containing 10% FBS. After 7 days with daily media changes, fibroblast outgrowths on the tissue culture dish and coverslip were passaged. Fibroblasts were reprogrammed before passage 5 though episomal transfection with OCT4, SOX2, KLF4 and OCT4 expression constructs using Nucleofector™ Kits for Human Dermal Fibroblasts (Lonza). iPSCs were tested for pluripotency by qRTPCR and immunostaining of pluripotency genes, karyotyping, and in vivo teratoma formation.

Human iPSC Maintenance—All the IPSC lines in study were maintained in mTeSR™1 medium (STEMCELL Technologies) and passaged in versene solution (15040066, Thermo Fisher Scientific) every five days. Culture dishes were coated by 1:100 diluted Matrigel (Corning® Matrigel® hESC-Qualified Matrix, LDEV-Free) before passage.

Cardiomyocytes (iPSC-CMs) Differentiation from Human iPSCs—Human iPSC were seeded on Matrigel coated dishes in normal passage density. iPSC differentiation to iPSCCMs followed the timeline shown in FIG. 20E. On day 3 of iPSC culture, mTeSR™1 medium was removed, cells were rinsed once with PBS (without Ca²⁺ or Mg²⁺), and cultured in Differentiation Medium (RPMI medium (11875093, Thermo Fisher Scientific) with B27 without insulin (A1895601, Thermo Fisher Scientific)) containing 5 μM CHIR99021 (72054, STEMCELL Technologies). After 24 hours, medium was changed to differentiation medium without CHIR99021. At differentiation day 3, cells were cultured in differentiation medium containing 5 μM IWR-1 (3532, Tocris). After 48 hours, cells were cultured in differentiation medium without IWR until day 15, with media changes every 2-3 days. At day 15, the cells were cultured in Selection Medium (Non-Glucose DMEM (11966025, Thermo Fisher Scientific) with 0.4 mM Lactate (#L7022, Sigma Aldrich)) for 5 days to enrich for iPSCCMs.

### Differentiated Cardiomyocytes Isolation and Seeding on Engineering Chip

Human iPSC derived cardiomyocytes were isolated by incubating in collagenase 1 (Sigma C-0130, 100 mg collagenase 1 in 50 ml PBS/20% FBS) for 1 hour, followed by a 0.25% Trypsin incubation at 37° C. for 5-10 mins. 50% FBS in DMEM with 50 μg/ml DNase I (#260913, EMD Millipore) was used to stop trypsinization. The iPSC-CMs were suspended in Culture Medium (RPMI:Non-Glucose DMEM 1:1, plus 1× B27 without insulin and 0.2 mM Lactate) containing 10% FBS and 10 μM Y27632. The cardiomyocytes were suspended with culture medium contained 10% FBS and 10 μM Y27632 in final concentration as 1 million cells per 600 μl volume for engineering chip. After 48 hours, the medium was changed into chip culture medium (1:1 mixed by culture medium and selection medium). At the same time, the reseeded cardiomyocytes were infected with CHR2 lentivirus for 24 hours for future experiments.

Immunofluorescence Staining—Differentiated cardiomyocytes were seeded on Matrigel coated glass bottom dish for 5 days. The cells were fixed by 4% paraformaldehyde 10 min in room temperature, then 5% donkey serum plus 0.02% triton X-100 4° C. overnight permeabilized. The primary antibodies were used as 1:200 in 4° C. >8 h. Oct4 (SANTA CRUZ, SC8628), SSEA4 (Millipore, MAB4304), Cardiac Troponin I (Abcam, ab56357), ACTN2 (Abcam, ab56357), RYR2 (Abcam, ab2827). Imaging were taken by Olympus FV1000 confocal microscope.

Ca²⁺ Imaging—Differentiated cardiomyocytes were seeded on Matrigel coated glass bottom dish for 5 days. Every 50 μg Fluo 4 (F14201, Thermo Fisher Scientific) was dissolved with 8 ul of DMSO, then diluted 1:1 with Pluronic® F-127 (20% Solution in DMSO) (P3000MP, Thermo Fisher Scientific). The cardiomyocytes were treated with 3 ug/ml of Fluo 4 in 37° C. for half hour. Then washed with culture medium before Ca2+ recording. All the recording was recorded in culture medium. The recording was scanned by FV100 -Olympus confocal microscope in 10 ms/line and 1000 lines per recording. 10 μM KN93 (K1385 SIGMA) and 10 μM H89 DIHYDROCHLORIDE were used as CAMKII and PKA inhibit Compound, 0.025 μM Autocamptide-2 Related Inhibitory Peptide (SCP0001 SIGMA) and 1 μM PKA Inhibitor 14-22 (476485, EMD Millipore) were used as CAMKII and PKA inhibit peptide. Isoproterenol was used in 1 μM.

CRISPR/Cas9-Mediated Genome Editing—The procedures for CRISPR/Cas9 genome editing are known in the art. In general, wild-type PGP1 human iPSCs that contained doxycycline-inducible Cas9. Plasmid expressing guide RNA and 90 nucleotide donor oligonucleotide was transfected into the PGP1-Cas9 cells with Nucleofector™ Kits for Human Stem Cell (Lonza #VPH-5012) in the program B-016. Candidate clones from genome editing were PCR amplified and sequenced to verify that substitution mutation has occured. The sequencing primers as fellow:

For the site R4651:
R4651 forward primer:
(SEQ. ID. NO: 2)
TTG TAA GTT TAC GTG GCA GGA;
R4651 reverse primer:
(SEQ. ID. NO: 3)
CGC GTG CAT ATG TGT GTG TA;
For the site S2814:
S2814 forward primer:
(SEQ. ID. NO: 4)
ACACTATGTTTGGAAATTTGTGCCA;
S2814 reverse primer:
(SEQ. ID. NO: 5)
TGCTTTCCTGCATATATTTGGCA;
For the site S2808:
S2808 forward primer:
(SEQ. ID. NO: 6)
GGGCTGGAGAATTGAAAGAAC;
S2808 reverse primer:
(SEQ. ID. NO: 7)
CCCTTCTAAATTTTGTGACTCTTCA.
We selected heterozygous mutation in site R4651
and homozygous mutation in site S2814 and S2808.
The guide RNA sequences (gRNAs) used were:
For the site 2808:
(SEQ. ID. NO: 8)
CGTATTTCTCAGACAAGCCAGG
For the site 2814:
(SEQ. ID. NO: 9)
CAAATGATCTAGGTTTCTGTGG
For the site 4651:
(SEQ. ID. NO: 10)
GACAAATTTGTTAAAATAAAGG
The 90 nucleotide Homology-directed repair (HDR)
template were:
For the site 2808:
(SEQ. ID. NO: 11)
CGGGAGGGAGACAGCATGGCCCTTTACAACCGGACTCGTCGTATTGCTC
AGACAAGCCAGGTAAGAATTCATCACGGTGATGAATCAACTG
For the site 2814:
(SEQ. ID. NO: 12)
AGGTTTTTAATGAGGCACTGTTTTTTCACACAAATGATCTAGGTTGCTG
TGGACGCTGCCCATGGTTACAGTCCCCGGGCCATTGACATGA
For the site 4651:
(SEQ. ID. NO: 13)
ATTTTAGGTCATTTCCCAACAACTACTGGGACAAATTTGTTAAAATAAA
GGTAATATTACTTGGAATCCTCTACATTTTTCTTAAAGCACA

Culture of Commercial iPSC-CMs—Commercial hiPSC derived cardiomyocytes (hiPSC-CMs, Cor4U; Axiogenesis, Cologne, Germany) were cultured according to manufacturer's instructions. Briefly, a T-25 cell culture flask (per each 1-million cryovial) was coated with 0.01 μg/mL fibronectin (FN) (BD Biosciences, Bedford, Mass.) one day before the cell seeding. Cryovials were quickly thawed in a 37° C. water bath and resuspended in 9 mL of complete culture media (Axiogenesis, Cologne, Germany) supplemented with 4.5 μL of 10 mg/mL puromycin (Axiogenesis, Cologne, Germany). After 24 hours, the cell culture media were replaced with puromycin free media (total volume 10 ml). After 48 hours, the cells were dissociated with 0.25% trypsin-EDTA (Life Technologies) for 10 min, and then washed and suspended in puromycin free media. The resuspended cells were used for seeding coverships or opto-MTF chips.

Neonatal Rat Ventricular Myocyte Harvest—The neonatal rat ventricular myocyte isolation was performed as previously described in the art. Briefly, ventricles were removed from 2-day old Sprague Dawley rat pups (Charles River Laboratories). The tissue was manually minced. For the first enzymatic digestion, the tissue was placed in a 0.1% trypsin (Sigma Aldrich) solution at 4° C. for approximately 12 hours. For the second stage of enzymatic digestion, the trypsin was replaced with a 0.1% type II collagenase (Sigma Aldrich) solution. After four iterations of the second stage digestions at 37° C., ventricular myocytes were further isolated from the resulting dissociated cell solution by centrifuging and passing the resuspended solution through a 40 μm cell strainer. The solution was pre-plated twice for 45 minutes each at 37° C. to remove fibroblasts and endothelial cells. Then, we created the seeding solution by resuspending the resulting ventricular myocytes in a M199 cell media (Life Technologies) supplemented with 10% heat-inactivated FBS (Life Technologies).

Gelatin muscular thin film (MTF) substrate fabrication Glass coverslips (22 by 22 mm square) were cleaned using 70% ethanol (Sigma) and were then covered with low adhesive tape (3M). Using a laser engraving system (Epilog Laser), the tape was cut to have two rectangles in the center, surrounded by four trapezoids on the outer edges. The inner rectangles of 3 mm by 10 mm and 7 mm by 10 mm are for the cantilever and base region of the MTFs respectively.

Glass coverslips were selectively activated, such that the gelatin in the base region of MTFs would firmly attach to the glass coverslips but the gelatin in the cantilever region would be easily peeled. Firstly, only the base region tape was removed, while the tapes in the cantilever and outer regions remained to protect the glass from the following activation. The coverslips were activated with a 0.1 M NaOH (Sigma) solution for 5 minutes, a 0.5% APTES (Sigma) solution in 95% ethanol (Sigma) for 5 minutes, followed by a 0.5% glutaraldehyde solution for 30 minutes.

The tape in the cantilever region was removed after the activation process, but the tapes in outer regions remained on the glass coverslips. 20% w/v gelatin (Sigma) and 8% w/v MTG (Ajinomoto) were warmed to 65° C. and 37° C., respectively for 30 minutes. Then, the solutions were mixed to produce a final solution of 10% w/v gelatin and 4% w/v MTG. 300 μl of the gelatin mixture was quickly pipetted onto the exposed inner rectangle regions of glass coverslips. PDMS stamps with line groove features (25 μm ridge width, 4μm groove width, and 5 μm groove depth) were then inverted on top of the gelatin drop and weight was applied using a 200 g weight. Gelatin was then left to cure overnight at room temperature with the stamp and the weight in place.

After the gelatin cured, the weight was carefully removed along with excess gelatin on the sides of the stamp. To minimize damage to the micro-molded gelatin, the coverslip and stamp were immersed in distilled water to rehydrate the gelatin for an hour. The stamp was then carefully peeled off the gelatin.

Coverslips with the micro-molded gelatin were quickly dried with paper wipes (Kimwipes, Kimberly-Clark Professional). Cantilevers (1 mm wide×2 mm long) were laser engraved into the dehydrated micro-molded gelatin using an Epilog laser engraving system with 3% power, 7% speed, and a frequency of 1900 Hz. Gelatin chips were UVO-treated for 90 seconds and re-hydrated in a 2 mM MES solution of pH 4.5 with 1 mg/ml collagen and 0.1 mg/mg fibronectin. The gelatin chips were stored in solution at room temperature for 2 hours. The collagen and fibronectin solution was replaced with PBS. The gelatin chips were stored at 4° C. until cell seeding.

Soft lithography and PDMS micromolded stamp fabrication - Micro-molded stamps were fabricated from polydimethylsiloxane (PDMS, Sylgard 184, Dow Corning) using previously published soft lithography protocols that are known in the art. Briefly, 5 μm thick SU-8 2005 photoresist (MicroChem) was spin-coated on silicon wafers and prebaked at 90° C. as suggested in the MicroChem protocol manual. The SU-8 layer was exposed to UV light under customized photomasks with line features (25 μm wide dark lines and 4 μm wide clear lines). After exposure, wafers were post-baked at 90° C., developed with propylene glycol monomethyl ether acetate, and silanized with fluorosilane (United Chemical Technologies). PDMS was mixed at 10:1 base to curing agent ratio, poured onto the wafer, cured at 65° C. for 4 hours, carefully peeled from the wafer, and cut into micromolded stamps.

Opto-MTF Construction—ChR2 lentiviral vector in which the cardiac troponin T promoter drives ChR2-eYFPP was constructed based on the FCK(1.3)GW plasmid with the cardiac troponin T (cTnT) promoter, ChR2, and enhanced yellow fluorescent tag.

Prior to seeding, the gelatin chips were washed with PBS and incubated with hiPSC-CM or NRVM seeding media. Dissociated WT, CPVTp, and CPVTe iPSC-CMs were suspended in culture medium media containing 10% FBS and 10 μM Y27632 at a final concentration of 1 million cells per 600 μl. After 48 hours, the culture media was replaced with Chip Culture Medium (1:1 mix of Culture Medium and Selection Medium). At the same time, the iPSC-CMs were transduced with ChR2 lentivirus at a multiplicity of infection of 14-23 for 24 hours. Commercial hiPSC-CMs (Cor4U; Axiogenesis, Cologne, Germany) and NRVM cells were seeded onto devices at a density of 220 k/cm² and 110 k/cm², respectively. After 24 hours, the NRVMs were treated with ChR2 lentivirus at multiplicity of infection of 14-23 for 24 hours.

Immunofluorescent Staining of Engineered Cardiac Tissues on Micromolded Gelatin Hydrogels—iPSC-CM opto-MTFs were washed with PBS at 37° C., fixed in PBS with 4% paraformaldehyde and 0.05% Triton X-100 for 12 mins at 37° C., and rinsed with PBS. Tissues were stained with mouse anti-sarcomeric a-actinin monoclonal primary antibody (Sigma) for 1 hour at room temperature, and then with a secondary antibody against mouse IgG conjugated to Alexa-Fluor 546 (Life Technologies) and DAPI (Life Technologies). The samples were mounted on glass slides with ProLong Gold antifade mountant (Life Technologies). Z-stack images were acquired using a confocal microscope (Zeiss LSM) equipped with an alpha Plan-Apochromat 100×/1.46 Oil DIC M27 objective.

Western Blot—10% Invitrogen Bolt gels were used to run all the samples. For RYR2 and RYR2-P2814 western blots, transfer was performed using 75V for 900 minutes. Other westerns were transferred using 80V for 120 min. The antibody antibodies used for western blots were as follows: CaMKII-phospho-T286 (Abcam, ab171095), CaMKII (Abcam, ab134041), RYR2-phospho-S2814 (Badrilla A010-31AP), and Cardiac Troponin T (Abcam ab45932). HiMark Pre-Stained Protein Standards (Life Technologies #LC5699) was used as molecular weight markers.

Ca²⁺ imaging of cell clusters - iPSC-CMs were seeded on Matrigel-coated glass bottom dishes for 5 days. 50 μg of Fluo-4 (F14201, Thermo Fisher Scientific) was dissolved in 8 μl of DMSO, then diluted 1:1 with Pluronic® F-127 (20% Solution in DMSO) (P3000MP, Thermo Fisher Scientific). The iPSC-CMs were treated with 3 μg/ml Fluo-4 at 37° C. for a half hour. The samples were then washed with Culture Media before Ca²⁺ imaging on an Olympus FV1000 using line scan mode (10 msec/line, 1000 lines per recording). The scan line was positioned within individual iPSC-CMs that belonged to clusters of 3-10 cells. Recordings of spontaneous Ca²⁺ release events were made during periods when cells did not exhibit spontaneous Ca²⁺ transients, or during periods of spontaneous beating. 0.025 μM myristolated Autocamtide-2-related Inhibitory Peptide (SCP0001 Sigma) and 1 μM PKA Inhibitor 14-22 amide (476485, EMD Millipore) were used as CaMKII and PKA inhibiting peptides. Isoproterenol was used at 1 μM.

Optical Setup for Opto-MTF—Tandem-lens macroscope (Scimedia) was modified the for simultaneous Ca²⁺ imaging and contractility measurement with optogenetic stimulation (FIG. 20). For Ca²⁺ imaging, the system was equipped with a highspeed camera (MiCAM Ultima, Scimedia), a plan APO lx objective, a collimator (Lumencor) and a 200 mW mercury lamp for epifluorescence illumination (X-Cite exacte, Lumen Dynamics). For contractility measurements, a high-spatial resolution sCMOS camera (pco.edge, PCO AG) and 880 nm darkfield LED light (Advanced Illumination) were incorporated into the system. The field of view of the system for Ca²⁺ and dark field imaging was 10 mm by 10 mm and 16 mm by 13 mm, respectively. For optogenetic stimulation, an 8 channel LED array (465/25 nm, Doric Lenses) was used to generate optical pulses. Light pulses for pacing individual MTFs were delivered through the 8 optical fibers (400 μm diameter, NA 0.48, Doric Lenses) and 8 mono fiber optic cannulas (flat end, 400 μm diameter, NA 0.48, Doric Lenses) mounted 500 μm above the gelatin chips using a 3-axis manipulator (Zaber, Canada). To prevent overlap of the excitation light wavelength for Ca²⁺ transients and dark field illumination for contractility measurements with the ChR2 excitation wavelength, a filter set with longer wavelengths than the ChR2 excitation wavelength was used. For Ca²⁺ imaging, an excitation filter with 580/14 nm, a dichroic mirror with 593 nm cut-off, and an emission filter with 641/75 nm (Semrock, Rochester, NY) were used. For dark field imaging, a dichroic mirror with 685 nm cut-off and long pass emission filter with 664 nm cut-off (Semrock, Rochester, N.Y.) were added into the light path for Ca²⁺ imaging. The light sources of the LED array were independently controlled by analog signals that were synthesized with an analog output module (NI 9264, National Instruments) by custom software written in LabVIEW (National Instruments). For post-imaging processing, these analog signals were recorded using a high-speed camera and a high-spatial resolution sCMOS camera simultaneously, to use the analog signals as a reference for aligning frames from both systems.

Tissue Level Data Acquisition—At post-transduction day 3, engineered opto-MTF tissues were incubated with 2 μM X-Rhod-1(Invitrogen, Carlsbad, CA) for 30 min at 37° C., rinsed with culture medium with 2% FBS to remove nonspecifically associated dye, and incubated again for 30 mins to complete de-esterification of the dye. Prior to recording for the experiments, the culture media was replaced with Tyrode's solution (1.8 mM CaCl₂, 5 mM glucose, 5 mM Hepes, 1 mM MgCl₂, 5.4 mM KCl, 135 mM NaCl, and 0.33 mM NaH₂PO₄ in deionized water, pH 7.4, at 37° C.; Sigma). The engineered tissue sample in Tyrode's solution was maintained at 37° C. during the experiments using a culture dish incubator (Warner Instruments).

The engineered opto-MTF tissues were stimulated with an optical pulse of 10 ms over a range of frequencies from 0.7 to 3 Hz using a custom LabVIEW program (National Instruments). The optical point stimulation was applied at one end of the MTF tissue using an LED light source (465/25 nm, Doric Lenses). For each recording, Ca²⁺ and dark field images were simultaneously acquired with 2000 frames and 400 frames at a frame rate of 200 Hz and 100 Hz over 10 s and 4 s, respectively.

Analysis of Calcium Imaging Data—Post-processing of the raw calcium data was conducted with custom software written in MATLAB (MathWorks). A spatial filter of 3×3 pixels was applied to improve the signal-noise ratio. First, local Ca²⁺ activation time, Tact_{p,px} and 80% repolarization time, CaTD80_{p,px} of each pixel (px) and each pulse (p) was calculated by identifying the time with the maximum upstroke slope and the time from the upstroke to 80% recovery, respectively. Then, the calcium propagation speed, CaS_{p,px} of each pixel and each pulse was determined by calculating the x- and y directional change rate of Tact_{p,px} in 21 pixels (3 pixels in the transverse direction, x, and 7 pixels in the longitudinal direction of the wave, y). To calculate the spatial dispersions of the Ca²⁺ propagation speed, CaSₛₚₐₜ and 80% repolarization time, CaTD80ₛₚₐₜ, we averaged CaS_{p,px} and CaTD80_{p,px} over multiple consecutive pulses (3-20 pulses) of each pixel and calculated the coefficient of variance of these temporal averages over an area of interest (500 to 1000 pixels). To calculate the temporal dispersions of calcium propagation speeds, CaSₜₑₘₚ and 80% repolarization time, CaTD80ₜₑₘₚ, we averaged CaS_{p,px} and CaTD80_{p,px} over all areas of interest of each pulse and calculated the coefficient of variance of these spatial averages over multiple consecutive pulses. The global Ca²⁺ propagation speed, CaS_{global} and 80% repolarization time, CaTD80_{global}, were calculated by averaging CaS_{p,px} and CaTD80_{p,px} over multiple consecutive pulses and pixel areas of interest. Regions where local Ca²⁺ propagation speed was less than 0.2 cm/s were defined as having functional conduction block. In addition, we measured global Ca²⁺ wavelength, Ca²⁺ signal amplitude, and relative diastolic Ca²⁺ level. The global calcium wavelength was defined as the distance traveled by the waves during the duration of the calcium refractory period and calculated by multiplying calcium propagation speed, CaS_{global} and 80% repolarization time, CaTD80_{global}. The calcium amplitude was calculated as a difference between peak systolic and diastolic Ca²⁺ level. Relative diastolic Ca²⁺ levels were calculated from the mean diastolic value at more than 500 sampling points distributed throughout the tissue by subtracting the background intensity measured at 10 points outside the opto-MTF. This background-subtracted value at the base rate (0.7 Hz, no ISO) was set as FO. The change in relative diastolic Ca²⁺ level at higher pacing frequencies was calculated as (F-F0)/F0. To determine the ISO and pacing frequency-dependence of global variables, global variable data were normalized to values from the same opto-MTF at 1.5 Hz pacing without ISO.

Analysis of Contractility Dark Field Imaging Data—Post-processing of the dark field imaging data was conducted using custom software written in MATLAB (MathWorks). The contractile stress quantification ImageJ software program was modified as known in the art. First, the projected length of each MTF from each frame was measured by using image thresholding MATLAB functions. Then, the film stress was calculated using the projected length, gelatin film thickness, and gelatin properties by considering the geometric relationship of the radius of curvature, the angle of the arc, and the projected length of the film, using a modified Stoney's equation. Here, the Young's modulus=56 kPa, and gelatin MTF thickness=188 μm, as previously determined in the art. Twitch stress was calculated as the difference between peak and baseline stresses.

Statistical Analysis—Tissue-level functional differences were calculated with Student's t-test (p<0.05) and Benjamini-Hochberg multiple testing correction was applied with false discovery rate (FDR) of 20%.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A pharmaceutical composition comprising an effective amount of a vector encoding a CaMKII peptide inhibitor.

2. The composition of claim 1, wherein the CaMKII peptide inhibitor is AIP, CN19, CN19o, CN27, CN21, or an analog or fragment thereof.

3. An expression vector comprising a polynucleotide encoding a CaMKII peptide inhibitor.

4. The expression vector of claim 3, wherein the CaMKII peptide inhibitor is operably linked to a promoter suitable for driving expression of the peptide in a mammalian cardiac cell.

5. The expression vector of claim 3, wherein the vector is a pharmaceutical composition comprising an effective amount of an CaMKII peptide inhibitor, analog, or fragment thereof.

6. The expression vector of claim 3, wherein the vector is a retroviral, adenoviral, or adeno-associated viral vector.

7. A cell comprising the expression vector of claim 3.

8. A method for modulating a cardiac arrhythmia in a subject, the method comprising contacting a cell comprising a cardiac ryanodine channel (RYR2) with a CaMKII inhibitor, CaMKII peptide inhibitor or polynucleotide encoding the CaMKII peptide inhibitor.

9. A method for inhibiting the phosphorylation of a ryanodine channel (RYR2) polypeptide in a subject, the method comprising contacting a cell comprising a cardiac ryanodine channel (RYR2) with a CAMKII inhibitor, CaMKII peptide inhibitor or polynucleotide encoding a CaMKII peptide inhibitor.

10. A method of treating a subject comprising a mutation associated with a cardiac arrhythmia, the method comprising administering to the subject a CaMKII inhibitor, CaMKII peptide inhibitor, analog, or fragment thereof or polynucleotide encoding a CaMKII peptide inhibitor.

11. The method of claim 10, wherein the mutation is in a cardiac ryanodine channel (RYR2).

12. The method of claim 11, wherein the mutation is RYR2^{R4651I}.

13. The method of claim 8, wherein the method inhibits a cardiac arrhythmia.

14. The method of claim 8, wherein the method inhibits catecholaminergic polymorphic ventricular tachycardia in the subject.

15. A method of characterizing a cardiomyocyte, the method comprising monitoring cardiac conduction or contraction using an induced pluripotent stem cell derived cardiomyocyte expressing a cardiac ryanodine channel (RYR2) comprising a mutation associated with catecholaminergic polymorphic ventricular tachycardia (CPVT).

16. A method of compound screening, the method comprising contacting an induced pluripotent stem cell derived cardiomyocyte expressing a cardiac ryanodine channel (RYR2) comprising a mutation associated with catecholaminergic polymorphic ventricular tachycardia (CPVT) with a candidate agent and measuring cardiac conduction or contraction in the cell.

17. The method of claim 9, wherein the method inhibits a cardiac arrhythmia.

18. The method of claim 10, wherein the method inhibits a cardiac arrhythmia.

19. The method of claim 9, wherein the method inhibits catecholaminergic polymorphic ventricular tachycardia in the subject.

20. The method of claim 10, wherein the method inhibits catecholaminergic polymorphic ventricular tachycardia in the subject.


SEQUENCE LISTING (28 sequences)

SEQ ID NO: 1 - protein, 13 aa, Artificial Sequence
  Description of Artificial Sequence Synthetic peptide
KKALRRQEAVDAL

SEQ ID NO: 2 - DNA, 21 bp, Artificial Sequence
  Description of Artificial Sequence Synthetic primer
ttgtaagtttacgtggcagga

SEQ ID NO: 3 - DNA, 20 bp, Artificial Sequence
  Description of Artificial Sequence Synthetic primer
cgcgtgcatatgtgtgtgta

SEQ ID NO: 4 - DNA, 25 bp, Artificial Sequence
  Description of Artificial Sequence Synthetic primer
acactatgtttggaaatttgtgcca

SEQ ID NO: 5 - DNA, 23 bp, Artificial Sequence
  Description of Artificial Sequence Synthetic primer
tgctttcctgcatatatttggca

SEQ ID NO: 6 - DNA, 21 bp, Artificial Sequence
  Description of Artificial Sequence Synthetic primer
gggctggagaattgaaagaac

SEQ ID NO: 7 - DNA, 25 bp, Artificial Sequence
  Description of Artificial Sequence Synthetic primer
cccttctaaattttgtgactcttca

SEQ ID NO: 8 - DNA, 22 bp, Artificial Sequence
  Description of Artificial Sequence Synthetic oligonucleotide
cgtatttctcagacaagccagg

SEQ ID NO: 9 - DNA, 22 bp, Artificial Sequence
  Description of Artificial Sequence Synthetic oligonucleotide
caaatgatctaggtttctgtgg

SEQ ID NO: 10 - DNA, 22 bp, Artificial Sequence
  Description of Artificial Sequence Synthetic oligonucleotide
gacaaatttgttaaaataaagg

SEQ ID NO: 11 - DNA, 91 bp, Artificial Sequence
  Description of Artificial Sequence Synthetic oligonucleotide
cgggagggagacagcatggccctttacaaccggactcgtcgtattgctcagacaagccaggtaagaattcatcacggtgatgaatcaactg

SEQ ID NO: 12 - DNA, 91 bp, Artificial Sequence
  Description of Artificial Sequence Synthetic oligonucleotide
aggtttttaatgaggcactgttttttcacacaaatgatctaggttgctgtggacgctgcccatggttacagtccccgggccattgacatga

SEQ ID NO: 13 - DNA, 91 bp, Artificial Sequence
  Description of Artificial Sequence Synthetic oligonucleotide
attttaggtcatttcccaacaactactgggacaaatttgttaaaataaaggtaatattacttggaatcctctacatttttcttaaagcaca

SEQ ID NO: 14 - protein, 14 aa, Artificial Sequence
  Description of Artificial Sequence Synthetic peptide
YKKALHRQEAVDCL

SEQ ID NO: 15 - DNA, 27 bp, Homo sapiens
aaatttgttaaaagaaaggtaatatta

SEQ ID NO: 16 - protein, 6 aa, Homo sapiens
KFVKRK

SEQ ID NO: 17 - DNA, 27 bp, Artificial Sequence
  Description of Artificial Sequence Synthetic oligonucleotide
aaatttgttaaaakaaaggtaatatta

SEQ ID NO: 18 - protein, 6 aa, Artificial Sequence
  Description of Artificial Sequence Synthetic peptide
KFVKAK

SEQ ID NO: 19 - DNA, 27 bp, Artificial Sequence
  Description of Artificial Sequence Synthetic oligonucleotide
aaatttgttaaaataaaggtaatatta

SEQ ID NO: 20 - protein, 6 aa, Artificial Sequence
  Description of Artificial Sequence Synthetic peptide
KFVKIK

SEQ ID NO: 21 - DNA, 27 bp, Homo sapiens
actcgtcgtatttctcagacaagccag

SEQ ID NO: 22 - protein, 9 aa, Homo sapiens
TRRISQTSQ

SEQ ID NO: 23 - DNA, 27 bp, Artificial Sequence
  Description of Artificial Sequence Synthetic oligonucleotide
actcgtcgtattgctcagacaagccag

SEQ ID NO: 24 - protein, 9 aa, Artificial Sequence
  Description of Artificial Sequence Synthetic peptide
TRRIAQTSQ

SEQ ID NO: 25 - DNA, 28 bp, Homo sapiens
aatgatctaggtttctgtggacgctgcc

SEQ ID NO: 26 - protein, 6 aa, Homo sapiens
VSVDAA

SEQ ID NO: 27 - DNA, 28 bp, Artificial Sequence
  Description of Artificial Sequence Synthetic oligonucleotide
aatgatctaggttgctgtggacgctgcc

SEQ ID NO: 28 - protein, 6 aa, Artificial Sequence
  Description of Artificial Sequence Synthetic peptide
VAVDAA